(19) United States
(12) Patent Application Publication
(10) Pub. No.: US 2024/0307290 A1
(43) Pub. Date: Sep. 19, 2024

(54) COSMETIC COMPOSITION COMPRISING A COPOLYMER BASED ON ACETOACETATE FUNCTIONS

(71) Applicant: L'OREAL, Paris (FR)

(72) Inventors: Laurent SABATIE, Aulnay-Sous-Bois (FR); Julien PORTAL, Aulnay-Sous-Bois (FR)

(21) Appl. No.: 18/258,911

(22) PCT Filed: Dec. 16, 2021
(86) PCT No.: PCT/EP2021/086180

§ 371 Date: Jun. 22, 2023

(30) Foreign Application Priority Data
Dec. 23, 2020 (FR) FR2014085
Aug. 5, 2021 (FR) FR2108536

(51) Int. Cl. A61K 8/81; A61Q 1/06; A61Q 5/10

(52) U.S. Cl. CPC A61K 8/8152; A61Q 1/06; A61Q 5/10; A61K 2800/884

(57) ABSTRACT

The present invention concerns a composition containing at least one fatty phase and at least one copolymer obtained by polymerization of 0% to 99% by weight, relative to the total weight of the monomers of at least one monomer (A) chosen from 2-ethylhexyl acrylate, 2-ethylhexyl methacrylate, isobornyl acrylate, isobornyl methacrylate, and mixtures thereof, 1% to 20% by weight of at least one monomer (B) of formula (I), and 0% to 99% by weight of at least one monomer (C) chosen from C₁-C₄ alkyl acrylates, C₁-C₄ alkyl methacrylates, silicone macromonomers and mixtures thereof, relative to the total weight of the monomers,

### TECHNICAL FIELD

The present invention relates more particularly to the cosmetic field of keratin materials, and notably to that of caring for and/or making up the skin and/or lips and/or eyelashes and/or eyebrows, and that of caring for, styling and/or dyeing keratin fibers and preferably the hair. The invention is thus directed toward proposing novel compositions, notably cosmetic compositions, comprising a copolymer containing acetoacetate units, which are most particularly interesting with respect to their technical performance, notably in terms of the persistence of the deposits that they make it possible to obtain, and the sensory sensations that they give the user, and also to corresponding treatment processes, notably cosmetic processes.

### PRIOR ART

Cosmetic products conventionally require the use of one or more film-forming polymers in order to obtain a quality deposit of these products on keratin materials, and in particular to satisfy the expectations detailed below.

Thus, in the field of skin and/or lip makeup, it is most particularly expected that the deposit formed does not transfer on contact with the fingers or clothing.

It must also have good resistance to contact with water, notably rain or during showering or even perspiration, and also to sebum, or even to contact with food fats, notably food oils when this deposit is formed on the lips. Moreover, this deposit must be comfortable or even glossy.

For this purpose, dispersions of polymer particles of nanometric size are used in makeup products such as mascaras, eyeliners, eyeshadows or lipsticks, and more particularly in their organic and notably oily phases, as film-forming agent.

WO 2013/103528 proposes copolymers, not organized in particulate form, in which one of the monomers bears an acetoacetate function, notably for hair applications. WO 93/24098 and WO 95/06454 describe aqueous dispersions containing copolymers, in which one of the monomers bears an acetoacetate function, intended for application to the nails. As regards WO 2014/098052, it proposes an acrylic resin emulsion for styling agents that has been stably dispersed with a polyvinyl alcohol resin. However, the compositions of these documents have unsatisfactory styling hold over time.

Moreover, in the field of haircare, a new range of products known as “Hair Makeup” has recently been developed. These products guarantee temporary hair dyeing that lasts after 1 to 3 shampoo washes. They are thus a particularly attractive alternative for consumers to permanent hair dyeing, provided, of course, that the coloring effect is effectively guaranteed to last after contact with water and a few shampoo washes. This requirement is also notably satisfied by the use of effective film-forming agents. Thus, FR 2 741 530 proposes for this purpose, for the temporary dyeing of keratin fibers, the use of a dispersion of film-forming polymer particles comprising at least one acidic function and at least one pigment dispersed in the continuous phase of said dispersion. The colorings obtained via this dyeing method nevertheless have the drawback of being removed easily on shampoo washing.

It is moreover proposed in FR 2 907 678 to perform colored coating of the hair using a composition comprising a polysiloxane/polyurea block copolymer and a pigment. However, with such a composition, the coating results obtained are not always very homogeneous and the hair strand separation is not always very good.

It is also known practice from patent EP 1 392 222 to use a cosmetic composition for caring for and/or treating keratin materials, comprising a supramolecular polymer including a polymer backbone and at least two groups that are capable of forming at least three hydrogen bonds, and from patent EP 1 435 900 to use a hair composition comprising a supramolecular polymer including a polymer backbone and at least two groups that are capable of forming at least three hydrogen bonds and a surfactant or a hair-conditioning agent. However, with these two composition alternatives, the performance of the styling hold over time or with respect to water remains insufficient.

In general, the polymers described above do not make it possible to obtain deposits, either on keratin fibers or on the skin and lips, which satisfy all the abovementioned requirements, namely very good water resistance, in particular to shampoo washing for the hair, and/or to greasy substances notably for the lips, which are moreover comfortable to wear for the users, which make it possible to adjust the gloss or invisibility qualities and which, in the case of the hair use, provide very satisfactory styling hold.

There is thus still a need for cosmetic compositions intended for application to the skin which make it possible to obtain a deposit that is non-tacky, which transfers little, if at all, and which is glossy, comfortable and long-lasting.

There is also a need for compositions which are resistant to water and to fatty substances, in particular to sebum.

There also remains a need for compositions that provide sheen and invisibility and/or a volumizing effect.

There is also a need for cosmetic dye compositions intended for application to the hair which have good resistance to water and shampoo washing in order to ensure that the color persists over time in a manner comparable to direct dyeing.

There is also a need for cosmetic compositions, notably non-coloring compositions, intended for hair application, which have water-resistant styling properties, notably curl hold.

The present invention is specifically directed toward meeting all or some of these needs.

### SUMMARY OF THE INVENTION

Thus, according to a first of its aspects, the present invention relates to a composition, termed “C1”, notably a cosmetic composition for keratin materials, and in particular for caring for and/or making up the skin, lips, eyelashes and/or eyebrows and/or for caring for, styling and/or coloring keratin fibers, preferably the hair, comprising:
i) at least one fatty phase; and
ii) at least one copolymer, termed “CP”, obtained by polymerization of:
   0% to 99% by weight, in particular from 50% to 99% by weight, relative to the total weight of the monomers, of at least one monomer (A) chosen from 2-ethylhexyl acrylate, 2-ethylhexyl methacrylate, isobornyl acrylate, isobornyl methacrylate, and mixtures thereof,
   1% to 20% by weight, relative to the total weight of the monomers, at least one monomer (B) of formula (I):

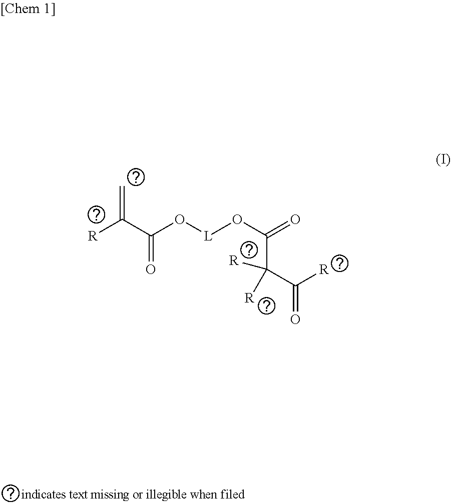

in which:
R^{a} represents a hydrogen atom or a linear or branched (C₁-C₄)alkyl group; preferably, R^{a} represents a methyl group,
R^{b} and R^{c}, which may be identical or different, represent a hydrogen atom or a linear or branched (C₁-C₄)alkyl group; preferably, R^{b} and R^{c} represent a hydrogen atom,
R^{d} represents a linear or branched (C₁-C₄)alkyl group; preferably, R^{d} represents a methyl group, and
L represents a linear or branched (C₁-C₆)alkylene or cycloalkylene group; in particular, L represents a (C₁-C₄)alkylene group; preferably, L represents ethylene;
and preferably monomer (B) is chosen from acetoacetoxyethyl acrylate, acetoacetoxyethyl methacrylate, and mixtures thereof, and
   0% to 99% by weight, in particular 0% to 45% by weight, of at least one monomer (C) chosen from C₁-C₄ alkyl acrylates, C₁-C₄ alkyl methacrylates, silicone macromonomers and mixtures thereof, relative to the total weight of the monomers,
      it being understood that the copolymer termed “CP” is obtained by polymerization of at least one monomer (B) with at least one monomer (A) or at least one monomer (C).

To the inventors' knowledge, obtaining a cosmetic composition from a copolymer comprising at least one monomer (B) with at least one monomer (A) and/or at least one monomer (C) according to the invention, in the mass proportions as defined above, has never been described in the prior art.

According to another aspect, the present invention relates to a composition, termed “C2”, notably a cosmetic composition for keratin materials, and in particular for caring for and/or making up the skin, lips, eyelashes and/or eyebrows and/or for caring for, styling and/or dyeing keratin fibers, preferably the hair, comprising:
i) at least one fatty phase;
ii) at least one copolymer CP as defined previously; and
iii) at least one crosslinking agent termed “R” as defined below.

Preferably, the present invention relates to a composition C2 comprising at least one crosslinking agent, termed “R”, and optionally at least one cosmetic active agent, termed “CAA”, chosen from a) coloring agents chosen from pigments, direct dyes, and mixtures thereof, b) active agents for caring for keratin materials, preferably the skin, c) UV-screening agents, and d) mixtures thereof.

According to yet another aspect, the present invention relates to a composition, termed “C3”, notably a cosmetic composition for keratin materials, and in particular for caring for and/or making up the skin, lips, eyelashes and/or eyebrows and/or for caring for, styling and/or dyeing keratin fibers, preferably the hair, comprising:
i) at least one fatty phase,
ii) at least one copolymer CP as defined previously,
iii) at least one crosslinking agent R as defined below, and
iv) at least one cosmetic active agent, termed “CAA”, notably chosen from a) dyestuffs chosen from pigments, direct dyes, and mixtures thereof, b) active agents for caring for keratin materials, preferably the skin, c) UV-screening agents, and d) mixtures thereof.

Thus, preferably, the present invention also relates to a composition C3 comprising at least one cosmetic active agent, termed “CAA”, chosen from a) coloring agents chosen from pigments, direct dyes, and mixtures thereof, b) active agents for caring for keratin materials, preferably the skin, c) UV-screening agents, and d) mixtures thereof.

According to another of its aspects, the present invention relates to a process, notably a cosmetic process for keratin materials, and in particular for caring for and/or making up the skin, lips, eyelashes and/or eyebrows and/or for caring for, styling and/or dyeing keratin fibers, preferably the hair, comprising a step of applying to said keratin materials a composition C1, C2 or C3, preferably C2 or C3 according to the invention.

Thus, the inventors have found, surprisingly, that a copolymer CP in accordance with the invention makes it possible to obtain cosmetic compositions C1, C2 or C3, notably C2 or C3, leading to deposits for making up the skin, lips, eyelashes and/or eyebrows, which have good resistance to external attacking factors, for instance water, oils, notably food oils, sweat and/or sebum, and thus are endowed with very good staying power over time. Advantageously, the deposits obtained with such compositions are also very comfortable.

The inventors have also observed that a composition C1, C2 or C3 in accordance with the invention, notably a composition C2 or C3, makes it possible to obtain a deposit that is more resistant to external attacking factors, for instance water, oils, notably food oils, sweat and/or sebum, and is thus endowed with very good staying power over time.

The inventors have also observed that cosmetic compositions C1, C2 or C3, notably C2 or C3, in accordance with the invention and more particularly intended for hair application, make it possible to obtain deposits which have very good resistance to shampoo washing and to water.

According to one of its aspects, the present invention relates to a process, notably a cosmetic process, for keratin materials, in particular for caring for and/or making up the skin, lips, eyelashes and/or eyebrows and/or for caring for, styling and/or dyeing keratin fibers and preferably the hair, which consists in applying to said keratin materials a composition C1 according to the invention, optionally at least one crosslinking agent R, in particular according to the invention, and optionally at least one cosmetic active agent CAA, said CAA being chosen from a) coloring agents chosen from pigments, direct dyes, and mixtures thereof, b) active agents for caring for keratin materials, preferably the skin, c) UV-screening agents, and d) mixtures thereof.

According to another of its aspects, the present invention relates to a process for making up the skin, notably the lips, comprising a step of applying to said skin at least one composition C1 or C2 or C3 according to the invention, and at least one dyestuff, notably with said process comprising at least the two successive steps consisting in sequentially applying to said skin i) at least one composition C1 according to the invention, and ii) a crosslinking agent R or a composition CR containing at least one crosslinking agent R notably according to the invention, it being understood that said composition C1 and/or said composition CR contains at least one dyestuff, or said process comprising at least the three successive steps consisting in sequentially applying to the skin i) a composition CAC containing at least one dyestuff, ii) a composition C1 according to the invention, and iii) a crosslinking agent R or a composition CR containing at least one crosslinking agent R, in particular according to the invention, with said composition C1 and/or said composition CR optionally containing at least one dyestuff.

According to another of its aspects, the present invention relates to a process for the care, in particular nontherapeutic and cosmetic care, of the skin, notably of the face or lips, comprising a step of applying to said skin at least one composition C1 or C2 or C3 according to the invention and at least one moisturizing cosmetic active agent; with said process notably comprising at least the two successive steps consisting in applying sequentially to said skin: i) a composition C1 according to the invention, and ii) a crosslinking agent R or a composition CR containing at least one crosslinking agent R in particular according to the invention; it being understood that said composition C1 and/or said composition CR contains at least one moisturizing cosmetic active agent, in particular glycerol, and optionally at least one dyestuff, or said process comprising at least the three successive steps consisting in sequentially applying to the skin i) a composition CAC, ii) a composition C1 according to the invention, and iii) a crosslinking agent R or a composition CR containing at least one crosslinking agent R in particular according to the invention with said composition CAC and/or composition C1 and/or composition CR containing at least one moisturizing cosmetic active agent, in particular glycerol, and optionally a dyestuff.

According to another of its aspects, the present invention relates to a process for styling keratin fibers, notably human keratin fibers, in particular the hair, comprising a step of applying to said keratin fibers a composition C1, C2 or C3 according to the invention.

According to yet another of its aspects, the present invention relates to a process for dyeing keratin fibers, preferably human keratin fibers such as the hair and the eyebrows, comprising a step of applying to said keratin fibers a composition C1, C2 or C3 according to the invention and of at least one coloring agent, with said process notably comprising at least the two successive steps consisting in sequentially applying to said fibers i) at least one composition C1 according to the invention, and ii) a crosslinking agent R or a composition CR containing at least one crosslinking agent R in particular according to the invention, it being understood that said composition C1 and/or said composition CR contains at least one dyestuff, or said process comprising at least the three successive steps consisting in sequentially applying i) a composition CAC containing at least one dyestuff, ii) a composition C1 according to the invention and iii) a crosslinking agent R or a composition CR containing at least one crosslinking agent R in particular according to the invention with said composition C1 and/or said composition CR containing, where appropriate, at least one dyestuff.

According to yet another of its aspects, the present invention is also directed toward a kit, notably a cosmetic kit, for keratin materials, in particular for caring for and/or making up the skin, lips, eyelashes and/or eyebrows, and/or for caring for, styling and/or dyeing keratin fibers and preferably the hair, comprising:
a first compartment containing at least one composition C1 according to the invention, and preferably free of crosslinking agent R;
a second compartment separate from said first compartment and comprising at least one crosslinking agent R, in particular according to the invention; and
where appropriate, a third compartment separate from the first and second compartments and containing at least one cosmetic active agent, termed “CAA”, which may be identical to or different from those optionally contained in the first and/or second compartments;
   it being understood that the first and/or second compartments may also contain one or more cosmetic active agents CAA, which may be identical or different, and in that said CAA is chosen from a) coloring agents chosen from pigments, direct dyes and mixtures thereof, b) active agents for caring for keratin materials, preferably the skin, c) UV-screening agents and d) mixtures thereof.

According to yet another of its aspects, the present invention is also directed toward a kit, notably a cosmetic kit, for keratin materials, in particular for caring for and/or making up the skin, lips, eyelashes and/or eyebrows, and/or for caring for, styling and/or dyeing keratin fibers and preferably the hair, comprising:
a first compartment containing at least one fatty phase, at least one copolymer CP as defined above and preferably free of crosslinking agent R as defined below;
a second compartment separate from said first compartment, comprising at least one crosslinking agent, in particular a crosslinking agent R, which is in particular suitable for use in the present invention; and
where appropriate, a third compartment, separate from the first and second compartments and containing at least one cosmetic active agent, termed “CAA”, which may be identical to or different from those contained in the first and/or second compartment(s);
   it being understood that the first and/or second compartments may also contain one or more identical or different cosmetic active agents CAA, and that said active agent CAA is chosen from a) coloring agents chosen from pigments, direct dyes and mixtures thereof, b) active agents for caring for keratin materials, preferably the skin, c) UV-screening agents and d) mixtures thereof.

For the purpose of the present invention, the term “keratin materials” is notably understood to denote the lips, skin, nails and keratin fibers, in particular the eyelashes, eyebrows and hair, preferably the lips and/or the hair.

A composition according to the invention is generally suitable for application to keratin materials, in particular application to the skin, lips and/or keratin fibers, and thus generally comprises a physiologically acceptable medium, i.e. a medium that is compatible with keratin materials, in particular application to the skin, lips and/or keratin fibers, notably human keratin fibers such as the hair.

It is preferably a cosmetically acceptable medium, i.e. a medium which has a pleasant color, odor and feel and which does not cause any unacceptable discomfort, i.e. stinging or tautness, liable to discourage the user from applying this composition.

For the purposes of the present invention, the terms “dyestuff” and “coloring agent” are equivalent.

### DETAILED DESCRIPTION

As specified above, a composition C1 according to the invention contains i) at least one fatty phase, and ii) at least one copolymer CP as defined previously.

### Copolymer CP

As mentioned above, a composition according to the invention contains at least one copolymer CP obtained by the polymerization of 0% to 99% by weight of at least one monomer (A) chosen from 2-ethylhexyl acrylate, 2-ethylhexyl methacrylate, isobornyl acrylate, isobornyl methacrylate, and mixtures thereof, 1% to 20% by weight of at least one monomer (B) of formula (I) as defined above, and preferably chosen from acetoacetoxyethyl acrylate, acetoacetoxyethyl methacrylate, and mixtures thereof, and 0% to 99% by weight of at least one monomer (C) chosen from C₁-C₄ alkyl acrylates, C₁-C₄ alkyl methacrylates, silicone macromonomers and mixtures thereof, relative to the total weight of the monomers, it being understood that the copolymer termed “CP” is obtained by polymerizing at least one monomer (B) with at least one monomer (A) and/or at least one monomer (C).

Said copolymer CP may be a statistical, alternating (block) or gradient copolymer.

Preferably, the copolymer is a statistical copolymer.

For the purposes of the present invention, the term “statistical copolymer” means a copolymer formed of macromolecules in which the sequential distribution of the monomer units (B), (A) if present, and (C) if present, obeys known statistical laws. In other words, in a statistical copolymer, the various monomers follow each other in any order. Statistical copolymers are also known as random copolymers. For example, the sequence of a random copolymer, formed from monomers A and B, may be as follows A-A-B-A-B-B-B-B-A-B-A.

The copolymer termed “CP” considered according to the invention is in particular free of a monomer unit different from a monomer (B), (A) and (C).

Preferably, the copolymer CP is present in a composition C1, C2 or C3 according to the invention in a content ranging from 1% to 70% by weight, preferably ranging from 2% to 65% by weight, and more preferentially ranging from 3% to 60% by weight, relative to the total weight of the composition.

### Monomer (A)

As mentioned above, the copolymer CP according to the invention may comprise at least one monomer (A) chosen from 2-ethylhexyl acrylate, 2-ethylhexyl methacrylate, isobornyl acrylate, isobornyl methacrylate, and mixtures thereof.

According to a first variant of the invention, the copolymer CP according to the invention does not comprise any monomer (A). In other words, the copolymer CP according to the invention then consists of monomer(s) (B) and monomer(s) (C) as defined above.

According to a second variant of the invention, the copolymer CP comprises from 50% to 99% by weight, and preferably from 55% to 95% by weight, of at least one monomer (A) chosen from 2-ethylhexyl acrylate, 2-ethylhexyl methacrylate, isobornyl acrylate, isobornyl methacrylate, and mixtures thereof, relative to the total weight of the monomers. 2-Ethylhexyl acrylate, also known as 2-ethylhexyl prop-2-enoate, is a compound of empirical formula C₁₁H₂₀O₂, and of structural formula:

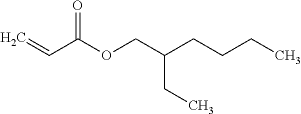

As 2-ethylhexyl acrylate, mention may notably be made of the product sold under the name 2-Ethylhexyl acrylate by the company Sigma-Aldrich.

2-Ethylhexyl methacrylate, also known as 2-ethylhexyl 2-methylprop-2-enoate, is a compound of empirical formula C₂H₂₂O₂ and of structural formula:

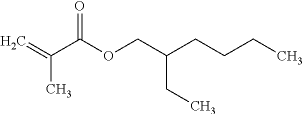

As 2-ethylhexyl methacrylate, mention may notably be made of the product sold under the name 2-Ethylhexyl methacrylate by the company Sigma-Aldrich.

Isobornyl acrylate is a compound of empirical formula C₁₃H₂₀O₂, and of structural formula:

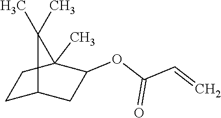

As isobornyl acrylate, mention may notably be made of the product sold under the name Isobornyl acrylate by the company Sigma-Aldrich.

Isobornyl methacrylate is a compound of empirical formula C₁₄H₂₂O₂, and of structural formula:

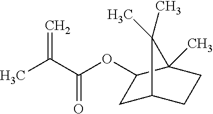 [SMILES: C=C(C)C(=O)OC1CC2CCC1(C)C2(C)C]

As isobornyl methacrylate, mention may notably be made of the product sold under the name Isobornyl methacrylate by the company Sigma-Aldrich.

Preferably, monomer (A) is at least 2-ethylhexyl acrylate and/or isobornyl acrylate.

According to a third variant of the invention, the copolymer CP comprises at least one monomer (A) chosen from 2-ethylhexyl acrylate, 2-ethylhexyl methacrylate, isobornyl acrylate, isobornyl methacrylate, and mixtures thereof, in an amount of less than 50% relative to the total weight of the monomers, preferably between 0.01% and 49.9% relative to the total weight of the monomers.

### Monomer (B)

According to the invention, the copolymer CP comprises from 1% to 20% by weight, and preferably from 5% to 15% by weight, relative to the total weight of the monomers, of at least one monomer (B) of formula (I):

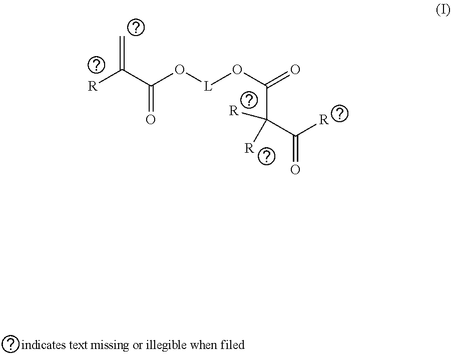

in which:
R^{a} represents a hydrogen atom or a linear or branched (C₁-C₄)alkyl group; preferably, R^{a} represents a methyl group,
R^{b} and R^{c}, which may be identical or different, represent a hydrogen atom or a linear or branched (C₁-C₄)alkyl group; preferably, R^{b} and R^{c} represent a hydrogen atom,
R^{d} represents a linear or branched (C₁-C₄)alkyl group; preferably, R^{d} represents a methyl group, and
L represents a linear or branched (C₁-C₆)alkylene or cycloalkylene group; preferably, L represents a (C₁-C₄)alkylene group, in particular ethylene.

In particular, monomer (B) is chosen from acetoacetoxyethyl acrylate, acetoacetoxyethyl methacrylate, and mixtures thereof, and preferably, monomer (B) is acetoacetoxyethyl methacrylate.

Acetoacetoxyethyl acrylate is a compound of empirical formula C₉H₁₂O₅, and of structural formula:

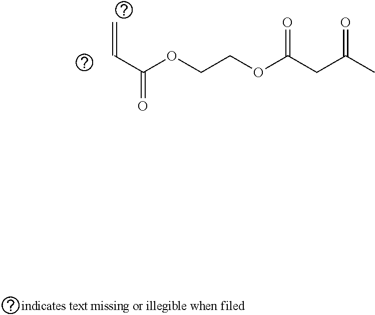

As acetoacetoxyethyl acrylate, mention may notably be made of the product sold under the name Butanoic acid, 3-oxo-2-[(1-oxo-2-propen-1-yl)oxy]ethyl ester by the company Alfa Chemistry.

Acetoacetoxyethyl methacrylate is a compound of empirical formula C₁₀H₁₄O₅, and of structural formula:

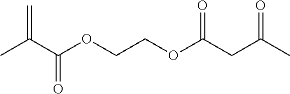

As acetoacetoxyethyl methacrylate, mention may notably be made of the product sold under the name Eastman™ AAEM by the company Eastman.

### Monomer (C)

According to the invention, the copolymer comprises from 0% to 99% by weight of at least one monomer (C) chosen from C₁-C₄ alkyl acrylates, C₁-C₄ alkyl methacrylates, silicone macromonomers and mixtures thereof, relative to the total weight of the monomers.

Thus, according to a variant of the invention, the copolymer CP according to the invention does not comprise any monomer (C). In other words, the copolymer CP according to the invention then consists of monomer(s) (B) and monomer(s) (A) as defined above.

Among the C₁-C₄ alkyl acrylates and C₁-C₄ alkyl methacrylates that are suitable for use in the invention, mention may be made in particular of methyl acrylate, ethyl acrylate, n-propyl acrylate, isopropyl acrylate, n-butyl acrylate, isobutyl acrylate, tert-butyl acrylate, methyl methacrylate, ethyl methacrylate, n-propyl methacrylate, isopropyl methacrylate, n-butyl methacrylate, isobutyl methacrylate, tert-butyl methacrylate and mixtures thereof.

Preferably, monomer (C) is a C₁-C₄ alkyl acrylate, in particular chosen from butyl acrylate, isobutyl acrylate and tert-butyl acrylate.

Butyl acrylate is a compound of empirical formula C₇H₁₃O₂ and of structural formula:

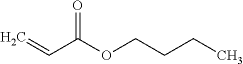

As a butyl acrylate, mention may notably be made of the product sold under the name Butyl acrylate by the company Sigma-Aldrich.

According to a preferred embodiment, monomer (C) is at least one monomer chosen from C₁-C₄ alkyl acrylates and/or C₁-C₄ alkyl methacrylates, preferably chosen from C₁-C₄ alkyl acrylates, and more preferentially monomer (C) is at least butyl acrylate.

Isobutyl acrylate is a compound of empirical formula C₇H₁₂O₂ and of structural formula:

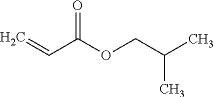

As isobutyl acrylate, mention may notably be made of the product sold under the name Isobutyl acrylate by the company Sigma-Aldrich.

tert-Butyl acrylate is a compound of empirical formula C₇H₁₂O₂ and of structural formula:

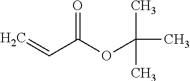

As tert-butyl acrylate, mention may notably be made of the product sold under the name tert-Butyl acrylate by the company Sigma-Aldrich.

According to another preferred embodiment, monomer (C) is at least one silicone macromonomer.

The term “silicone macromonomer” means a silicone macromolecule bearing an end group that enables it to act as a monomer. The silicone macromonomers will provide a single monomer unit to a chain of the finished macromolecule.

As regards the silicone macromonomers, they may in particular be polydimethylsiloxanes bearing a monoacryloyloxy or monomethacryloyloxy end group, and notably those of formula (II):

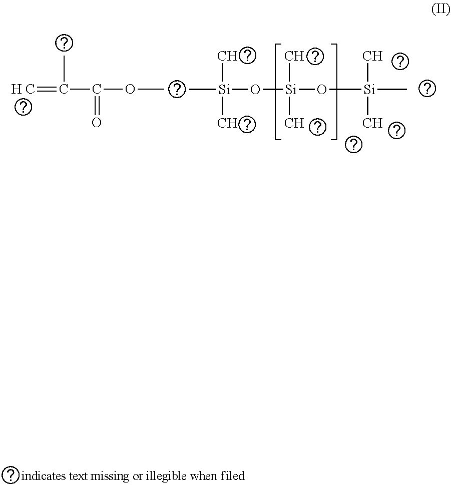

in which:
R₈ denotes a hydrogen atom or a methyl group, preferably methyl;
R₉ denotes a linear or branched, preferably linear, divalent hydrocarbon-based group containing from 1 to 10 carbon atoms and optionally containing one or two —O— ether bonds; preferably ethylene, propylene or butylene;
R₁₀ denotes a linear or branched alkyl group containing from 1 to 10 carbon atoms, notably from 2 to 8 carbon atoms; preferably methyl, ethyl, propyl, butyl or pentyl;
n denotes an integer ranging from 1 to 300, preferentially ranging from 3 to 200.

These may notably be polydimethylsiloxane methacrylates and in particular the products sold under the name MCR-M17 by Gelest Inc. or x-22-2475 and x-22-2426 by Shin-Etsu.

The silicone macromonomers that are most particularly suitable for use in the invention have a weight-average molecular mass (M_{W}) ranging from 200 g·mol⁻¹ to 100 000 g·mol⁻¹, and more preferentially from 400 g·mol⁻¹ to 20 000 g·mol⁻¹.

In particular, monomer (C) is at least one silicone macromonomer, more particularly chosen from silicone macromonomers with a glass transition temperature T_{g} of less than or equal to 25° C., more particularly between −100° C. and 25° C., and preferably between −90° C. and 0° C. According to a preferred embodiment, the monomer (C) is at least one polydimethylsiloxane bearing a mono(meth)acryloyloxy end group.

According to a preferred embodiment of the invention, the copolymer CP comprises from 0% to 45% by weight of at least one monomer (C) chosen from C₁-C₄ alkyl acrylates, C₁-C₄ alkyl methacrylates, silicone macromonomers, and mixtures thereof, relative to the total weight of the monomers, preferably from 0% to 45% by weight of at least one monomer (C) chosen from C₁-C₄ alkyl acrylates, C₁-C₄ alkyl methacrylates, silicone macromonomers, and mixtures thereof, relative to the total weight of the monomers, and at least one monomer (A) as defined previously, more preferentially 50% to 99% of at least one monomer (A) as defined previously.

According to another preferred embodiment of the invention, the copolymer CP comprises from 80% to 99% by weight of at least one monomer (C) chosen from C₁-C₄ alkyl acrylates and C₁-C₄ alkyl methacrylates, and preferably CP comprises from 80% to 99% by weight of at least one monomer (C) chosen from C₁-C₄ alkyl acrylates and, C₁-C₄ alkyl methacrylates relative to the total weight of the monomers, and no monomer (A) as defined previously.

According to another preferred embodiment of the invention, the copolymer CP comprises from 80% to 99% by weight of at least one monomer (C) chosen from silicone macromonomers, and mixtures thereof with C₁-C₄ alkyl acrylates or with C₁-C₄ alkyl methacrylates, relative to the total weight of the monomers, preferably 80% to 99% by weight of a mixture of at least one monomer (C) chosen from silicone macromonomers and at least one monomer (C) chosen from C₁-C₄-alkyl acrylates and/or C₁-C₄-alkyl methacrylates, relative to the total weight of the monomers; more preferentially, the copolymer CP comprises 80% to 99% by weight of a mixture of at least one monomer (C) chosen from silicone macromonomers and at least one monomer (C) chosen from C₁-C₄ alkyl acrylates and/or C₁-C₄ alkyl methacrylates, and no monomer (A) relative to the total weight of the monomers.

According to another particular embodiment of the invention, the copolymer CP comprises more than 30% by weight of at least one monomer (C) chosen from C₁-C₄ alkyl acrylates and C₁-C₄ alkyl methacrylates, and preferably CP comprises from 30.01% to 99% by weight of at least one monomer (C) chosen from C₁-C₄ alkyl acrylates and C₁-C₄ alkyl methacrylates relative to the total weight of the monomers and at least one monomer (A) as defined previously in a non-zero amount and less than 50% relative to the total weight of the monomers. According to this embodiment, the monomers (C) preferably denote C₄ alkyl acrylates, C₄ alkyl methacrylates or mixtures of C₁-C₃ alkyl acrylates and/or C₁-C₃ alkyl methacrylates with C₄ alkyl acrylates or C₄ alkyl methacrylates.

According to another particular embodiment of the invention, the copolymer CP comprises more than 30% by weight of at least one monomer (C) chosen from silicone macromonomers and mixtures thereof with C₁-C₄ alkyl acrylates or with C₁-C₄ alkyl methacrylates, relative to the total weight of the monomers, and preferably CP comprises from 30.01% to 99% by weight of at least one monomer (C) chosen from silicone macromonomers and mixtures thereof with C₁-C₄ alkyl acrylates or with C₁-C₄ alkyl methacrylates, relative to the total weight of the monomers, and at least one monomer (A) as defined previously in a non-zero amount and less than 50% relative to the total weight of the monomers.

According to another particular embodiment of the invention, the copolymer CP comprises more than 45% and less than 49% by weight of at least one monomer (C) chosen from C₁-C₄ alkyl acrylates, C₁-C₄ alkyl methacrylates, silicone macromonomers, and mixtures thereof and preferably CP comprises from 45.01% to 48.99% by weight of at least one monomer (C) chosen from C₁-C₄ alkyl acrylates and C₁-C₄ alkyl methacrylates relative to the total weight of the monomers, and at least one monomer (A) as previously defined in an amount of from 50% to 99% relative to the total weight of the monomers. According to this embodiment, the monomers (C) preferably denote C₄ alkyl acrylates, C₄ alkyl methacrylates or mixtures of C₁-C₃ alkyl acrylates and/or C₁-C₃ alkyl methacrylates with C₄ alkyl acrylates or C₄ alkyl methacrylates.

### Preparation of the Copolymer

The copolymer CP included in a composition C1, C2 or C3 according to the invention may be prepared by radical polymerization of at least one monomer (B) with at least one monomer (A) if present and/or at least one monomer (C) if present and as described previously. The mixture of monomers to be polymerized, generally in an organic medium having a boiling point of greater than or equal to 60° C., may be formed at the very start of the polymerization reaction or may be formed as the polymerization proceeds by adding certain monomers gradually or sequentially. The polymerization of these monomers is performed in the presence of an initiator.

As organic media that are suitable for preparing the copolymer, mention may be made in particular of solvents such as isododecane, ethanol, ethyl acetate, tetrahydrofuran, methyltetrahydrofuran or methyl ethyl ketone and mixtures thereof.

According to one preparation method, the synthetic medium is a mixture of several solvents, notably two solvents of different boiling points. When the synthetic medium contains several solvents of different boiling points, notably two solvents, it is possible, at the end of the synthesis of the copolymer CP, to eliminate the solvent(s) having the lowest boiling points, optionally after adding a medium of higher boiling point which is identical to or different from the solvent of highest boiling point constituting the synthetic medium.

According to this embodiment, the synthetic medium is chosen such that the monomers (B) and (A) and/or (C) of the copolymer and the initiator are soluble therein.

Preferably, the monomers are present in the synthesis solvent, prior to polymerization, in a content ranging from 5% to 45% by weight, relative to the total weight of the synthetic medium.

The monomers to be polymerized can be introduced into the synthetic medium before the start of the polymerization reaction either gradually or sequentially, as the polymerization proceeds.

This polymerization is performed in the presence of an initiator, notably of the peroxide or azo type.

In particular, the initiator may be chosen from tert-butyl peroxy-2-ethylhexanoate, such as Trigonox 21S sold by AkzoNobel, 2,5-dimethyl-2,5-bis(2-ethylhexanoylperoxy)hexane, such as Trigonox 141 sold by AkzoNobel, tert-butyl peroxypivalate, such as Trigonox 25C75 sold by AkzoNobel, azobisisobutyronitrile (AIBN) and 2,2′-azobis(2-amidinopropane) dihydrochloride (V50). Preferably, the initiator is tert-butyl peroxy-2-ethylhexanoate, such as Trigonox 21S sold by AkzoNobel.

Preferably, the polymerization is performed at a temperature ranging from 70° C. to 110° C.

According to a particular form of the invention, the synthesis of the copolymer CP is performed in a mixture of two solvents of different boiling points, in particular in a mixture of isododecane and ethyl acetate. At the end of the reaction, the solvent with the lower boiling point, notably ethyl acetate, is removed, notably by distillation. In this case, at the end of the synthesis, a composition C1 is obtained comprising the copolymer CP in the higher-boiling solvent, notably in isododecane.

According to a preferred embodiment, the copolymers used have weight-average masses of between 5000 g·mol⁻¹ and 1 000 000 g·mol⁻¹, more preferentially between 10 000 g·mol⁻¹ and 500 000 g·mol⁻¹ and even more preferentially between 15 000 g·mol⁻¹ and 350 000 g·mol⁻¹.

As nonlimiting illustrations of copolymers CP used according to the invention, mention may notably be made of the copolymers obtained by copolymerization of
isobornyl acrylate and acetoacetoxyethyl methacrylate, in an isobornyl acrylate/acetoacetoxyethyl methacrylate mass ratio of 90/10,
isobornyl acrylate, 2-ethylhexyl acrylate and acetoacetoxyethyl methacrylate, in an isobornyl acrylate/2-ethylhexyl acrylate/acetoacetoxyethyl methacrylate mass ratio of 60/30/10,
isobornyl acrylate, butyl acrylate and acetoacetoxyethyl methacrylate, in an isobornyl acrylate/butyl acrylate/acetoacetoxyethyl methacrylate mass ratio of 60/30/10, or
isobornyl acrylate, 2-ethylhexyl acrylate, PDMS methacrylate and acetoacetoxyethyl methacrylate, in an isobornyl acrylate/2-ethylhexyl acrylate/PDMS methacrylate/acetoacetoxyethyl methacrylate mass ratio of 60/25/5/10,
isobutyl acrylate, tert-butyl acrylate and acetoacetoxyethyl methacrylate, in an isobutyl acrylate/tert-butyl acrylate/acetoacetoxyethyl methacrylate mass ratio of 25/65/10,
isobutyl acrylate, isobutyl methacrylate and acetoacetoxyethyl methacrylate, in an isobutyl acrylate/isobutyl methacrylate/acetoacetoxyethyl methacrylate mass ratio of 30/60/10,
isobutyl acrylate, 2-ethylhexyl acrylate and acetoacetoxyethyl methacrylate, in an isobutyl acrylate/2-ethylhexyl acrylate/acetoacetoxyethyl methacrylate mass ratio of 88/2/10,
tert-butyl acrylate, ethyl acrylate, isobornyl acrylate and acetoacetoxyethyl methacrylate, in a tert-butyl acrylate/ethyl acrylate/isobornyl acrylate/acetoacetoxyethyl methacrylate mass ratio of 33/20/39/8,
isobornyl acrylate, 2-ethylhexyl acrylate, PDMS methacrylate and acetoacetoxyethyl methacrylate, in an isobornyl acrylate/2-ethylhexyl acrylate/PDMS methacrylate/acetoacetoxyethyl methacrylate mass ratio of 25/20/45/10,
isobutyl acrylate, PDMS methacrylate and acetoacetoxyethyl methacrylate, in an isobutyl acrylate/PDMS methacrylate/acetoacetoxyethyl methacrylate mass ratio of 65/25/10,
isobornyl acrylate, 2-ethylhexyl acrylate, PDMS methacrylate and acetoacetoxyethyl methacrylate, in an isobornyl acrylate/2-ethylhexyl acrylate/PDMS methacrylate/acetoacetoxyethyl methacrylate mass ratio of 40/12/46/2,
isobornyl acrylate, isobutyl acrylate and acetoacetoxyethyl methacrylate, in an isobornyl acrylate/isobutyl acrylate/acetoacetoxyethyl methacrylate mass ratio of 52/46/2.

### Cosmetic Compositions According to the Invention

As stated above, the present invention is also directed toward a composition, notably a cosmetic composition, for keratin materials, in particular for caring for and/or making up the skin, lips, eyelashes and/or eyebrows and/or for caring for, styling and/or dyeing keratin fibers and preferably the hair, comprising a copolymer CP as defined above.

As specified above, this composition according to the invention may notably be in the form of the variants of compositions known as C1, C2 and C3.

Thus, a composition C1 may comprise, in addition to a fatty phase and a copolymer CP in accordance with the invention, at least one cosmetic active agent termed “CAA”, chosen from a) coloring agents chosen from pigments, direct dyes, and mixtures thereof, b) active agents for caring for keratin materials, preferably the skin, c) UV-screening agents, and d) mixtures thereof.

According to a particular form of the invention, a composition C1 consists of a fatty phase and a copolymer CP in accordance with the invention, optionally at least one cosmetic active agent termed “CAA” chosen from a) coloring agents chosen from pigments, direct dyes and mixtures thereof, b) active agents for caring for keratin materials, preferably the skin, c) UV-screening agents, and d) mixtures thereof, and optionally at least one cosmetically acceptable compound different from a cosmetic active agent CAA.

A composition C2 comprises, in addition to a fatty phase and a copolymer CP in accordance with the invention, at least one crosslinking agent termed “R” as defined below, and optionally at least one cosmetic active agent CAA.

According to a particular form of the invention, a composition C2 is free of cosmetic active agent termed “CAA” notably as defined below.

According to another particular form of the invention, a composition C2 consists of a fatty phase and a copolymer CP in accordance with the invention, at least one crosslinking agent, termed “R” as defined below, and optionally a cosmetically acceptable compound other than a cosmetic active agent CAA.

A composition C3 comprises, in addition to a fatty phase and a copolymer CP in accordance with the invention, at least one crosslinking agent, termed “R” as defined below, and at least one cosmetic active agent CAA.

### Crosslinking Agent

For the purposes of the invention, the term “crosslinking agent”, also termed “R”, denotes a compound that is capable of establishing with at least one acetoacetate function of the (co)polymer CP contained in a composition according to the invention:
at least one covalent bond,
at least one donor-acceptor (dative) bond, and/or
at least one coordination bond
   and thus of crosslinking this (co)polymer.

Preferably, the term “crosslinking agent”, also termed “R”, refers to a compound that is capable of establishing at least one covalent bond with an acetoacetate function of the (co)polymer CP contained in a composition according to the invention and thus crosslinking this (co)polymer.

For the purposes of the present invention, it is understood that the terms “crosslinking agent” and “crosslinker” are equivalent.

In a particular embodiment, a composition according to the invention is free of such a crosslinking agent R.

Thus, a composition C1 as previously described comprises a copolymer CP and a fatty phase in accordance with the invention and is free of crosslinking agent R.

In another particular embodiment, compositions according to the invention such as compositions C2 and C3 are obtained by mixing a composition C1 with at least one crosslinking agent R or a composition containing such a crosslinking agent and termed composition “CR”.

As is apparent from the foregoing, a composition CR contains at least one crosslinking agent. In particular, a composition CR does not contain any copolymer CP. A composition CR may, on the other hand, comprise a fatty phase, an aqueous phase or may be in the form of a direct or inverse emulsion. A composition CR may also comprise a cosmetic active agent termed “CAA”, chosen from a) coloring agents chosen from pigments, direct dyes, and mixtures thereof, b) active agents for caring for keratin materials, preferably the skin, c) UV-screening agents, and d) mixtures thereof and notably at least one coloring agent, more particularly at least one pigment.

More particularly, a composition C3 may be obtained either:
by mixing a composition C1 with at least one crosslinking agent R or a composition containing such a crosslinking agent and termed CR, and at least one cosmetic active agent CAA, said CAA being considered via a separate composition termed “CAC”, and/or via the composition C1 and/or CR, or
by mixing a composition C2 with at least one cosmetic active agent CAA or a composition CAC, different from C2, containing said one cosmetic active agent CAA.

As regards a composition CAC, it contains at least one cosmetic active agent chosen from a) coloring agents chosen from pigments, direct dyes, and mixtures thereof, b) active agents for caring for keratin materials, preferably the skin, c) UV-screening agents, and d) mixtures thereof. In particular, a composition CAC does not contain any copolymer CP. A composition CAC also does not contain any crosslinking agent R. A composition CAC may instead comprise a fatty phase, an aqueous phase, or may be in the form of a direct or inverse emulsion.

According to another particular embodiment, a composition C3 may be obtained by mixing a composition C1 according to the invention with a composition CR comprising at least one crosslinking agent R, said composition C1 and/or CR each also possibly containing at least one cosmetic active agent CAA.

When a composition according to the invention contains at least one crosslinking agent, said crosslinking agent and said copolymer CP are preferably present in a mass ratio ranging from 5% to 40%.

More precisely, a crosslinking agent R that is suitable for use in the invention may be chosen from compounds containing amine, thiol, alcohol, acrylate and/or carbonyl functions such as ketone and/or aldehyde functions, or a mixture thereof. A crosslinking agent R may also denote a metal alkoxide or a metal salt or a rare-earth metal derivative.

Thus, according to a particular embodiment, the crosslinking agent R is chosen from (poly)amino, (poly)thiolated and/or (poly)hydroxylated, (poly)carbonyl, (poly)acrylate compounds and mixtures thereof, and preferably chosen from (poly)amino and (poly)thiolated compounds, notably as detailed below.

Thus, according to a particular embodiment, the crosslinking agent R is chosen from (poly)amino, (poly)thiolated and/or (poly)hydroxylated, (poly)carbonyl, (poly)acrylate, and/or metal alkoxide, metal (poly)(hydroxy)(C₁-C₆)alkylcarboxylate, rare-earth metal derivatives and mixtures thereof, and preferably chosen from (poly)amino and (poly)thiolated compounds, notably as detailed below.

The term “(poly)amine, (poly)thiolated and/or (poly)hydroxylated, (poly)carbonyl and (poly)acrylate compounds” denotes that the compounds include at least one amine, thiol and/or hydroxyl, carbonyl such as a ketone or aldehyde function, or acrylate function, respectively.

The metal alkoxide compounds, metal (poly)(hydroxy)(C₁-C₆)alkylcarboxylates and rare-earth metal derivatives are defined below.

### (Poly)amine Crosslinking Agents

According to a preferred embodiment, the crosslinking agent R is chosen from (poly)amine compounds.

The (poly)amine compound may be chosen in particular from polyamine compounds bearing several primary and/or secondary amine groups or from amino alkoxysilanes, and more particularly from amino alkoxysilane compounds, diamine compounds, triamine compounds, and mixtures thereof.

The (poly)amine compound may be a compound comprising from 2 to 20 carbon atoms, notably a non-polymeric compound.

The term “non-polymeric compound” means a compound which is not directly obtained via a monomer polymerization reaction.

(Poly)amine compounds that may be mentioned in particular include N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine, lysine, cystamine, xylenediamine, tris(2-aminoethyl)amine and spermidine.

The amine compound may also be chosen from amino alkoxysilanes, such as those of formula R′₁Si(OR′₂)z(R′₃)x in which:
R′₁ is a linear or branched, saturated or unsaturated, cyclic or acyclic C₁-C₆ hydrocarbon-based chain substituted with a group chosen from the amine groups NH₂ or NHR with R representing a C₁-C₄ alkyl, an aryl or an aryloxy substituted with an amino group or with a C₁-C₄ aminoalkyl group; R′₁ may be interrupted in its chain with a heteroatom (O, S, NH) or a carbonyl group (CO), R′₁ being linked to the silicon atom directly via a carbon atom,
R′₂ and R′₃, which may be identical or different, represent a linear or branched alkyl group comprising from 1 to 6 carbon atoms,
z denotes an integer ranging from 1 to 3, and
x denotes an integer ranging from 0 to 2,
   with z+x=3.

In particular, R′₁ is an acyclic chain. Preferably, R′₁ is a linear or branched, saturated or unsaturated C₁-C₆ hydrocarbon-based chain substituted with an amine NH₂ or NHR group, with R representing a C₁-C₆ alkyl, a C₃-C₆ cycloalkyl or a C₆ aromatic group. More preferentially, R′₁ is a saturated linear C₁-C₆ hydrocarbon-based chain substituted with an amine group NH₂. Even more preferentially, R′₁ is a saturated linear C₂-C₄ hydrocarbon-based chain substituted with an amine group NH₂.

In particular, R′₂ represents an alkyl group comprising from 1 to 4 carbon atoms; preferably, R′₂ represents a linear alkyl group comprising from 1 to 4 carbon atoms and more preferentially R′₂ represents an ethyl group.

In particular, R′₃ represents an alkyl group comprising from 1 to 4 carbon atoms; preferably, R′₃ represents a linear alkyl group comprising from 1 to 4 carbon atoms and more preferentially R′₃ represents methyl or ethyl groups.

Preferably, z is equal to 3.

According to a particular embodiment, the amino alkoxysilane is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane.

Preferably, the amino alkoxysilane is chosen from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane, and more preferentially, the amino alkoxysilane is 3-aminopropyltriethoxysilane (APTES).

According to a preferred embodiment, the (poly)amine compound is chosen from 3-aminopropyltriethoxysilane (APTES), N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine and lysine.

Preferentially, the (poly)amine compound is chosen from ethylenediamine, 1,3-propylenediamine, 1,4-butylenediamine and 3-aminopropyltriethoxysilane (APTES), and more preferentially, the (poly)amine compound is ethylenediamine or 3-aminopropyltriethoxysilane (APTES).

The (poly)amine compound may also be chosen from amino polymers, notably having a weight-average molecular weight ranging from 500 g·mol⁻¹ to 1 000 000 g·mol⁻¹, preferably ranging from 500 g·mol⁻¹ to 500 000 g·mol⁻¹, and preferentially ranging from 500 g·mol⁻¹ to 100 000 g·mol⁻¹.

As amine polymers, mention may be made in particular of poly(C₂-C₅ alkyleneimines), and preferably of polyethylenimines and polypropylenimines, notably poly(ethylenimine), in particular the product sold under the reference 46,852-3 by the company Aldrich Chemical; poly(allylamine), in particular the product sold under reference 47,913-6 by the company Aldrich Chemical; polyvinylamines and copolymers thereof, notably with vinylamides, in particular vinylamine/vinylformamide copolymers such as those sold under the name Lupamin® 9030 by the company BASF; polyamino acids containing NH₂ groups, for instance polylysine, in particular the product sold by the company JNC Corporation (formerly Chisso); amino dextran, in particular the product sold by the company CarboMer Inc; amino polyvinyl alcohol, in particular the product sold by the company CarboMer Inc; copolymers based on acrylamidopropylamine; chitosans, for instance the poly(D-glucosamine) sold under the reference Kionutrime CSG® by the company Kytozyme; polydimethylsiloxanes comprising primary amine groups at the end of the chain or on side chains, for example terminal or lateral aminopropyl groups, for instance those of formula (III) or (IV) or (V):

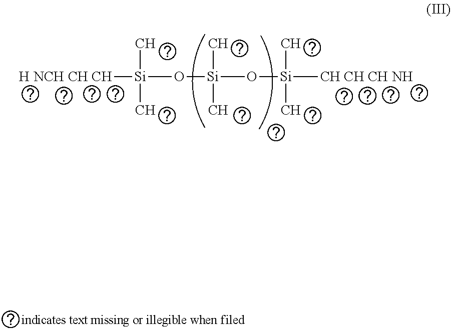

in which the value of n is such that the weight-average molecular weight of the poly dimethylsiloxane ranges from 500 g·mol⁻¹ to 55 000 g·mol⁻¹. As examples of compounds of formula (III), mention may be made of those sold under the names DMS-AT 1, DMS-A12, DMS-A15, DMS-A21, DMS-A31, DMS-A32 and DMS-A35 by the company Gelest.

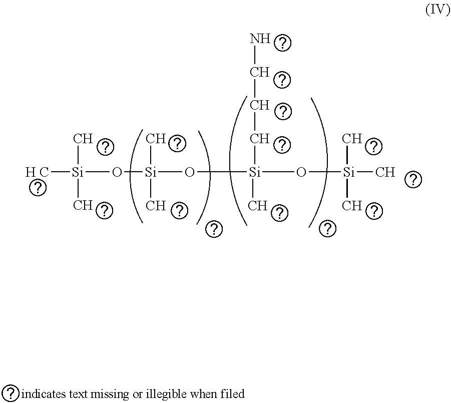

in which the values of n and m are such that the weight-average molecular weight of the polydimethylsiloxane ranges from 1000 g·mol⁻¹ to 55 000 g·mol⁻¹. As examples of poly dimethylsiloxanes of formula (IV), mention may be made of those sold under the names AMS-132, AMS-152, AMS-162, AMS-163, AMS-191 and AMS-1203 by the company Gelest.

H₂NCH₂CH₂CH₂—Si(CH₃)₂—O—[Si(CH₃)₂—O]n—Si(CH₃)₂C₄H₉ (V)

in which the value of n is such that the weight-average molecular weight of the polydimethylsiloxane ranges from 500 to 3000 g·mol⁻¹. As examples of polydimethylsiloxanes (V), mention may be made of the products sold under the names MCR-A11 and MCR-A12 by the company Gelest.

As amine polymers, mention may also be made of the amodimethicones of formula (VI):

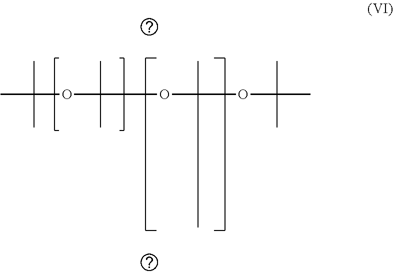

in which R, R′ and R″, which may be identical or different, each represent a hydroxyl or (C₁-C₄)alkyl group; A represents a C3 alkylene group, and m and n are such that the weight-average molecular mass of the compound of formula (VI) ranges from 5000 g·mol⁻¹ to 500 000 g·mol⁻¹.

As amine polymers, mention may also be made of polyether amines, in particular those known under the reference Jeffamine from the company Hunstman; and notably the polyethylene glycol and/or polypropylene glycol α,ω-diamines (with an amine function at the end of the chain), for instance those sold under the names Jeffamine D-230, D-400, D-2000, D-4000, ED-600, ED-9000, ED-2003.

As amine polymers, mention may also be made of α,ω-diamine polytetrahydrofuran (or polytetramethylene glycol), α,ω-diamine polybutadienes and amine-terminated polyamidoamine (PAMAM) dendrimers.

As amine polymers, mention may also be made of poly(meth)acrylates or poly(meth)acrylamides bearing primary or secondary side amine functions, such as poly(3-aminopropyl)methacrylamide, poly(2-aminoethyl)methacrylate.

As amine polymers, use is preferably made of chitosans, polydimethylsiloxanes comprising primary amine groups at the chain end or on side chains.

Preferably, the polyamine compounds are chosen from chitosans, polydimethylsiloxanes comprising primary amine groups at the chain end or on side chains, and APTES.

Thus, according to a preferred embodiment, a composition C2 or C3 according to the invention comprises a crosslinking agent R chosen from (poly)amine compounds, in particular chosen from chitosans, aminoalkoxysilanes, polydimethylsiloxanes comprising primary amine groups at the end of the chain or on side chains, amodimethicones, polyglucosamines, and mixtures thereof.

More preferentially, a composition C2 or C3 according to the invention comprises a crosslinking agent R chosen from chitosans, aminoalkoxysilanes and poly dimethylsiloxanes comprising primary amine groups at the end of the chain or on side chains, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane and polydimethylsiloxanes comprising terminal aminopropyl groups at the end of the chain, and even more preferentially 3-aminopropyltriethoxysilane (APTES).

(Poly)thiolated and/or (poly)hydroxylated Crosslinking Agents

According to a preferred embodiment, the crosslinking agent is chosen from (poly)thiolated and/or (poly)hydroxylated compounds.

The (poly)thiolated and/or (poly)hydroxylated compound may in particular be organic or inorganic, preferably organic.

The (poly)thiolated and/or (poly)hydroxylated compound of the invention is chosen from liposoluble or non-liposoluble compounds.

The term “liposoluble compound” means a compound that is soluble or miscible to at least 1% by weight in isododecane at 25° C.

In a preferred embodiment, the (poly)thiolated and/or (poly)hydroxylated compound is silicon-based, i.e. it includes one or more hydroxyl groups, or one or more thiol groups or one or more hydroxyl groups and one or more thiol groups, and it also includes at least one siloxane chain.

In a particular embodiment, the (poly)thiolated and/or (poly)hydroxylated compound is inorganic. Mention may be made, for example, of polythiol silicones and polythiol silicas.

The (poly)thiolated and/or (poly)hydroxylated compound used in a composition according to the invention may in particular be chosen from non-polymeric (poly)thiolated and/or (poly)hydroxylated compounds.

For the purposes of the present invention, the term “non-polymeric compounds” means compounds which are not directly obtained via a monomer polymerization reaction.

According to one embodiment of the invention, the (poly)thiolated and/or (poly)hydroxylated compound is organic, non-polymeric and of formula (VII) below and also the solvates thereof such as hydrates:

(HO)ₚL(SH)_{q} (VII)

in which:
p and q, which may be identical or different, represent an integer, it being understood that the sum p+q is greater than or equal to 2, preferably, the sum p+q is inclusively between 2 and 10, preferably between 2 and 5;
L denotes a saturated or unsaturated linear or branched, or a saturated or unsaturated (hetero)cyclic, multivalent (at least divalent) group, in particular comprising from 1 to 500 carbon and/or silicon atoms, more particularly between 2 and 40 carbon and/or silicon atoms, even more particularly between 3 and 30 carbon and/or silicon atoms, preferably between 6 and 20 carbon atoms;
L being optionally interrupted and/or terminated with one or more heteroatoms or groups chosen from O, S, N, Si and C(X), and combinations thereof such as —O—, —O—C(X)—, —N(R)—C(X)— or —Si(R_{c})(R_{d})—O— with R representing a hydrogen atom or a (C₁-C₆)alkyl group such as methyl; and/or
L being optionally substituted with one or more halogen atoms, or a group chosen from Rₐ(R_{b})N— and —(X′)ₐ—C(X)—(X″)_{b}—Rₐ;
X, X′ and X″, which may be identical or different, represent an oxygen or sulfur atom, or a group N(R_{b});
a and b being equal to 0 or 1; preferably, the sum a+b is equal to 1;
Rₐ and R_{b}, which may be identical or different, represent a hydrogen atom or a (C1-C₆)alkyl or aryl(C₁-C₄)alkyl group such as benzyl; preferably, Rₐ and R_{b} represent a hydrogen atom;
R_{c} and R_{d}, which may be identical or different, represent a (C₁-C₆)alkyl, aryl(C₁-C₄)alkyl or (C₁-C₆)alkoxy group.

According to one embodiment of the invention, in the above formula (VII), q is equal to 0 and p is an integer greater than or equal to 2; preferably, p is an integer from 2 to 10, preferably from 2 to 5.

According to a particular embodiment of the invention, the (poly)thiolated and/or (poly)hydroxylated compound is chosen from polyhydroxylated compounds, notably polyhydroxylated compounds comprising from 2 to 20 carbon atoms, in particular non-polymeric polyhydroxylated compound(s).

The (poly)thiolated and/or (poly)hydroxylated compound used according to the invention may be chosen from hydroxyalkoxysiloxane or thioalkoxysiloxane compounds, it being understood that these compounds may also comprise one or more primary or secondary amine groups.

A polyhydroxylated compound used according to the invention is an organic compound comprising at least two hydroxyl functions. This compound may comprise other unreactive chemical functions such as ester, amide, ketone or urethane functions. It is possible to use a mixture of different polyhydroxylated compounds.

According to another variant, the polyhydroxylated compound used according to the invention is an inorganic compound comprising at least two hydroxyl functions. This compound may comprise other unreactive chemical functions such as ester, amide, ketone or urethane functions. It is possible to use a mixture of different polyhydroxylated compounds such as a mixture of organic and mineral polyhydroxylated compounds.

According to another embodiment, the thiolated and/or hydroxylated compound is chosen from polyhydroxylated, polythiolated, and polymeric polyhydroxylated and polythiolated compounds.

According to one embodiment of the invention, the polyhydroxylated compound is a non-polymeric organic compound of formula (VIIa) below:

L(OH)ₚ (VIIa)

in which:
p denotes an integer greater than or equal to 2, preferably between 2 and 10, preferably between 2 and 5 inclusive;
L is as defined previously; preferably, L denotes a saturated or unsaturated linear or branched, or saturated or unsaturated (hetero)cyclic, multivalent (at least divalent) radical comprising between 8 and 30 carbon and/or silicon atoms, preferably between 10 and 20 carbon and/or silicon atoms, L also possibly being interrupted with one or more oxygen atoms, and/or comprising one or more functions chosen from amino, ether, thio ether, ester, thio ester, ketone, thio ketone, amide and thio amide functions.

The polyhydroxylated compound is preferably a diol compound.

Preferably, L denotes a C₈-C₁₈ multivalent radical, which is notably linear.

Preferentially, the polyhydroxylated compound is a liposoluble polyol, in particular a C₈-C₁₈ diol, which is notably linear. Advantageously, the C₈-C₁₈ chain is a hydrocarbon-based chain, i.e. formed from carbon and hydrogen.

In particular, the liposoluble polyol is a linear C₈-C₁₆ and notably C₁₀-C₁₄ diol.

As polyol of formula (VIIa), mention may be made of 1,8-octanediol, 1,10-decanediol, 1,12-dodecanediol, 1,14-tetradecanediol, 1,16-hexadecanediol and 1,18-octadecanediol.

Use is preferably made of 1,10-decanediol, 1,12-dodecanediol or 1,14-tetradecanediol. 1,12-Dodecanediol is preferentially used.

According to a particular embodiment of the invention, the thiolated and/or hydroxylated compound is chosen from polythiolated compounds also known as “polymercapto” compounds.

The polythiolated compounds used according to the invention may be water-soluble or liposoluble, and are preferably liposoluble.

According to a particular embodiment of the invention, the thiolated and/or hydroxylated compound is chosen from polythiolated compounds, notably polythiolated compounds comprising from 2 to 20 carbon atoms.

According to a preferred embodiment, the thiolated and/or hydroxylated compound is thiolated, non-polymeric and of formula (VII) defined above, in which p is 0 and q is an integer greater than or equal to 2, preferably p is an integer between 2 and 10, preferably between 2 and 5 inclusive.

In particular, a polythiolated compound that is suitable for use in the invention is a non-polymeric organic compound of formula (VIIb):

L(SH)_{q} (VIIb)

in which L is as defined in formula (VIIa) above and q represents an integer greater than or equal to 2, preferably between 2 and 10, preferably between 2 and 5 inclusive.

The polythiolated compounds that are suitable for use in the invention are preferably dithiol compounds.

Preferably, L denotes a C₈-C₁₈ multivalent radical, which is notably linear. Preferentially, the liposoluble polythiol is a notably linear C₈-C₁₈ dithiol. Advantageously, the C₈-C₁₈ chain is a hydrocarbon-based chain, i.e. formed from carbon and hydrogen. In particular, the liposoluble polythiol is a linear C₈-C₁₆ and notably C₁₀-C₁₄ dithiol.

As polythiol of formula (VIIb), mention may be made of 1,8-octanedithiol, 1,10-decanedithiol, 1,12-dodecanedithiol, 1,14-tetradecanedithiol, 1,16-hexadecanedithiol and 1,18-octadecanedithiol.

Use is preferably made of 1,10-decanedithiol, 1,12-dodecanedithiol or 1,14-tetradecanedithiol. 1,12-Dodecanedithiol is preferentially used.

According to another embodiment of the invention, the thiolated and/or hydroxylated compound is thiolated and hydroxylated, non-polymeric and of formula (VII) as defined above, in which q and p are integers greater than or equal to 1; preferably, the sum p+q is an integer of from 2 to 10, preferably from 2 to 5, and preferably q>p.

According to yet another particular embodiment of the invention, the thiolated and/or hydroxylated compound is chosen from (poly)hydroxylated and (poly)thiolated compounds, notably (poly)hydroxylated and (poly)thiolated compounds comprising from 2 to 20 carbon atoms, and notably non-polymeric (poly)hydroxylated and (poly)thiolated compounds.

According to another particular embodiment of the invention, the thiolated and/or hydroxylated compound is chosen from hydroxylated and/or thiolated alkoxysiloxanes, such as those of formula (VIII) below:

R′₁—Si(OR′₂)_{z}(R′₃)ₓ (VIII)

in which:
R′₁ is a linear or branched, saturated or unsaturated, cyclic or acyclic C₁-C₁₂ hydrocarbon-based chain substituted with one or more groups chosen from the following groups:
hydroxyl or thiol, preferably thiol,
aryl, aryloxy, arylthio or arylamino, the aryl group being substituted with one or more hydroxyl, thiol, hydroxy(C₁-C₆)alkyl or thio(C₁-C₆)alkyl groups, preferably thio(C₁-C₆)alkyl, and
R′₁ is optionally interrupted in its hydrocarbon-based chain with one or more heteroatoms such as O, S, N, a carbonyl group (CO), or a combination thereof such as ester —C(O)—O—, or amide —C(O)—N(H)—, R′₁ being bonded to the silicon atom directly via a carbon atom,
R′₂ and R′₃, which may be identical or different, represent a linear or branched alkyl group comprising from 1 to 6 carbon atoms, preferably from 1 to 4 carbon atom(s) such as methyl,
z denotes an integer ranging from 1 to 3, and
x denotes an integer ranging from 0 to 2, with z+x=3.

Preferably, R′₂ represents an alkyl group comprising from 1 to 4 carbon atoms.

Preferably, R′₂ represents a linear alkyl group comprising from 1 to 4 carbon atoms.

Preferably, R′₂ represents an ethyl group.

Preferably, R′₃ represents an alkyl group comprising from 1 to 4 carbon atoms.

Preferably, R′₃ represents a linear alkyl group comprising from 1 to 4 carbon atoms.

Preferably, R′₃ represents a methyl or ethyl group.

Preferably, R′₁ is an acyclic chain.

Preferably, R′₁ is a linear or branched, saturated or unsaturated C₁-C₆ hydrocarbon-based chain, substituted with one or more hydroxyl or thiol groups, preferably thiol.

Preferentially, R′₁ is a saturated linear C₁-C₆ hydrocarbon-based chain substituted with a hydroxyl or thiol group, preferably thiol.

More preferentially, R′₁ is a saturated linear C₂-C₄ hydrocarbon-based chain substituted with a hydroxyl or thiol group, preferably thiol.

Preferably, R′₁ is a saturated linear C₁-C₆ hydrocarbon-based chain substituted with a hydroxyl or thiol group, preferably thiol, R′₂ represents an alkyl group comprising from 1 to 4 carbon atoms,
R′₃ represents an alkyl group comprising from 1 to 4 carbon atoms.

Preferably, z is equal to 3.

According to an even more particular embodiment of the invention, the alkoxysiloxanes (VIII) are chosen from those of formula (VIII′):

(R¹O)(R²)(R³)Si—CH₂—[N(R⁴)-L¹]ₚ-X—H (VIII′)

in which:
p is 0 or 1;
X represents an oxygen or sulfur atom, preferably a sulfur atom;
R¹ represents a (C₁-C₆)alkyl radical;
R² and R³, which may be identical or different, preferably identical, are chosen from:
a (C₁-C₆)alkoxy group, in particular C₁-C₄;
a (C₁-C₆)alkyl group;
R⁴ represents a hydrogen atom, or a (C₁-C₆)alkyl group such as methyl;
L¹ represents a divalent, saturated, linear or branched C₁-C₂₀ hydrocarbon-based radical.

According to an even more particular embodiment of the invention, the alkoxysiloxanes (VIII) are chosen from those of formula (VIII″) below:

(R′¹O)(R′²)(R′³)Si—CH(R⁴)—CH(R⁵)-(L²)_{q}-X—H (VIII″)

in which:
q is 0 or 1;
X represents an oxygen or sulfur atom, preferably a sulfur atom;
R′¹ denotes a (C₁-C₆)alkyl radical;
R′² and R′³, which may be identical or different, preferably identical, are chosen from:
a (C₁-C₆)alkoxy group, in particular C₁-C₄;
a (C₁-C₆)alkyl radical;
R⁵ represents a hydrogen atom or a C₁-C₄ alkyl group optionally substituted with an amino, thiol or hydroxyl group;
R⁴ represents a hydrogen atom or a C₁-C₄ alkyl group, in particular methyl;
L² represents a divalent, linear or branched, saturated C₁-C₂₀ hydrocarbon-based group, optionally interrupted with a heteroatom such as —NH—, optionally substituted with one or more hydroxyl, thiol or amino groups.

Preferably, the alkoxysilane of formula (VIII) is chosen from 4-(trimethoxysilyl)-1-butanol, 3-(trimethoxysilyl)-1-propanol, 3-(triethoxysilyl)-1-propanol, 11-(trimethoxysilyl)-1-undecanethiol, 4-(trimethoxysilyl)-2-butanethiol, 2-(triethoxysilyl)ethanethiol, 3-(triethoxysilyl)-1-propanethiol, 2-(trimethoxysilyl)ethanethiol, 3-(trimethoxysilyl)-1-propanethiol and 3-(dimethoxymethylsilyl)-1-propanethiol.

More preferentially, the alkoxysilane of formula (VIII) is chosen from 2-(triethoxysilyl)ethanethiol (18236-15-2) and 3-(triethoxysilyl)-1-propanethiol (14814-09-6). According to another embodiment, the thiol compound is a particle bearing at least two thiol groups. It is, for example, a silica functionalized with radicals, notably of the alkyl type, substituted with thiol functions.

According to this embodiment, the thiol compound may refer to the particles sold under the name SiliaMetS® Thiol by the company Silicycle or under the name SP-THIO-SILICA by the company Suprasciences.

The thiol compound may be chosen from the polythiol silicas prepared according to the procedure described in J. Mater. Chem., 2007, 17, 3726-3732; J. Am. Chem. Soc., 2005, 127 (23), pages 8492-8498; 10.1109/ICBBE.2010.5517542; Chemical Engineering Journal, 2011, 171 (3), 1004-1011; Minerals Engineering, 2012, 35, 20-26; Colloids and Surfaces A: Physicochemical and Engineering Aspects, 380 (1-3), 229-233, Advanced Science, Engineering and Medicine, Volume 5, Number 9, September 2013, pages 984-987(4).

The particle bearing at least two thiol groups may also be obtained by reacting a silica particle such as the Sunsphere® particles sold by Asahi Glass with mercaptoalkylalkoxysilanes such as 3-mercaptopropyltriethoxysilane.

Preferably, the thiol compound is a particle bearing at least two thiol groups obtained by reacting a Sunsphere® silica particle sold by Asahi Glass, preferably a 5 μm Sunsphere particle, with 3-mercaptopropyltriethoxysilane according to the publication, Journal of Chromatography A, Volume 1217, Issue 47, 19 Nov. 2010, pages 7448-7454.

According to a second embodiment, the polythiolated particles may be present in the form of latex bearing thiol groups such as those described in Colloids and Surfaces A: Physiochemical and Engineering Aspects 153, 1999, 421-427.

According to another embodiment, the thiol compound is a particle coated with compound(s) bearing at least two thiol functions.

The (poly)thiolated and/or (poly)hydroxylated compound used in a composition according to the invention may in particular be chosen from polymeric (poly)thiolated and/or (poly)hydroxylated compounds.

The polymeric (poly)thiolated and/or (poly)hydroxylated compounds may be homopolymers, copolymers, star, comb, brush and dendritic compounds bearing hydroxyl and/or thiol units. The polymers may be of natural origin such as polysaccharides or polypeptides, or of synthetic origin such as acrylic polymers, polyesters or polyglycols. The hydroxyl and thiol units may be present as terminal or side groups.

Examples that may be mentioned include the polymers described in the following scientific articles: Polymers containing groups of biological activity, C. G. Overberger et al., Polytechnic Institute of Brooklyn, http://pac.iupac.org/publications/pac/pdf/1962/pdf/0402x0521.pdf; EP 1 247 515 A2; U.S. Pat. No. 3,676,440; and EP 1 572 778.

The thiolated and/or hydroxylated polymers that may be suitable for use in the invention are preferably organic or silicon-based, more preferentially of formula (IX):

(HO)ₚPOLY(SH)_{q} (IX)

in which:
p and q, which may be identical or different, represent an integer, it being understood that the sum p+q is greater than or equal to 3;
POLY denotes a polymeric radical which is preferably carbon-based or silicon-based;
POLY being optionally interrupted with one or more heteroatoms or groups chosen from O, S, N, Si and C(X), and combinations thereof such as —O—, —O—C(X)—, —N(R)—C(X)— or —Si(R_{c})(R_{d})—O— with R representing a hydrogen atom or a (C₁-C₆)alkyl group such as methyl; and/or
POLY being optionally substituted with one or more halogen atoms, or a group chosen from Rₐ(R_{b})N— and —(X′)ₐ—C(X)—(X″)_{b}—Rₐ;
X, X′ and X″, which may be identical or different, represent an oxygen or sulfur atom, or a group N(R_{b});
a and b being equal to 0 or 1; preferably, the sum a+b is equal to 1;
Rₐ and R_{b}, which may be identical or different, represent a hydrogen atom or a (C₁-C₁₀)alkyl or aryl(C₁-C₄)alkyl group such as benzyl; preferably, Rₐ and R_{b} represent a hydrogen atom; and
R_{c} and R_{d}, which may be identical or different, represent a (C₁-C₁₀)alkyl, aryl(C₁-C₄)alkyl or (C₁-C₁₀)alkoxy group.

According to a particular embodiment, the thiolated and/or hydroxylated compound is chosen from (poly)hydroxylated polymers, also known as “polyol”.

According to another embodiment of the invention, the (poly)hydroxylated polymer(s) are chosen from those of formula (IX′):

POLY(OH)ₚ (IX′)

in which:
p denotes an integer greater than or equal to 2, and
POLY denotes a carbon-based or silicon-based polymeric radical, POLY also possibly containing one or more heteroatoms such as O, N or S, and/or one or more functions chosen from amino, (thio)ester, (thio)ketone, (thio)amide, (thio)urea and (thio)carbamate functions, and/or possibly being substituted with one or more linear or branched (C₁-C₁₀)alkyl or linear or branched (C₁-C₁₀)alkoxy groups, it being understood that when POLY is substituted, the hydroxyl groups may be borne by the substituent(s).

The weight-average molecular weight of the polyol polymer compounds, such as those of formula (IX′), is generally between 500 and 400 000 g·mol⁻¹, preferably between 500 and 150 000 g·mol⁻¹.

Preferably, the polyhydroxylated polymers (IX′) may be (di)ol polymers, notably polyolefin (poly)ols, polydi(C₁-C₆)alkylsiloxane (poly)ols or polyester (poly)ols; more preferentially, the (poly)ols are diols.

The polyolefin (poly)ols may be polydienes bearing hydroxyl end groups, for instance those described in FR-A-2 782 723. They may be chosen from (poly)ols derived from homopolymers and copolymers of polybutadiene, of polyisoprene and of poly(1,3-pentadiene). They preferably have a number-average molecular mass (Mn) of less than 7000 g·mol⁻¹, preferably between 1000 and 5000 g·mol⁻¹. Mention will be made in particular of the hydroxylated polybutadienes sold by the company Cray Valley under the brand names Poly BD R45HTLO, Poly BD R45V and Poly BD R-20 LM, which will preferably be used hydrogenated; and also (poly)hydroxylated hydrogenated (1,2-polybutadienes), such as GI3000 of Mn=3100, GI2000 (Mn=2100) and GI1000 (Mn=1500) sold by the company Nisso.

More particularly, the compounds of formula (IX′) are chosen from the polyolefin (poly)ols of formula (X) below:

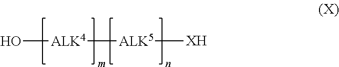

in which:
ALK4 and ALK5, which may be identical or different, preferably different, represent a linear or branched (C₁-C₆)alkylene group, optionally substituted with one or more hydroxyl, thiol or amino groups; preferably, ALK4 represents a linear (C₁-C₆)alkylene group such as n-butylene, and ALK5 represents a branched (C₃-C₆)alkylene group such as i-butylene;
X represents an oxygen or sulfur atom or a group N(Ra) with Ra representing a hydrogen atom or a (C₁-C₄)alkyl group; preferably, X represents a hydroxyl or thiol group, more preferentially hydroxyl; and
n and m, which may be identical or different, represent an integer, with n+m representing an integer greater than or equal to 1.

The (poly)ols of formula (X) may in particular be chosen from polyolefins bearing hydroxyl end groups.

Among the polyolefins bearing hydroxyl end groups, mention may be made preferentially of polyolefin homopolymers or copolymers bearing α,ω-hydroxy end groups, such as polyisobutylenes bearing α,ω-hydroxy end groups and the copolymers of formula (X′):

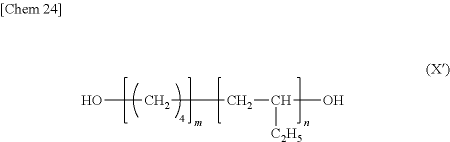

notably those sold by Mitsubishi under the brand name Polytail. Hydrogenated polybutadiene diols are preferably used.

The (poly)ols of formula (X) may in particular be chosen from polydialkylsiloxane (di)ols, particularly from those of formula (XI) below:

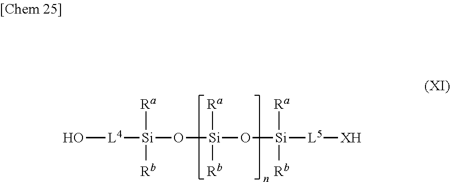

in which:
R^{a} and R^{b}, which may be identical or different, preferably identical, represent a group from among: (C₁-C₆)alkyl optionally substituted with one or more hydroxyl, amino or thiol groups; (C₁-C₆)alkoxy such as methoxy; aryl such as phenyl; aryloxy such as phenoxy; aryl(C₁-C₄)alkyl such as benzyl; or aryl(C₁-C₄)alkoxy such as benzoxy; preferably (C₁-C₄)alkyl such as methyl;
n represents an integer greater than or equal to 1 and more particularly the value of n is such that the weight-average molecular weight of the silicone is between 500 and 55 000 g·mol⁻¹; in particular, n is an integer from 1 to 100, preferably from 5 to 50 and preferentially from 10 to 30; and
L⁴ and L⁵, which may be identical or different, represent a covalent bond or a saturated or unsaturated, linear or branched, optionally cyclic hydrocarbon-based chain comprising from 1 to 100 carbon atoms, optionally interrupted with one or more heteroatoms such as oxygen, sulfur or nitrogen, in particular oxygen, more preferentially a (C₁-C₆)alkylene, (C₁-C₆)alkylenoxy, oxy(C₁-C₆)alkylene, (C₁-C₆)alkylenoxy(C₁-C₆)alkylene, (C₁-C₆)alkylenoxy(C₁-C₆)alkylenoxy or oxy(C₁-C₆)alkylenoxy(C₁-C₆)alkylene group;
X represents an oxygen or sulfur atom, preferably oxygen.

Preferentially, the polydialkylsiloxane (di)ols of formula (XI) are chosen from polydimethylsiloxanes, notably the polydiols of formula (XI′) below:

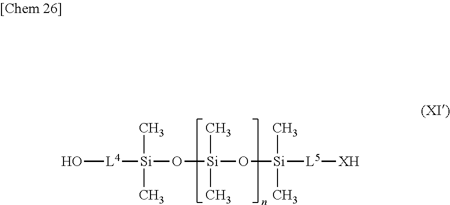

in which:
L⁴ and L⁵ are as defined previously, and preferably represent a divalent group chosen from —R₂—, —O—R₂—, —R₂—O— and —R₂—O—R′₂—, preferably —R₂—O—R′₂—, with R₂ and R′₂, which may be identical or different, representing a linear or branched (C₂-C₆)alkylene group, such as ethylene or propylene; and
n represents an integer between 1 and 100 inclusive, preferably between 5 and 50 and preferentially between 10 and 30.

Polydimethylsiloxanes diols that may be used include those sold under the names KF-6000, KF-6001, KF-6002 and KF-6003 by the company Shin-Etsu Chemicals.

Use is preferably made of the polydimethylsiloxane diol of formula (XI″) below:

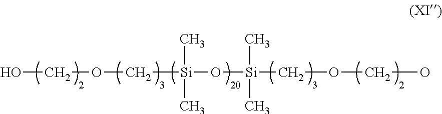

Use may also be made of dimethiconols, which are polydimethylsiloxanes bearing OH terminal functions. Mention may be made, for example, of the product sold under the name Xiameter PMX-1502 Fluid by the company Dow Corning.

According to a particular form of the invention, the (poly)ols of formula (X) are chosen from the polyhydroxylated compounds of formula (XII) below:

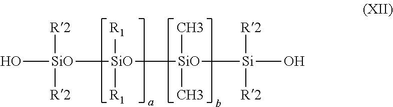

in which:
R₁, which may be identical or different, independently represents a hydroxyl group; an alkyl group containing from 1 to 10 carbon atoms, preferably 1 to 4 carbon atoms, notably 1 to 2 carbon atoms such as a methyl; an alkoxy group containing from 1 to 2 carbon atoms; or a group —(CH₂)ₛ—Si(R₄)₃ in which s denotes an integer ranging from 1 to 4 such as 2 and R⁴ independently denotes an alkoxy radical containing from 1 to 2 carbon atoms;
R′₂ and R″₂ independently represent an alkyl group containing from 1 to 10 carbon atoms, preferably from 1 to 4 carbon atoms, notably 1 to 2 carbon atoms such as a methyl;
a denotes an integer ranging from 0 to 10, b denotes an integer ranging from 0 to 500 with a+b≥4.

Among the compounds of formula (XII), mention may be made of polydimethylsiloxanes (PDMS) bearing hydroxyl terminal functions, such as the compounds sold by the company Shin-Etsu under the name KF-9701 or X-21-5841, or those sold by the company Sigma-Aldrich under the reference 481939 (Mn˜ 550 g·mol⁻¹, ˜25 cSt), 481955 (˜65 cSt) or 481963 (˜750 cSt). Mention may also be made of the compounds sold by the company Gelest under the name DMS-S12 (16-32 cSt), DMS-S15 (45-85 cSt), DMS-S21 (90-120 cSt), DMS-S27 (700-800 cSt) or DMS-S31 (˜1000 cSt).

According to a preferred embodiment, the silicone(s) of formula (XII) used in the context of the invention are chosen from the compounds of formula (XII) in which:
R₁ independently represents an alkyl group containing from 1 to 10 carbon atoms, preferably from 1 to 4 carbon atoms and more particularly from 1 to 2 carbon atoms, such as a methyl;
R′₂ and R″₂ independently represent an alkyl group containing from 1 to 10 carbon atoms, preferably an alkyl group containing from 1 to 4 carbon atoms and more particularly from 1 to 2 carbon atoms such as methyl;
b denotes an integer ranging from 0 to 10, a denotes an integer ranging from 0 to 5 with a+b≥4.

According to another embodiment of the invention, the polythiol compound is a thiolated polymer compound.

The methods for preparing the thiolated polymers used according to the invention are known to those skilled in the art; several methods are reported hereinbelow in a nonlimiting manner.

The thiolated polymers used according to the invention may be obtained by polymerization or polycondensation of monomer units bearing thiol or protected thiol functions, optionally as a copolymerization or co-polycondensation of monomer units free of thiol or protected thiol functions.

Alternatively, the thiolated polymers used according to the invention may be obtained by addition of hydrogen sulfide, of salts thereof such as sodium hydrogen sulfide or potassium sulfide or alternatively a group that is capable of forming a carbon-sulfur bond such as thiourea derivatives or thiosulfate, on a polymer bearing at least one double bond.

The thiolated polymers used according to the invention may also be obtained by nucleophilic substitution of a leaving group present on a polymer chain (for example a halogen such as chlorine or bromine, or a sulfonic ester such as mesylate or tosylate) with a compound including at least one sulfur atom such as those mentioned previously.

The thiolated polymers used according to the invention may also be obtained by reaction of polymers including nucleophilic groups such as amines on electrophilic compounds including a sulfur atom, such as 2-oxo-4-thiazolidinecarboxylic acid, also known as procysteine:
N-acetyl homocysteine thiolactone:

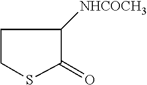

γ-thiobutyrolactone:

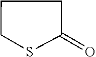

iminothiolane:

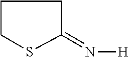

According to one embodiment of the invention, the thiolated polymers used according to the invention are polymers which are soluble in cosmetic media, particularly in aqueous or aqueous-alcoholic media. They are more preferentially obtained from amino polymers and their ammonium salts or from polyhydroxylated polymers.

According to another embodiment of the invention, the thiolated polymers used according to the invention are polymers that are soluble in lipophilic media.

According to one embodiment of the invention, the polythiol compound is a polymeric compound of formula (XIII):

POLY(SH)_{q} (XIII)

in which:
q denotes an integer greater than or equal to 2, and
POLY denotes a carbon-based and/or silicon-based, preferably silicon-based, polymeric radical, POLY also possibly containing one or more heteroatoms such as O, N or S, and/or one or more functions chosen from (thio)ester, (thio)ketone, (thio)amide, (thio)urea and (thio)carbamate functions, and/or possibly being substituted with one or more linear or branched (C₁-C₁₀)alkyl or linear or branched (C₁-C₁₀)alkoxy groups, it being understood that when POLY is substituted, the thiol functions may be borne by the substituent(s).

The weight-average molecular weight of the polythiol polymer compounds, such as those of formula (XIII), is generally between 500 and 400 000 g·mol⁻¹, preferably between 500 and 150 000 g·mol⁻¹.

According to a particular embodiment of the invention, the polythiol compounds are chosen from the polyorganosiloxanes of formula (XIII′):

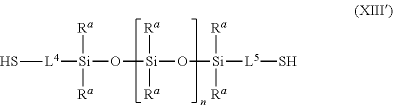

in which:
R^{a} and R^{b}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl,
n represents an integer greater than or equal to 1 and more particularly the value of n is such that the weight-average molecular weight of the silicone ranges from 500 to 55 000 g·mol⁻¹; in particular, n is an integer ranging from 1 to 100, preferably ranging from 5 to 50 and preferentially ranging from 10 to 30, and
L⁴ and L⁵ are as defined previously in formula (XII), in particular represent a covalent bond or a (C₁-C₆)alkylene, (C₁-C₆)alkylene-oxy, oxy-(C₁-C₆)alkylene, (C₁-C₆)alkylene-oxy(C₁-C₆)alkylene, (C₁-C₆)alkylene-oxy(C₁-C₆)alkylenoxy or oxy(C₁-C₆)alkylene-oxy(C1-C₆)alkylene group, preferably a (C₁-C₆)alkylene, (C₁-C₆)alkylene-oxy, oxy-(C₁-C₆)alkylene or (C₁-C₆)alkylene-oxy(C₁-C₆)alkylene group.

Preferentially, the polydimethylsiloxane thiols are chosen from those of formula (XIII″):

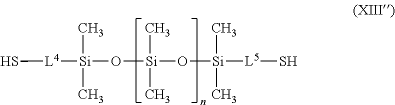

in which:
L⁴ and L⁵ represent a linear or branched, optionally cyclic, saturated or unsaturated hydrocarbon-based chain comprising from 1 to 100 carbon atoms, optionally interrupted with one or more heteroatoms such as oxygen, sulfur or nitrogen, in particular oxygen; preferably L⁴ and L⁵ represent a (C₁-C₆)alkylene, (C₁-C₆)alkylenoxy, oxy(C₁-C₆)alkylene or (C₁-C₆)alkylenoxy(C₁-C₆)alkylene group, more preferentially a divalent group chosen from —R₂—, —O—R₂—, —R₂—O— and —R₂—O—R₂—, preferably —R₂—O—R₂—, with R₂ representing a linear or branched, preferably linear, (C₂-C₆)alkylene group, such as ethylene or n-propylene;
n represents an integer from 1 to 100, preferably from 5 to 50, and preferentially from 10 to 30.

As thiolated poly(C₁-C₄)alkylsiloxanes, mention may be made of mercaptosiloxanes or thiolated siloxanes in which the thiol functions are at the chain ends, sold by the company Shin-Etsu under the reference X-22-167B, and mercaptosiloxane in which the mercapto functions are pendent, sold by the company Shin-Etsu under the reference KF-2001, or polydimethylsiloxanes in which the thiol functions are at the chain ends, via thio-n-propyl, 80-120 groups, sold by the company Gelest under the name DMS-SM 21, of formula (XIII′″)

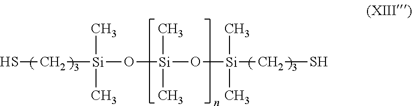

Preferentially, the polythiolated compounds are chosen from those of formula (XIV):

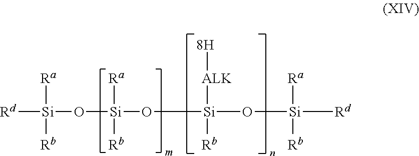

in which:
R^{a}, R^{b} and R^{d}, which may be identical or different, preferably identical, represent a group from among: (C₁-C₆)alkyl optionally substituted with a hydroxyl or amino group, preferably (C₁-C₄)alkyl such as methyl; (C₁-C₄)alkoxy such as methoxy; aryl such as phenyl; aryloxy such as phenoxy; aryl(C₁-C₄)alkyl such as benzyl; or aryl(C₁-C₄)alkoxy such as benzoxy; preferably (C₁-C₄)alkyl such as methyl;
R^{d} may also represent a (C₁-C₆)alkyl group substituted with a (C₁-C₄)alkylamino or amino or thiol group, preferably (C₁-C₄)alkyl such as methyl;
ALK represents a linear or branched, optionally cyclic, saturated or unsaturated hydrocarbon-based chain comprising from 1 to 100 carbon atoms, optionally interrupted with one or more heteroatoms such as oxygen, sulfur or nitrogen (in particular O), a (thio)carbonyl group C(X) with X representing O or S, or combinations thereof such as —O—, —O—C(O)— or —C(O)—O—; preferably, ALK represents a (C₁-C₆)alkylene and more preferentially (C₁-C₄)alkylene group such as propylene;
n and m, which may be identical or different, representing an integer greater than 2 and more particularly the values of m and n are such that the weight-average molecular weight of the silicone is between 1000 and 55 000 g·mol⁻¹.

Preferentially, the polydi(C₁-C₄)alkylsiloxanes of formula (XIV) have the formula (XIV′) below:

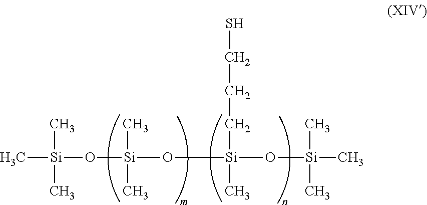

in which the values of n and m are such that the weight-average molecular weight of the silicone is between 1000 and 55 000 g·mol⁻¹.

As examples of silicones of formula (XIV′), mention may be made of those sold by the company Genesee Polymers under the names GP-367, GP-71-SS, GP-800 and GP-710.

Preferably, the silicone of formula (XIV′) is the compound GP-367 sold by the company Genesee Polymers.

The polythiol silicones are notably polydimethylsiloxanes containing at least two thiol groups, for instance the products SMS-022, SMS-042 and SMS-992 sold by the company Gelest in https://www.gpcsilicones.com/products/silicone-fluids/mercapto-functional, https://www.shinetsusilicone-global.com/products/type/oil/detail/search/deg07.shtml, and 1053_Reactive Silicones_Silanes/Silicones—Gelest.

According to another embodiment, the thiolated compound is a particle bearing at least two thiol groups, such as a silica functionalized with radicals, for example of the alkyl type, substituted with thiol functions, or a particle covered with compound(s) bearing at least two thiol functions (coating), said particle possibly being a sphere, a fiber, a rod or an amorphous structure.

According to a particular embodiment of the invention, the hydroxylated and/or thiolated compound is chosen from polymeric compounds such as hyperbranched polymers and dendrimers.

“Hyperbranched polymers” are molecular constructions having a branched structure, generally around a core. Their structure is generally free of symmetry. Specifically, the base units or monomers which served for the construction of the hyperbranched polymer may be of different nature and their distribution is irregular. The branches of the polymer may be of different nature and lengths. The number of base units, or monomers, may be different according to the different branchings. While being asymmetric, hyperbranched polymers may have an extremely branched structure, around a core; successive generations or layers of branching; a layer of terminal chains.

Hyperbranched polymers are generally derived from the polycondensation of one or more monomers ABx, A and B being reactive groups that are capable of reacting together, x being an integer greater than or equal to 2, but other preparation processes may be envisaged.

Hyperbranched polymers are characterized by their degree of polymerization DP=1-b, b being the percentage of non-terminal functions of B which have not reacted with a group A. Since the condensation is not systematic, unlike for the synthesis of dendrimers (see hereinbelow), the degree of polymerization is less than 100%. A terminal group T on the hyperbranched polymer can be made to react to obtain a particular function at the end of chains.

Several hyperbranched polymers can be combined together, by covalent bonding or another type of bonding, by means of their terminal groups. Such polymers, which are said to be bridged, are included in the definition of the hyperbranched polymers according to the present invention.

Numerous hyperbranched polymers and dendrimers have already been described. Reference may be made, for example, to: D. A. Tomalia et al., Angew. Chem. (Engl. 29, 138-175 (1990); N. Ardoin and D. Astruc, Bull. Soc. Chim. Fr. 132, 875-909 (1995); B. I. Voit, Acta Polymer, 46, 87-99 (1995).

Such polymers are described in particular in B. I. Voit, Acta Polymer., 46, 87-99 (1995); EP-682 059; WO-96/14346; WO-96/14345; WO-96/12754. Several hyperbranched polymers can be combined together, by covalent bonding or another type of bonding, by means of their terminal groups.

Such polymers, which are said to be bridged, are included in the definition of the hyperbranched polymers according to the present invention.

“Dendrimers” are macromolecules consisting of monomers which associate by means of an arborescent process around a multifunctional central core.

Dendrimers thus have a fractal (or fractal molecule) structure, consisting of a core, a given number of generations of branches (or wedges), of internal cavities originating from said branches of the molecule, and of terminal functions.

Dendrimers are, structurally, highly branched polymers and oligomers having a well-defined chemical structure.

Dendrimers may be in the form of an assembly of molecules of the same generation, the assembly being referred to as “monodisperse”; they may also be in the form of assemblies of different generations, which are referred to as being “polydisperse”. The definition of dendrimers according to the present invention includes both monodisperse dendrimer assemblies and polydisperse dendrimer assemblies.

The generations of branches consist of structural units, which are identical for the same generation of branches and which may be identical or different for different generations of branches. All of the junction points of branches of the same generation are located at an equal distance from the core; this corresponds to a generation.

The generations of branches extend radially in a geometrical progression from the core. The terminal groups of an n^{th} generation dendrimer are the terminal functional groups of the branches of the n^{th} generation, referred to as the terminal generation.

The definition of dendrimers given above includes molecules bearing symmetrical branching; it also includes molecules bearing non-symmetrical branching, for instance dendrimers in which the branches are lysine groups, in which the branching of one generation of wedges on the preceding generation takes place on the α and ε amines of lysine, which leads to a difference in the length of the wedges of the various branches.

Dendrimers also known as “dense star polymers” or “starburst polymers” or “rod-shaped dendrimers” are included in the present definition of dendrimers. The molecules known as “arborols” and “cascade molecules” are also included in the definition of dendrimers according to the present invention.

Moreover, several dendrimers may be combined together, via a covalent bond or another type of bonding, by means of their terminal groups to give species known as “bridged dendrimers” or “dendrimer aggregates”. Such species are included in the definition of dendrimers according to the present invention.

Dendrimers may be in the form of an assembly of molecules of the same generation, the assembly being referred to as “monodisperse”; they may also be in the form of assemblies of different generations, which are referred to as being “polydisperse”. The definition of dendrimers according to the present invention includes both monodisperse dendrimer assemblies and polydisperse dendrimer assemblies.

According to another variant of the invention, the thiolated compound used according to the invention denotes a non-polymeric organic compound and may be represented by formula (XV):

W(SH)ₙ (XV)

in which:
n denotes an integer greater than or equal to 2, preferably between 2 and 10, preferably between 2 and 5, and
W denotes a linear or branched or (hetero)cyclic, saturated C₂ to C₈₀ multivalent (at least divalent) radical, an aromatic radical, or a heteroaromatic cyclic radical, W also possibly containing one or more heteroatoms such as O, N or S and/or one or more functions chosen from ester, ketone, amide and urea functions, preferably ester or ketone functions, and/or possibly being substituted with one or more linear or branched C₁-C₁₀ alkyl or linear or branched C₁-C₁₀ alkoxy groups, it being understood that when the radical W is substituted, the thiol functions may be borne by the substituent(s).

The term “cyclic radical” means a hydrocarbon-based or heterocyclic saturated monocyclic radical, a saturated or aromatic polycyclic radical, for example biphenyl, or fused rings, for instance a naphthyl radical.

The molar mass of the compounds of formula (XV) is generally between 90 and 1500 g·mol⁻¹.

According to a first embodiment, the compound bearing a thiol unit of formula (XV) is such that n=2 and W denotes a linear or branched C₂-C₂₀, preferably C₂-C₁₂ saturated divalent hydrocarbon-based radical.

According to this embodiment, the compound bearing a thiol unit denotes, for example, 1,2-ethanedithiol, 1,2-propanedithiol, 1,3-propanedithiol, 1,4-butanedithiol, 1,6-hexanedithiol, 1,7-heptanedithiol, 1,8-octanedithiol, 1,9-nonanedithiol, 1,10-decanedithiol, 1,12-dodecanedithiol, 2,2-dimethyl-1,3-propanedithiol, 3-methyl-1,5-pentanedithiol, or 2-methyl-1,8-octanedithiol.

According to another embodiment, the compound bearing a thiol unit of formula (XV) is such that n=3 and W denotes a linear or branched C₃-C₂₀, preferably linear or branched C₂-C₁₂ saturated trivalent hydrocarbon-based radical.

According to this other embodiment, the compound bearing a thiol unit may be chosen, for example, from 1,1,1-tris(mercaptomethyl)ethane, 2-ethyl-2-mercaptomethyl-1,3-propanedithiol, and 1,2,3-propanetrithiol.

According to a third embodiment, the compound bearing a thiol unit of formula (XV) is such that n=2 or 3 and W denotes a linear or branched C₃-C₂₀, preferably linear or branched C₂-C₁₂, saturated divalent or trivalent hydrocarbon-based radical, said radical containing one or more non-adjacent heteroatoms chosen from O and S.

According to this third embodiment, the compound bearing a thiol unit may be chosen, for example, from: C₂-C₁₂ bis(mercaptoalkyl) ethers and sulfides such as bis(2-mercaptoethyl) ether, bis(2-mercaptoethyl) sulfide, bis(2-mercaptoethylthio-3-mercaptopropane) sulfide, (C₁-C₅)bis(2-mercapto(C₁-C₃)alkylthio)alkanes or (C₁-C₅)bis(2-mercapto(C₁-C₃)alkylthio)mercaptoalkanes, for instance bis(2-mercaptoethylthio)methane, 1,2-bis(2-mercaptoethylthio)ethane, 1,3-bis(2-mercaptoethylthio)propane, 1,2-bis(2-mercaptoethylthio)propanethiol, 1,2-bis(2-mercaptoethyl)thio-3-mercaptopropane and 1,2,3-tris(2-mercaptoethylthio)propane.

Preferably, according to this embodiment, compound (XV) is chosen from 1,2-bis(2-mercaptoethylthio)propanethiol, 1,2,3-tris(2-mercaptoethylthio)propane and tetrakis(2-mercaptoethylthiomethyl)methane.

According to a fourth embodiment, the compound bearing a thiol unit of formula (XV) is such that n denotes an integer greater than or equal to 2 and W denotes a linear or branched C₃-C₂₀, preferably linear or branched C₂-C₁₂, hydrocarbon-based saturated multivalent (at least divalent) radical, said radical containing at least one ester function.

According to this fourth embodiment, the compound bearing a thiol unit may be chosen from: esters of polyols (glycols, triols, tetraols, pentaols, hexaols) and of C₁-C₆ mercaptocarboxylic acid, such as ethylene glycol bis(2-mercaptoacetate), ethylene glycol bis(3-mercaptopropionate), ethylene glycol bis(thioglycolate), trimethylolpropane tris(thioglycolate), trimethylolpropane tris(β-mercaptopropionate), pentaerythritol tetrakis(thioglycolate), pentaerythritol tetrakis(β-mercaptopropionate), dipentaerylthritol hexakis(β-mercaptoproprionate), trimethylolpropane tris(2-mercaptoacetate), trimethylolpropane tris(3-mercaptopropionate), pentaerythritol tetrakis(2-mercaptoacetate), pentaerythritol tetrakis(3-mercaptopropionate), pentaerythritol tetrakis(3-mercaptobutanate), and dipentaerythritol hex-3-mercaptopropionate.

Preferably, according to this fourth embodiment, the compound bearing a thiol unit is chosen from trimethylolpropane tris(2-mercaptoacetate), trimethylolpropane tris(3-mercaptopropionate), pentaerythritol tetrakis(2-mercaptoacetate), pentaerythritol tetrakis(3-mercaptopropionate), pentaerythritol tetrakis(3-mercaptobutanate) and dipentaerythritol hex-3-mercaptopropionate.

Particularly preferably, the compound bearing a thiol unit is pentaerythritol tetrakis(3-mercaptopropionate).

According to a fifth embodiment, the compound bearing a thiol unit, of formula (XV), is such that n=4 and W denotes a branched C₄-C₂₀, preferably C₈-C₁₄, hydrocarbon-based saturated tetravalent radical interrupted with one or more non-adjacent sulfur atoms.

According to this fifth embodiment, the compound bearing a thiol unit may be chosen from tetrakis(2-mercaptoethylthiomethyl)methane and bis(2-mercaptoethylthio-3-mercaptopropane) sulfide.

According to a sixth embodiment, the compound bearing a thiol unit, of formula (XV), is such that n=2 and W denotes a hydrocarbon-based cyclic divalent radical optionally containing one or more non-adjacent sulfur atoms, optionally substituted with one or more linear or branched C₁-C₁₀ alkyl radicals.

According to this sixth embodiment, the compound bearing a thiol unit may be chosen, for example, from 1,4-cyclohexanedithiol, 1,4-bis(mercaptomethyl)cyclohexane, 1,1-cyclohexanedithiol, 1,2-cyclohexanedithiol, 1,1-bis(mercaptomethyl)cyclohexane, and 2,5-dimercapto-1,4-dithiane.

According to a seventh embodiment, the compound bearing a thiol unit, of formula (XV), is such that n=3 and W denotes a substituted isocyanurate-type cyclic radical.

According to this seventh embodiment, the compound bearing a thiol unit may be chosen from polythiols of the isocyanurate class, described in U.S. Pat. No. 3,676,440 and US 2011 023 0585, such as tris((mercaptopropionyloxy)ethyl) isocyanurate.

According to this seventh embodiment, the compound bearing a thiol unit is preferably tris((mercaptopropionyloxy)ethyl) isocyanurate.

According to an eighth embodiment, the compound bearing a thiol unit, of formula (XV), is such that n=2 or 3 or 4 and W denotes an aromatic radical optionally substituted with one or more identical or different radicals of C₁-C₁₀ alkyl or C₁-C₁₀ alkoxy type, it being understood that, when the radical W is substituted, the thiol functions may be borne by the substituent(s).

According to this eighth embodiment, the compound bearing a thiol unit may be chosen, for example, from the following compounds:
1,2-dimercaptobenzene,
1,3-dimercaptobenzene,
1,4-dimercaptobenzene,
1,2-bis(mercaptomethyl)benzene,
1,3-bis(mercaptomethyl)benzene,
1,4-bis(mercaptomethyl)benzene,
1,2-bis(2-mercaptoethyl)benzene,
1,3-bis(2-mercaptoethyl)benzene,
1,4-bis(2-mercaptoethyl)benzene,
1,2-bis(2-mercaptoethyleneoxy)benzene,
1,3-bis(2-mercaptoethyleneoxy)benzene,
1,4-bis(2-mercaptoethyleneoxy)benzene,
1,2,3-trimercaptobenzene,
1,2,4-trimercaptobenzene,
1,3,5-trimercaptobenzene,
1,2,3-tris(mercaptomethyl)benzene,
1,2,4-tris(mercaptomethyl)benzene,
1,3,5-tris(mercaptomethyl)benzene,
1,2,3-tris(2-mercaptoethyl)benzene,
1,2,4-tris(2-mercaptoethyl)benzene,
1,3,5-tris(2-mercaptoethyl)benzene,
1,2,3-tris(2-mercaptoethyleneoxy)benzene,
1,2,4-tris(2-mercaptoethyleneoxy)benzene,
1,3,5-tris(2-mercaptoethyleneoxy)benzene,
1,2,3,4-tetramercaptobenzene,
1,2,3,5-tetramercaptobenzene,
1,2,4,5-tetramercaptobenzene,
1,2,3,4-tetrakis(mercaptomethyl)benzene,
1,2,3,5-tetrakis(mercaptomethyl)benzene,
1,2,4,5-tetrakis(mercaptomethyl)benzene,
1,2,3,4-tetrakis(2-mercaptoethyl)benzene,
1,2,3,5-tetrakis(2-mercaptoethyl)benzene,
1,2,4,5-tetrakis(2-mercaptoethyl)benzene,
1,2,3,4-tetrakis(2-mercaptoethyleneoxy)benzene,
1,2,3,5-tetrakis(2-mercaptoethyleneoxy)benzene,
1,2,4,5-tetrakis(2-mercaptoethyleneoxy)benzene,
2,2′-dimercaptobiphenyl,
4,4′-dimercaptobiphenyl,
4,4′-dimercaptobibenzyl,
2,5-toluenedithiol,
3,4-toluenedithiol,
1,4-naphthalenedithiol,
1,5-naphthalenedithiol,
2,6-naphthalenedithiol,
2,7-naphthalenedithiol,
2,4-dimethylbenzene-1,3-dithiol,
4,5-dimethylbenzene-1,3-dithiol,
9,10-anthracenedimethanethiol,
1,3-bis(2-mercaptoethylthio)benzene,
1,4-bis(2-mercaptoethylthio)benzene,
1,2-bis(2-mercaptoethylthiomethyl)benzene,
1,3-bis(2-mercaptoethylthiomethyl)benzene,
1,4-bis(2-mercaptoethylthiomethyl)benzene,
1,2,3-tris(2-mercaptoethylthio)benzene,
1,2,4-tris(2-mercaptoethylthio)benzene,
1,3,5-tris(2-mercaptoethylthio)benzene,
1,2,3,4-tetrakis(2-mercaptoethylthio)benzene,
1,2,3,5-tetrakis(2-mercaptoethylthio)benzene,
1,2,4,5-tetrakis(2-mercaptoethylthio)benzene, and
3,4-thiophenedithiol.

According to this eighth embodiment, compound (XV) is chosen from 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(2-mercaptoethyl)benzene, 1,2,4-tris(2-mercaptoethyl)benzene, 1,3,5-tris(2-mercaptoethyl)benzene, 1,2,3-tris(2-mercaptoethyleneoxy)benzene, 1,2,4-tris(2-mercaptoethyleneoxy)benzene, 1,3,5-tris(2-mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(2-mercaptoethyl)benzene, 1,2,3,5-tetrakis(2-mercaptoethyl)benzene, 1,2,4,5-tetrakis(2-mercaptoethyl)benzene, 1,2,3,4-tetrakis(2-mercaptoethyleneoxy)benzene, 1,2,3,5-tetrakis(2-mercaptoethyleneoxy)benzene, 1,2,4,5-tetrakis(2-mercaptoethyleneoxy)benzene, 1,2,3-tris(2-mercaptoethylthio)benzene, 1,2,4-tris(2-mercaptoethylthio)benzene, 1,3,5-tris(2-mercaptoethylthio)benzene, 1,2,3,4-tetrakis(2-mercaptoethylthio)benzene, 1,2,3,5-tetrakis(2-mercaptoethylthio)benzene, 1,2,4,5-tetrakis(2-mercaptoethylthio)benzene and 3,4-thiophenedithiol.

According to a ninth embodiment, the compound bearing a thiol unit, of formula (XV), is such that n=2 or 3 or 4 and W denotes a fatty acid triglyceride or a plant oil, which are optionally substituted, it being understood that when the radical W is substituted, the thiol functions may be borne by the substituent(s).

According to another particular embodiment of the invention, the thiolated and/or hydroxylated compound is chosen from thiolated hydroxylated and/or thiolated fatty acid triglyceride derivatives, such as those of formula (XVI):

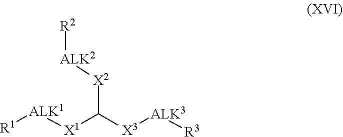

in which:
R¹, R² and R³, which may be identical or different, represent a hydrogen atom or a hydroxyl or thiol group, preferably a thiol group;
ALK¹, ALK² and ALK³, which may be identical or different, represent a (C₁-C₃₀)alkylene group optionally substituted with one or more hydroxyl or thiol groups, preferably thiol groups;
X¹, X² and X³, which may be identical or different, preferably identical, represent a group —C(Y)—Y′— or —Y′—C(Y)— with Y and Y′, which may be identical or different, preferably identical, representing a heteroatom such as O, S and N, preferably 0.

Preferably, the compounds of formula (XVI) are such that:
R¹, R² and R³ represent a hydrogen atom;
ALK¹ represents a (C₁₀-C₂₄)alkylene and particularly (C₁₄-C₂₀)alkylene group, which is preferably linear;
ALK² represents a (C₁₀-C₂₄)alkylene and particularly (C₁₄-C₂₀)alkylene group, which is preferably linear, substituted with one or more thiol groups;
ALK³ represents a (C₁₀-C₂₄)alkylene and particularly (C₁₄-C₂₀)alkylene group, which is preferably linear, substituted with one or more thiol groups, preferably two thiol groups; and/or
X¹, X² and X³ are identical and represent a —C(O)—O— or —O—C(O)— group.

According to this embodiment, the compound bearing a thiol unit may be chosen, for example, from: fatty acid triglycerides or plant oils modified with thiol groups by chemical reaction, for instance thiolated soybean oils and hydroxylated and thiolated soybean oils, notably the Polymercaptan® products from the company Chevron Phillips, such as Polymercaptan 407 (mercapto hydroxy soybean oil) and Polymercaptan 358 (mercaptanized soybean oil) of formula (XVII) below:

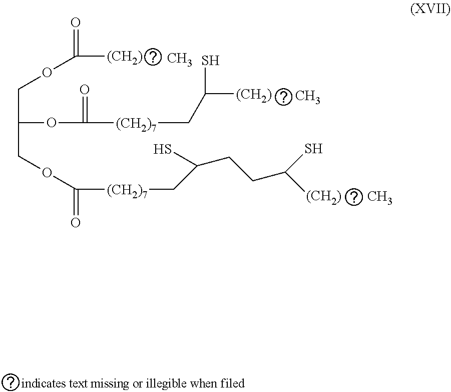

More preferentially, the thiolated hydroxylated and/or thiolated fatty acid triglyceride derivatives of formula (XVI) are such as those of formula (XVII) above.

According to a particular embodiment of the invention, the thiolated and/or hydroxylated compound is chosen from polyhydroxylated, polythiolated, or (poly)hydroxylated and (poly)thiolated compounds containing several hydroxyl and/or thiol groups, and having a weight-average molecular weight ranging from 500 to 1 000 000 g·mol⁻¹, preferably ranging from 500 to 500 000 g·mol⁻¹, and preferentially ranging from 500 to 100 000 g·mol⁻¹.

According to this variant, preference will be given to the compounds of formula (XV) for which n denotes an integer greater than or equal to 3, between 3 and 10 and more preferentially between 3 and 5.

Preferably, according to this variant, the compounds of formula (XV) are chosen from compounds of the second embodiment, or from compounds of the third embodiment; or from compounds of the fourth embodiment, in particular such as trimethylolpropane tris(2-mercaptoacetate), trimethylolpropane tris(3-mercaptopropionate), pentaerythrityl tetrakis(2-mercaptoacetate), pentaerythrityl tetrakis(3-mercaptopropionate), pentaerythrityl tetrakis(3-mercaptobutanate) or dipentaerythrityl hex-3-mercaptopropionate, or from compounds of the fifth embodiment, or from compounds of the seventh embodiment, in particular such as tris((mercaptopropionyloxy)ethyl) isocyanurate.

Particularly preferably, according to this variant, the compounds of formula (XV) are chosen from trimethylolpropane tris(2-mercaptoacetate), trimethylolpropane tris(3-mercaptopropionate), pentaerythrityl tetrakis(2-mercaptoacetate), pentaerythrityl tetrakis(3-mercaptopropionate), pentaerythrityl tetrakis(3-mercaptobutanate), dipentaerythrityl hex-3-mercaptopropionate or tris((mercaptopropionyloxy)ethyl) isocyanurate.

According to another variant, the compound bearing a thiol unit according to the invention denotes a polymeric compound and may be represented by formula (XVIII):

POL(SH)ₙ (XVIII)

in which:
n denotes an integer greater than or equal to 5, preferably between 5 and 5000, preferably from 5 to 1000, and
POL denotes a carbon-based or silicon-based multivalent (at least pentavalent) polymeric radical, POL also possibly containing one or more heteroatoms such as O, N or S, and/or one or more functions chosen from ester, ketone, amide, urea and carbamate functions, and/or possibly being substituted with one or more linear or branched C₁-C₁₀ alkyl or linear or branched C₁-C₁₀ alkoxy groups, it being understood that when POL is substituted, the thiol functions may be borne by the substituent(s).

The molar mass of the compounds of formula (XVIII) is generally between 500 and 400 000 g·mol⁻¹ and preferably between 500 and 150 000 g·mol⁻¹.

In a particular embodiment, POL denotes a multivalent homopolymer or copolymer radical.

In a particular embodiment, POL denotes a polymeric radical of star, comb, brush or dendritic type.

The POL radical may be of natural origin (such as polysaccharides, peptides) or synthetic origin (such as acrylic polymers, polyesters, polyglycols).

The thiol functions (—SH) may be terminal and/or side groups.

According to a first embodiment, the thiolated compound of formula (XVIII) is such that POL denotes a hydrocarbon-based polymeric radical.

Examples that may be mentioned include the polymers described in the following articles: Polymers containing groups of biological activity, C. G. Overberger et al., Polytechnic Institute of Brooklyn, http://pac.iupac.org/publications/pac/pdf/1962/pdf/0402x0521.pdf and Mercaptan-containing polymers, Advances in Polymer Science, volume 15, 1974, pages 61-90.

In particular, mention may be made of compounds bearing a thiol unit, of formula (XVIII), such as poly(vinyl mercaptan), poly(4-mercaptostyrene), poly(vinylbenzyl mercaptan), poly(4-mercaptostyrene)-co-poly(methyl methacrylate), and also polymers containing amide functions, such as poly(thiolated hexamethyleneadipamide).

The compounds of formula (XVIII) also denote proteins and peptides with thiol units, for instance the structures represented in the following table:

TABLE 1

| | |
|---|---|
| 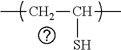 | 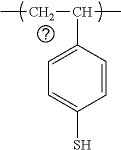 |
| 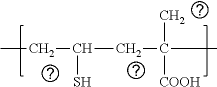 | 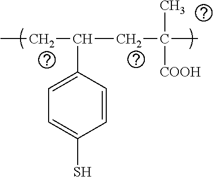 |
| 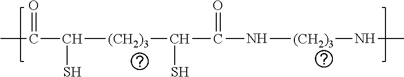 | 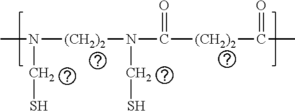 |
| 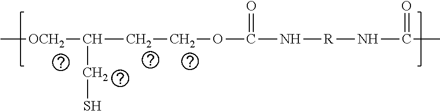 | 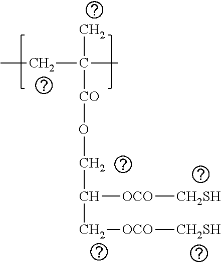 |
| 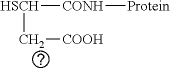 | 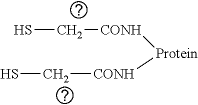 |

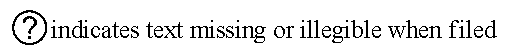

The thiol compounds of formula (XVIII) also denote the compounds of formula (XVIII) such that POL denotes a radical termed dendrimer or polymer which is branched or hyperbranched, and the thiol groups are end groups. As examples, mention may be made of the polymers described in the article Progress in Organic Coatings, volume 63, issue 1, July 2008, pages 100-109.

As an example of a synthesis of such polymers, mention may be made of the synthesis described in said article in which the polymer Boltorn H40 is transformed into a thiol polymer of formula (XVIII) according to the scheme below:

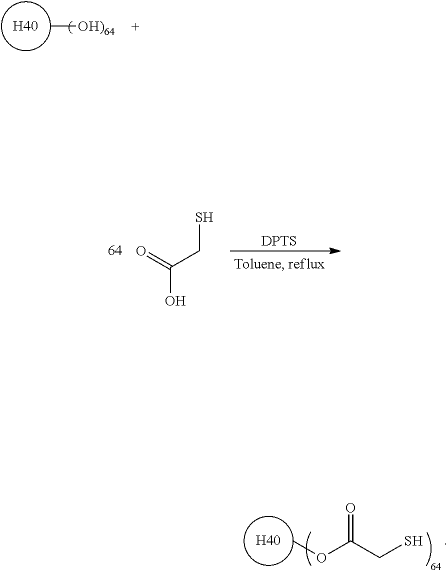

The structure of the thiol polymer (XVIII) obtained is given below:

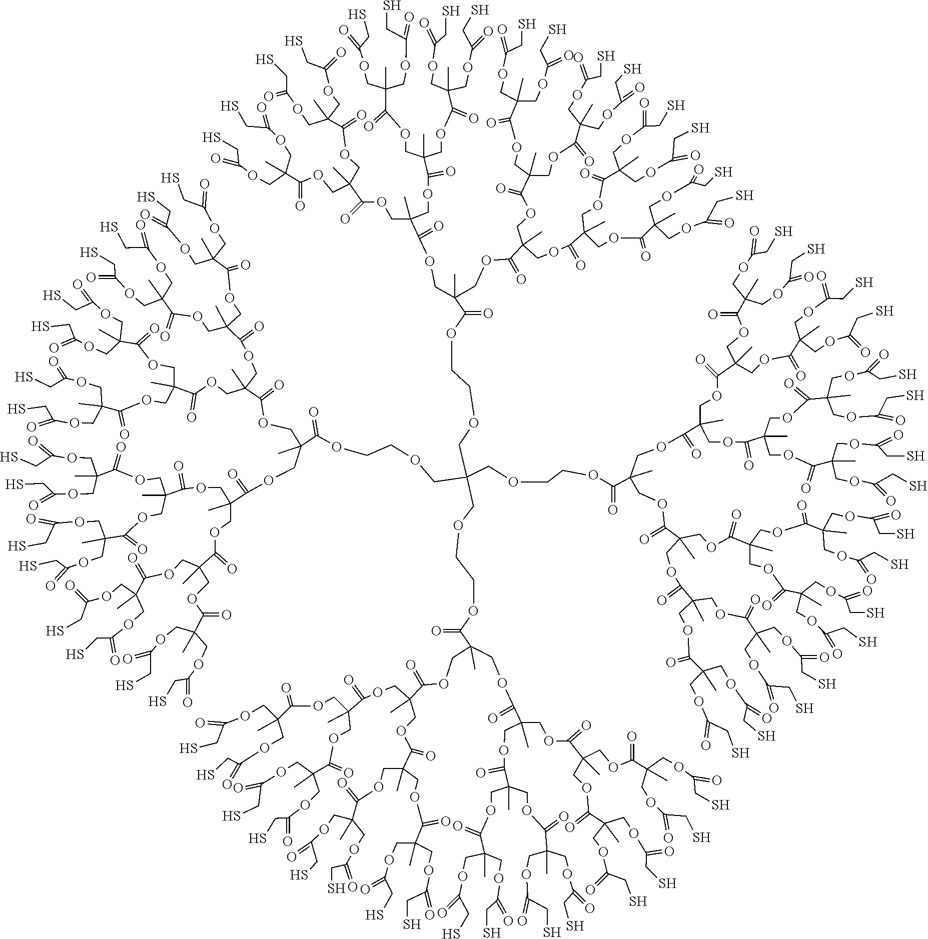

The compound bearing a thiol unit, of formula (XVIII), may also denote a hyperbranched or dendritic polymer modified with thiol functions, as described in patent application FR 2 761 691.

As examples of hyperbranched polymers and dendrimers, mention may be made of the compounds including thiol functional groups of formula (XIX) below:

HS-A-C(Y)—X— (XIX)

in which:
Y represents an oxygen or sulfur atom or a group NR′;
X represents i) an oxygen atom or ii) a group —N(R′)— in which R′ is chosen from a) a hydrogen atom, b) a linear or branched, saturated or unsaturated C₁-C₆ alkyl group, c) a linear or branched, saturated or unsaturated C₁-C₆ monohydroxyalkyl or polyhydroxyalkyl group, d) a C₁-C₆ aminoalkyl group or a polyalkyleneimine group; preferably, X represents —N(R′)— with R′ representing a hydrogen atom or a (C₁-C₄)alkyl group such as methyl; and
A represents a linear, branched or cyclic, saturated or unsaturated (C₁-C₁₂)alkylene group; this group being optionally interrupted with one or more heteroatoms such as O, S or N and/or optionally substituted with one or more groups chosen from amino (—NH₂), acylamino (—N(H)—C(O)—R) or aminoacyl (RN(H)—C(O)—) in which R represents a linear, branched or cyclic, saturated or unsaturated C₁-C₁₀ alkyl, carboxyl (—C(O)OH) or ester (—C(O)—OR) group in which R represents a linear, branched or cyclic, saturated or unsaturated C₁-C₁₀ alkyl group.

Preferably, the thiolated polymers according to the invention are chosen from hyperbranched polymers, and notably polyethyleneimine including at least one group chosen from the groups of formula (XIX) as defined previously.

Preferably, Y represents an oxygen atom. Preferably, the heteroatoms are chosen from oxygen and nitrogen (O and N).

Preferably, A is a methylene, ethylene, propylene, methylpropylene, ethylpropylene, tetramethylene, pentamethylene, hexamethylene or phenylene group.

Advantageously, A represents a radical corresponding to one of the formulae (a) to (d) below:
(a) —CHR¹—CHR²—CHR³—
(b) —CHR′¹—CHR′²CHR′³—CHR′⁴—
(c)

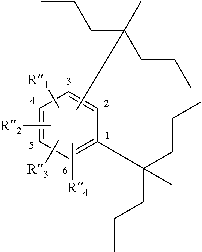

(d) —(CHR′″¹)ₖ—(CHR′″²)—CH(CO₂H)—NH—
   in which formulae (a), (b), (c) and (d):
R¹, R², R³, R′¹, R′², R′³ and R′⁴, R′″¹ and R′″², which may be identical or different, represent: a hydrogen atom; a linear, branched or cyclic, saturated or unsaturated C₁-C₆ alkyl radical; an amino radical (—NH₂); a carboxylic acid radical (—COOH); a C₁-C₁₀ alkylamino radical; a C₁-C₁₀ acylamino radical;
R″¹, R″², R″³ and R″⁴, which may be identical or different, represent a hydrogen atom or a linear or branched, saturated or unsaturated C₁-C₄ alkyl group; the arrows indicating the positions of the substitutions; and
k is an integer, preferentially 0 or 1;

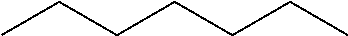

represents the point of attachment to the rest of the molecule on the phenylene group in position 1-2, or 1-3, or 1-4; it being understood that the radicals R″₁, R″₂, R″₃ and R″₄ are then positioned on the carbon atoms 3, 4, 5, 6, or 2, 4, 5 or 6 or 2, 3, 5, 6, respectively.

According to a preferred embodiment of the invention, the thiolated polymers are hyperbranched polymers and dendrimers including functional groups of formula (XIX) such that A is chosen from:
—CH₂—CH(CO₂H)—NH— and Y represents an oxygen atom;
—(CH₂)₂—(CH₃CONH)CH— and Y represents an oxygen atom;
—(CH₂)₃— and Y represents an oxygen atom or an NH group.

In particular, A is the propylene group —CH₂—CH₂—CH₂— and Y represents an oxygen atom, the group of formula (XIX) then corresponding to formula (XX) below:

HS—CH₂—CH₂—CH₂—C(O)—X— (XX)

in which X is as defined in formula (XIX); preferably, X represents —N(R′)— with R′ representing a hydrogen atom or a (C₁-C₄)alkyl group such as methyl.

Preferentially, in formulae (XIX) and (XX), X is chosen from an oxygen atom and an NH group.

According to one of the preferred embodiments of the invention, the thiol polymers are as described in FR 2 853 533, that is to say poly-N-α- and N-ε-lysine and ornithine of formula I, bearing a thiol function, which may be obtained from poly-N-α- and N-ε-lysine and omithine by reaction with a thiolactone, for instance thiobutyrolactone (dihydrothiophen-2(3H)-one).

According to a preferred embodiment of the invention, the hyperbranched polymers and dendrimers that are useful in the invention include functional groups corresponding to formula (XXI):

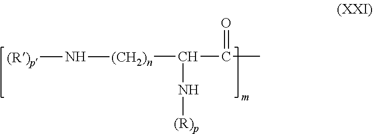

in which:
p is different from p′ and p and p′ are equal to 0 or 1;
n is 3 or 4;
if p′ is equal to 0, then the neighboring NH is engaged in an N-ε polymerization;
if p is equal to 0, then the neighboring NH is engaged in an N-α polymerization;
if p or p′ is equal to 1, then R or R′ represents —B—SH, with B representing a saturated or unsaturated, linear or branched C₁-C₃₀ hydrocarbon-based chain which may be interrupted with one or more heteroatoms or groups, alone or in combination, such as: —N(R′)—, —O—, —S(O)ᵣ—, —C(O)—, —C(S)— or —C(NR¹)—, with r being equal to 0, 1 or 2, and/or with one or more 5-, 6- or 7-membered aryl, heteroaryl, cycloalkyl or heterocycloalkyl which may be substituted with one or more halogen atoms or groups from among: hydroxyl, amino, carboxyl, (di)(C₁-C₈)alkylamino, (C₁-C₈)acylamino, (C₁-C₈)acyloxy, (C₁-C₈)alkyloxycarbonylamino, (C₁-C₈)alkylaminocarbonyloxy and (C₁-C₈)alkylaminocarbonyl;
given that R or R′ may also, in part only, represent a hydrogen atom,
and/or —C(NH)— and salts thereof,
and/or —C(NH)—N(H)—C(NH)—NH₂ and salts thereof,
R₁ represents a hydrogen atom or a (C₁-C₈)alkyl, (C₁-C₈)acyl, (C₁-C₈)alkyloxycarbonyl, (C₁-C₈)alkylaminocarbonyl or halo group;
B may also represent an optionally substituted 5-, 6- or 7-membered aryl, heteroaryl, cycloalkyl or heterocycloalkyl group;
m represents an integer ranging from 3 to 10 000.

Preferably, the degree of thiol function grafting is greater than or equal to 1%.

Advantageously, the poly N-α- and N-ε-lysine and ornithine corresponding to formula (XXI) have: 5<m<1000.

The term “theoretical degree of thiol function grafting” represents the theoretical percentage of lysine or ornithine units bearing the thiol function in the compound of formula (XXI).

Examples of hyperbranched polymers that may be mentioned most particularly include hyperbranched thiolated polyethyleneimines, such as those described in patent application EP 103 759 with a molecular molar mass ranging from 30×10⁴ to 50×10⁴ g·mol⁻¹.

These polymers are prepared according to methods that are conventional to those skilled in the art, such as the methods described in French patent application FR 2 761 691 and EP 1 037 938.

According to a particular embodiment of the invention, the branched or hyperbranched polymer(s) and dendrimer(s) bear thiol terminal groups, such as the Boltorn™ dendritic polythiols from the company BASF esterified with compounds such as thioglycolic acid and described in the literature.

Polymers such as polypropylene ether glycol bis(β-mercaptopropionate) may also be used according to the invention. They are prepared via the methods known to those skilled in the art. Mention may be made, for example, of the preparation method by esterification reaction of polypropylene ether glycol such as Pluracol P201 sold by the company Wyandotte Chemical Corp. and β-mercaptopropionic acid.

According to a particular embodiment of the invention, the hydroxylated and/or thiolated polymers are polyethoxylated of formula (XXII), and also the optical isomers thereof, the acid or base salts thereof, and the solvates thereof such as hydrates:

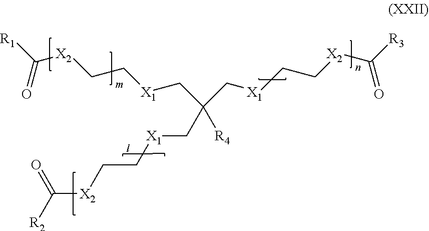

in which:
R₁, R₂ and R₃, which may be identical or different, preferably identical, represent a hydroxy(C₁-C₆)alkyl or thio(C₁-C₆)alkyl group, preferably a thio(C₁-C₆)alkyl group;
R₄ represents a hydrogen atom or a group from among: hydroxyl, thiol, amino or (C₁-C₆)alkyl, preferably (C₁-C₄)alkyl such as ethyl;
X₁ and X₂, which may be identical or different, preferably identical, represent an oxygen or sulfur atom, or amino, preferably oxygen;
m, n and 1, which may be identical or different, represent an integer greater than or equal to 1.

The thiol polymer compounds of formula (XXII) are commercially available. Mention may be made, for example, of the products Thiocure® from the company Bruno Brock, Thiocure® ETTMP 1300 (Ethoxylated-Trimethylolpropane Tri-3-Mercaptopropionate (CAS #345352-19-4) and Thiocure® ETTMP 700 (Ethoxylated-Trimethylolpropane Tri-3-Mercaptopropionate (CAS #345352-19-4).

According to a preferred embodiment, the crosslinking agent R is a (poly)thiolated and/or (poly)hydroxylated compound, in particular chosen from non-polymeric (poly)thiolated and/or (poly)hydroxylated compounds such as polyhydroxylated compounds (liposoluble polyol), polythiolated compounds (dithiol compounds), hydroxylated and/or thiolated alkoxysiloxanes, silicas functionalized with radicals, notably of the alkyl type, substituted with thiol functions, latex bearing thiol groups, particles coated with compound(s) bearing at least two thiol functions and polymeric (poly)thiolated and/or (poly)hydroxylated compounds such as homopolymers, copolymers, star, comb, brush and dendritic compounds with hydroxyl and/or thiol units, which are preferably organic or silicon-based.

Preferably, the polymeric (poly)thiolated and/or (poly)hydroxylated compounds are chosen from (di)ol polymers, in particular polyolefin (poly)ols, polydi(C₁-C₆)alkylsiloxane (poly)ols, polyester (poly)ols, hydroxylated thiolated and/or thiolated fatty acid triglyceride derivatives, amine thiols derived from dendrimers or polyethyleneimines (PEI) and silicone thiols.

Preferably, the polymeric (poly)thiolated and/or (poly)hydroxylated compounds are chosen from polydimethylsiloxane diols such as hydroxy-terminated polydimethylsiloxanes; thiolated poly(C₁-C₄)alkylsiloxanes such as polydimethylsiloxanes bearing at least two thiol groups; and fatty acid triglycerides or plant oils modified with thiol groups by chemical reaction.

Preferably, the (poly)thiolated and/or (poly)hydroxylated compounds used according to the invention are chosen from liposoluble polyols, dithiol compounds, hydroxylated and/or thiolated alkoxysiloxanes, silicas functionalized with radicals, notably of the alkyl type, substituted with thiol functions, latex bearing thiol groups, particles coated with compound(s) bearing at least two thiol functions, polyolefin (poly)ols, polydi(C₁-C₆)alkylsiloxane (poly)ols, polyester (poly)ols, amino thiols derived from dendrimers or polyethylenimines (PEI), silicone thiols, polydimethylsiloxane diols, poly(C₁-C₄)alkylsiloxane thiols and fatty acid triglycerides or plant oils modified with thiol groups by chemical reaction.

Preferably, the (poly)thiolated and/or (poly)hydroxylated compounds used according to the invention are chosen from polydimethylsiloxanes including at least two thiol groups.

### (Poly)carbonyl Crosslinking Agents

According to a particular embodiment, the crosslinking agent R is a (poly)carbonyl compound.

In particular, the (poly)carbonyl compound is chosen from terephthalaldehyde, 5,5-dimethyl-1,3-cyclohexanedione, phenylglyoxal, isophthalaldehyde, 4-acetylbenzaldehyde, 4,4-diformyltriphenylamine, 2-acetylbenzaldehyde, 3-(2-furoyl)quinoline-2-carboxaldehyde 3-(2-furoyl)quinoline-2-carboxaldehyde, 3-acetylbenzaldehyde, 9-(2-ethylhexyl)carbazole-3,6-dicarboxaldehyde, phthaldialdehyde, 1,3-cyclohexanedione, 4,4′-biphenyldicarboxaldehyde, benzene-1,3,5-tricarboxaldehyde, and oxidized inulin.

In particular, the (poly)carbonyl compound is chosen from terephthalaldehyde, 5,5-dimethyl-1,3-cyclohexanedione, phenylglyoxal, isophthalaldehyde, 4-acetylbenzaldehyde, 4,4-diformyltriphenylamine, 2-acetylbenzaldehyde, 3-(2-furoyl)quinoline-2-carboxaldehyde, 3-(2-furoyl)quinoline-2-carboxaldehyde, 3-acetylbenzaldehyde, 9-(2-ethylhexyl)carbazole-3,6-dicarboxaldehyde, phthaldialdehyde, 1,3-cyclohexanedione, 4,4′-biphenyldicarboxaldehyde, benzene-1,3,5-tricarboxaldehyde, oxidized inulin, and terephthalaldehyde, preferably terephthalaledhyde.

According to this embodiment, the (poly)carbonyl compound is associated in its implementation with an amine catalyst as described, for example, in the articles Progress in coating 129, 21-25 (2019) and Progress in coating 135, 510-516 (2019); preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

### (Poly)acrylate Crosslinking Agents

According to a particular embodiment, the crosslinking agent R is a (poly)acrylate compound.

More particularly, the (poly)acrylate compound may be chosen from 1,3-butanediol diacrylate, 1,4-butanediol diacrylate, di(trimethylolpropane) tetraacrylate, glyceryl 1,3-diglycerolate diacrylate, glyceryl propoxylate (1PO/OH) triacrylate, 1,6-hexanediol diacrylate, 1,6-hexanediol ethoxylate diacrylate, hydroxypivalyl hydroxypivalate, neopentyl glycol diacrylate, neopentyl glycol propoxylate (1PO/OH) diacrylate, pentaerythritol tetraacrylate, pentaerythritol triacrylate, poly(propylene glycol) diacrylate, tricyclo[5.2.1.0^{2,6}]decanedimethanol diacrylate, trimethylolpropane ethoxylate (1EO/OH) methyl ether diacrylate, trimethylolpropane propoxylate triacrylate, trimethylolpropane triacrylate, tri(propylene glycol) diacrylate, tris[2-(acryloyloxy)ethyl] isocyanurate, N,N′-methylenebis(acrylamide), trimethylolpropane triacrylate, methylenebis(acrylamide), or mixtures of these compounds.

More particularly, the (poly)acrylate compound is trimethylolpropane triacrylate.

According to this embodiment, the (poly)acrylate compound is associated in its implementation with at least one amine catalyst as described, for example, in the articles Progress in coating 129, 21-25 (2019) and Progress in coating 135, 510-516 (2019); preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and preferably the catalyst is DBU (1,8-diazabicyclo[5.4.0]undec-7-ene).

### Metal Salt Crosslinking Agents:

According to another particular embodiment, the crosslinking agent R is a metal salt chosen from alkali metal salts, alkaline-earth metal salts such as magnesium salts, transition metal salts, post-transition metal salts such as aluminum or tin salts, metalloid salts such as boron salts, hydrates thereof and mixtures thereof.

Preferably, the metal salt(s) are chosen from post-transition metal salts such as aluminum salts, hydrates thereof and mixtures thereof.

The term “metal salt” means a salt resulting notably from the action of an acid on a metal, in particular a transition metal, post-transition metal, metalloid, alkali metal or alkaline-earth metal.

The metal salt(s) may be in the form of hydrates.

The metal salt(s) may be organic or inorganic.

The term “organic metal salt” means a salt resulting notably from the action of an organic acid on a metal, in particular transition metals, post-transition metals, metalloids, alkali metals or alkaline-earth metals, preferably resulting from the action of a carboxylic acid on a metal.

Preferably the metal salt(s) are chosen from organic metal salts, hydrates thereof and mixtures thereof.

The term “inorganic metal salt” means a salt resulting notably from the action of an inorganic acid on a metal, in particular a transition metal, a post-transition metal, a metalloid, an alkali metal or an alkaline-earth metal.

The term “inorganic acid” means an acid which does not include any carbon atoms, apart from carbonic acid.

According to a particular embodiment of the invention, the inorganic metal salt(s) may be chosen from halides such as chlorides, fluorides, iodides and bromides, carbonates, sulfates, phosphates, nitrates, perchlorates, hydrates thereof, and mixtures thereof.

Metal (poly)(hydroxy)(C₁-C₆)alkyl Carboxylate Crosslinking Agents

According to a more particular embodiment, the crosslinking agent R is an organic metal salt derived from a carboxylic acid.

More particularly, the crosslinking agent R is an organic metal salt chosen from metal (poly)(hydroxy)(C₁-C₆)alkylcarboxylates of alkali metals, alkaline-earth metals, transition metals, and post-transition metals such as aluminum.

It is understood that the metal (poly)(hydroxy)(C₁-C₆)alkylcarboxylate means that the (C₁-C₆)alkyl group is optionally substituted with one or more hydroxyl groups and one or more carboxyl or carboxylate groups. Preferably, the metal (poly)(hydroxy)(C₁-C₆)alkylcarboxylate represents R^{a}—C(O)—OM with M representing a transition metal such as titanium (Ti), or else a post-transition metal such as aluminum (Al), and R^{a} represents a linear or branched (C₁-C₆)alkyl group optionally substituted with at least one hydroxyl group.

According to a preferred embodiment of the invention, the metal salt(s) are organic, preferably chosen from citrates, lactates, glycolates, gluconates, acetates, propionates, fumarates, oxalates, glycinates and tartrates, hydrates thereof, and mixtures thereof, more preferentially acetates, lactates or mixtures thereof such as aluminum acetate or aluminum lactate.

According to a preferred embodiment, the metal salt(s) are chosen from basic aluminum acetate, aluminum oxalate, hydrated or non-hydrated aluminum citrate, aluminum lactate and aluminum glycinate, and mixtures thereof.

According to an even more preferred embodiment, the metal salt is basic aluminum acetate.

### Metal Alkoxide Crosslinking Agents

According to another particular embodiment, the crosslinking agent R is a compound chosen from the metal alkoxides of formulae (XXIIIₐ), (XXIII_{b}), (XXIII_{c}) and (XXIV_{d}) below and mixtures thereof:

M-(OR₁)ₙ (XXIIIₐ)

R-M-(OR₁)ₙ₋₁ (XXIII_{b})

(R₁O)ₙ₋₁-M-R″-M′-(OR₁′)_{n′-1} (XXIII_{c})

R-M(R′)—(OR₁)ₙ₋₂ (XXIII_{d})

in which formulae (XXIIIₐ), (XXIII_{b}), (XXIII_{c}) and (XXIII_{d}):
M and M′, which may be identical or different, represent an atom chosen from alkaline-earth metals, transition metals, metals of the lanthanide family, post-transition metals such as aluminum or tin and metalloids such as boron; preferably transition metals such as Ti and post-transition metals such as aluminum;
n and n′ respectively represent the valencies of the atoms represented by M and M′;
R₁ and R′₁, which may be identical or different, represent a linear or branched, saturated or unsaturated hydrocarbon-based group containing from 1 to 30 carbon atoms, preferably from 1 to 6 carbon atoms, said hydrocarbon-based group being optionally interrupted with 1 to 20 heteroatoms chosen from O, N, S and P, notably O or N; and/or said hydrocarbon-based group being optionally substituted with one or more hydroxyl or carbonyl groups;
R and R′, which may be identical or different, represent a hydrogen atom or a linear, branched or cyclic, saturated or unsaturated hydrocarbon-based group containing from 1 to 30 carbon atoms, preferably from 2 to 20 carbon atoms, optionally interrupted with 1 to 20 heteroatoms chosen from O, N, S and/or P, notably O or N, and/or said hydrocarbon-based group being optionally substituted with one or more hydroxyl or carbonyl groups;
R″ represents —O—, —NR₂—, —S— or a linear, cyclic or branched, saturated or unsaturated divalent hydrocarbon-based group containing from 1 to 30 carbon atoms, preferably from 2 to 20 carbon atoms, optionally interrupted with 1 to 20 heteroatoms chosen from O, N, S and P, notably O or N, with R₂ representing a linear, cyclic or branched, saturated or unsaturated hydrocarbon-based group containing from 1 to 30 carbon atoms, preferably from 2 to 20 carbon atoms;

Preferably, M and M′, which may be identical or different, represent an atom chosen from transition metals such as titanium or zirconium or alkaline-earth metals such as magnesium, more preferentially chosen from transition metals such as titanium or zirconium, even more preferentially titanium.

Preferably, the organometallic compound(s) are chosen from the alkoxides of formula (XXIIIₐ) as defined previously.

According to a preferred embodiment, the organometallic compound(s) are chosen from the alkoxides of formula (XXIIIₐ), in which:
M represents an atom chosen from transition metals, metals of the lanthanide family, post-transition metals such as aluminum, tin, metalloids such as boron, or alkaline-earth metals such as magnesium or calcium;
n represents the valency of the atom represented by M;
R₁ represents a linear or branched saturated hydrocarbon-based group containing from 1 to 30 carbon atoms, preferably from 1 to 6 carbon atoms.

According to a more preferred embodiment, the organometallic compound(s) are chosen from the alkoxides of formula (XXIIIₐ) in which:
M represents an atom chosen from transition metals such as zirconium or titanium, metals of the lanthanide family, post-transition metals such as aluminum, tin, metalloids such as boron, and alkaline-earth metals such as magnesium, preferably M represents a titanium atom;
n represents the valency of the atom represented by M, notably 1, 2, 3 or 4, in particular 4;
R₁ represents a methyl, ethyl, 2-ethylhexyl, propyl, isopropyl, n-butyl, isobutyl or t-butyl group.

According to an even more preferred embodiment, the organometallic compound(s) are chosen from zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), and mixtures thereof.

Particularly preferably, the organometallic compound(s) are chosen from zirconium propoxide (Zr(OCH₂CH₂CH₃)₄), titanium propoxide (Ti(OCH₂CH₂CH₃)₄), titanium butoxide (Ti(OCH₂CH₂CH₂CH₃)₄) and mixtures thereof.

More preferentially, the crosslinking agent R is a compound of formula (XXIIIₐ) in which M represents an atom chosen from transition metals, notably titanium such as titanium butoxide.

### Crosslinking Agents Composed of a Metal Belonging to the Group of the Rare-Earth Metals

According to another particular embodiment, the crosslinking agent R is a compound of a metal belonging to the group of the rare-earth metals M″, and notably a salt of a metal belonging to the group of the rare-earth metals.

The term “salt of a metal belonging to the group of the rare-earth metals” means a salt notably derived from the action of an acid on a metal belonging to the group of the rare-earth metals.

The compound(s) of a metal belonging to the group of the rare-earth metals may be in the form of hydrates.

The compound(s) of a metal belonging to the group of the rare-earth metals may be organic or mineral. They may or may not be in salt form.

The term “organic salt of a metal belonging to the rare-earth metal group” means a salt notably derived from the action of an organic acid (notably a carboxylic acid) on a metal belonging to the group of the rare-earth metals.

The term “mineral salt of a metal belonging to the group of the rare-earth metals” means a salt notably derived from the action of a mineral acid on a metal belonging to the group of the rare-earth metals.

The term “inorganic acid” means an acid which does not include any carbon atoms, apart from carbonic acid.

As examples of metals belonging to the group of the rare-earth metals M″, mention may be made of scandium, yttrium, lanthanum, cerium, praseodymium, neodymium, promethium, samarium, europium, gadolinium, terbium, dysprosium, holmium, erbium, thulium, ytterbium and lutetium. Preferably, the metal(s) belonging to the group of the rare-earth metals are chosen from cerium, yttrium, ytterbium, lanthanum and europium; more preferentially, the metal(s) belonging to the group of the rare-earth metals M″ are chosen from cerium and yttrium.

Preferably, the metal belonging to the group of the rare-earth metals M″ is chosen from cerium, yttrium, ytterbium, lanthanum and europium, and mixtures thereof. More preferentially, the metal belonging to the group of the rare-earth metals is chosen from cerium and yttrium, and mixtures thereof.

Preferably, the metal belonging to the group of the rare-earth metals M″ is in the oxidation state +III.

According to the invention, the compound of a metal belonging to the group of the rare-earth metals is chosen from rare-earth metal salts and rare-earth metal complexes.

The term “rare-earth metal complex” refers to the combination of the metal M″ with one or more ligands.

In the text hereinbelow, the term “ligand” refers to an ion or a molecule bearing a group which combines, via an ionic bond and/or a coordination bond, with the metal M″. The same ligand may bear several groups which combine via an ionic bond and/or a coordination bond. A definition of rare-earth metal salts or complexes may be found in: Progress in the Science and Technology of the Rare Earths, Volume 1, edited by Leroy Eyring in 1964, published by Macmillan Company and written by F. Gaume-Mahn, page 259 et seq.

The rare-earth metal salts and complexes according to the invention are characterized in that they contain at least one metal atom M″ belonging to the group of the rare-earth metals and that said atom is in the +III oxidation state.

The metal belonging to the group of the rare-earth metals M″ may then be associated, via its electron shell, with n1 anionic groups forming an ionic bond with M″ and/or with n2 groups forming a coordination bond with M″. The groups forming a coordination bond are, for example, groups with a donor doublet, such as carbonyl or amine.

If n2=0, the compound of the metal belonging to the group of the rare-earth metals forms a salt and, in this case, the metal M″ belonging to the group of the rare-earth metals is associated with three anionic groups.

If n2>0, the compound of the metal belonging to the group of the rare-earth metals forms a complex and, in this case, the number of anionic groups n1 may range from 0 to 3.

The metal M″ belonging to the group of the rare-earth metals is associated with one or more anionic groups and/or one or more groups forming a coordination bond.

The ligands associated with the metals belonging to the group of the rare-earth metals M″ to form a corresponding rare-earth metal complex are as described below.

a) Typically, the ligand may be a monoanionic ion, which may or may not be monoatomic, such as a nitrate, a hydroxyl (OH—) or a halide (typically chloride or bromide). By way of example, the resulting rare-earth metal compound may then be M″Cl₃, M″(OH)₃ or M″(NO₃)₃, and in particular CeNO₃, YNO₃, LaNO₃, CeCl₃, YCl₃, LaCl₃, more preferentially rare-earth metal halides, notably Ce and Y halides such as CeCl₃ and YCl₃.

b) The ligand may be a dianionic or trianionic ion, such as phosphate or sulfate. By way of example, mention may be made of rare-earth metal compounds such as M″PO₄, or M″₂(SO₄)₃ and in particular CePO₄, YPO₄, LaPO₄, Ce₂(SO₄)₃, Y₂(SO₄)₃ and La₂(SO₄)₃.

c) The ligand may contain one or more groups forming a coordination bond and a function forming an ionic bond.

Thus, the ligand may be a monocarboxylate or polycarboxylate molecule, such as acetate or succinate. In this case, it is considered that the carboxylate function acts as an anionic group, by means of the hydroxyl of the carboxylic group, and acts as a group forming a coordination bond by means of the lone pair on the oxygen of the carbonyl function. Thus, the resulting rare-earth metal compound may be M″(R—(COO)ₙ)_{3/n}. In addition to bearing one or more carboxylates, the ligand may include other functions, such as hydroxyls or amines. Thus, the ligand may consist totally or partially of hydroxycarboxylic acids or aminocarboxylic acids.

As monocarboxylic or polycarboxylic compound bearing additional functions, mention may be made of tartrate, citrate, glycolate or ethylenediaminetetraacetate (EDTA) ions.

The ligand may bear a non-localized anionic charge, for instance acetylacetonate. The rare-earth metal compound will then be M″(acetylacetonate)₃ or M″(acetylacetonate)₃·7H₂O in which each acetonate bonds to the metal M″ via its two carbonyl functions, one acting as an anionic group, the other as a group bonding by coordination.

The ligand may also be of the aromatic type, such as a phenol, a cyclopentadiene (Progress in the Science and Technology of the Rare Earths, published by Leroy Eyring and written by F. Gaume-Mahn, page 296), or a pyridine.

d) The rare-earth metal compound may include one or more ligands forming a coordination bond and one or more ligands forming an ionic bond. Thus, the rare-earth metal compound may be yttrium dihydroxyacetate Y((OH)₂acetate) (Synthesis and Properties of Yttrium Hydroxyacetate Sols by S. S. Balabanov, E. M. Gavrishchuk, and D. A. Permin, Inorganic Materials, 2012, Vol. 48, No. 5, pages 500-503.)

e) The rare-earth metal compound may be a mixed salt in which one of the cations M′″ represents a cation other than a rare-earth metal cation, for instance an alkali metal or alkaline-earth metal or a cationic organic cation, notably a quaternary amine (or ammonium), for example mono/di/tri/tetra(C₁-C₄)alkylammonium, or mono/di(C₁-C₄)alkyl imidazolium, (C₁-C₄)alkylpyridinium; more particularly, the mixed salt rare-earth metal compound is Li,Ce(SO₄)₂.

The compounds belonging to the group of the rare-earth metals, which are often highly hygroscopic, may be in the form of hydrates, for instance CeCl₃·7H₂O, YCl₃·6H₂O, LaCl₃·7H₂O or Ce(acetonate)₃·xH₂O.

According to a particular embodiment of the invention, the compound(s) belonging to the group of the rare-earth metals are chosen from the salts of organic acids such as citrates, lactates, glycolates, gluconates, acetates, propionates, fumarates, oxalates, tartrates, mesylates and methosulfates, notably gluconates, hydrates thereof, and mixtures thereof.

According to a preferred embodiment, the salt(s) of a metal belonging to the group of the rare-earth metals are mineral salts.

Preferably, the mineral salt(s) of a metal belonging to the group of the rare-earth metals are chosen from halides such as chlorides, fluorides, iodides and bromides, carbonates, sulfates, phosphates, nitrates and perchlorates, hydrates thereof, and mixtures thereof.

More preferentially, the mineral salt(s) of a metal belonging to the group of the rare-earth metals are chosen from halides such as chlorides, fluorides, iodides and bromides, and nitrates, hydrates thereof, and mixtures thereof.

Even more preferentially, the mineral salt(s) of a metal belonging to the group of the rare-earth metals are chosen from chlorides and nitrates, hydrates thereof, and mixtures thereof.

According to a particularly preferred embodiment, the compound(s) belonging to the group of the rare-earth metals are chosen from Ce(NO₃)₃, Y(NO₃)₃, La(NO₃)₃, CeCl₃, YCl₃ and LaCl₃, and mixtures thereof.

According to an even more preferred embodiment, the compound(s) belonging to the group of the rare-earth metals are chosen from CeCl₃ and YCl₃, and mixtures thereof.

According to a preferred embodiment, the crosslinking agent is chosen from (poly)amino, (poly)thiolated and/or (poly)hydroxylated, (poly)carbonyl and (poly)acrylate compounds, and mixtures thereof, and preferably chosen from (poly)amino and (poly)thiolated compounds, notably with said (poly)amino being chosen from chitosans, aminoalkoxysilanes and polydimethylsiloxanes comprising primary amine groups at the end of the chain or on side chains, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane and poly dimethylsiloxanes comprising aminopropyl end groups at the end of the chain, and even more preferentially 3-aminopropyltriethoxysilane (APTES), and notably said (poly)thiolated compound being chosen from polydiorganosiloxanes bearing thiol functions, aminosilicones bearing thiol functions, alkoxysilanes bearing thiol functions, organic polythiols, natural products modified with thiol groups, amino thiols derived from dendrimers or polyethyleneimines (PEI) and silicone thiols, the (poly)thiolated compound preferably being chosen from polydimethylsiloxanes terminated with mercaptopropyl groups and dimethicone/mercaptopropyl methicone copolymers.

According to a preferred embodiment, the crosslinking agent is chosen from i) (poly)amine compounds, ii) (poly)thiolated and/or (poly)hydroxylated compounds, iii) (poly)carbonyl compounds such as terephthaldehyde, iv) (poly)acrylates such as trimethylolpropane triacrylate, v) metal salts chosen from vₐ) metal alkoxides such as titanium butoxide, v_{b}) metal (poly)(hydroxy)(C₁-C₆)alkylcarboxylates, notably of transition metals or of post-transition metals, notably aluminum, such as aluminum acetate or aluminum lactate and v_{c}) salts of metals belonging to the group of the rare-earth metals, in particular Ce or Y halides such as CeCl₃ and YCl₃, and vi) mixtures thereof, and preferably chosen from (poly)amino and (poly)thiolated compounds, with said (poly)amino notably being chosen from chitosans, aminoalkoxysilanes and polydimethylsiloxanes comprising primary amine groups at the end of the chain or on the side chains, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane and polydimethylsiloxanes containing aminopropyl end groups at the end of the chain, and even more preferentially is 3-aminopropyltriethoxysilane (APTES), and notably said (poly)thiolated compound being chosen from polydiorganosiloxanes bearing thiol functions, aminosilicones bearing thiol functions, alkoxysilanes bearing thiol functions, organic polythiols, natural products modified with thiol groups, amino thiols derived from dendrimers or polyethyleneimines (PEI) and silicone thiols, the (poly)thiolated compound preferably being chosen from polydimethylsiloxanes terminated with mercaptopropyl groups and dimethicone/mercaptopropyl methicone copolymers.

The mixtures vi) may refer to mixtures of compounds of the same type, for instance mixtures of (poly)amines i) of different structures, or mixtures of (poly)thiols ii) of different structures. The mixtures vi) may also refer to mixtures of compounds of different types, for instance mixtures consisting of one or more polyamines i) with one or more (poly)thiols ii).

According to another particular embodiment, the crosslinking agent R is a compound chosen from vₐ) a (C₁-C₆)alkoxide of a transition metal, notably of titanium, such as titanium butoxide, and v_{b}) a (poly)(hydroxy)(C₁-C₆)alkylcarboxylate of a transition metal, notably of aluminum, such as aluminum acetate, or aluminum lactate.

According to another particular embodiment, the crosslinking agent R is a compound chosen from metal alkoxides of formula (XXIIIₐ) as defined above, preferably in which the metal M is a transition metal, notably of titanium such as titanium butoxide.

According to another particular embodiment, the crosslinking agent R is a compound chosen from iii) (poly)carbonyls such as terephthaldehyde or trimethylolpropane triacrylate.

### Fatty Phase

Compositions C1, C2 or C3 according to the invention contain a fatty phase, notably a fatty phase comprising a hydrocarbon-based oil, preferably isododecane.

In a preferred embodiment, the hydrocarbon-based oil, preferably isododecane, is predominantly present in the fatty phase of a composition according to the invention, relative to all the constituents of said fatty phase.

The compositions CR and CAC may also contain a fatty phase that is suitable for use in the invention.

### Hydrocarbon-Based Oil

As stated previously, a composition C1, C2 or C3 according to the invention or CR or CAC that is suitable for use in the invention may comprise at least one hydrocarbon-based oil, in particular a volatile oil.

The term “oil” means a water-immiscible non-aqueous compound that is liquid at room temperature (20° C.) and atmospheric pressure (760 mmHg).

The term “hydrocarbon-based oil” means an oil formed essentially from, or even consisting of, carbon and hydrogen atoms, and possibly oxygen and nitrogen atoms, and not containing any silicon or fluorine atoms. It may contain alcohol, ester, ether, carboxylic acid, amine and/or amide groups.

In particular, a composition in accordance with the invention may comprise at least one hydrocarbon-based oil chosen from:
C₈-C₁₄ hydrocarbon-based oils, and notably:
branched C₈-C₁₄ alkanes, for instance C₈-C₁₄ isoalkanes (also known as isoparaffins), for instance isododecane (also known as 2,2,4,4,6-pentamethylheptane), isodecane, dodecane and, for example, the oils sold under the trade name Isopar or Permethyl, linear alkanes, for instance n-dodecane (C₁₂) and n-tetradecane (C₁₄) sold by Sasol under the respective references Parafol 12-97 and Parafol 14-97, and also mixtures thereof, the undecane-tridecane mixture, the mixtures of n-undecane (C₁₁) and of n-tridecane (C₁₃) obtained in Examples 1 and 2 of patent application WO 2008/155 059 from the company Cognis, and mixtures thereof, and
short-chain esters (containing from 3 to 8 carbon atoms in total) such as ethyl acetate, methyl acetate, propyl acetate and n-butyl acetate;
hydrocarbon-based oils of plant origin such as triglycerides constituted of fatty acid esters of glycerol, the fatty acids of which may have chain lengths ranging from C₄ to C₂₄, these chains possibly being linear or branched, and saturated or unsaturated; these oils are notably heptanoic acid or octanoic acid triglycerides, or alternatively wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or else caprylic/capric acid triglycerides, for instance those sold by the company Stéarinerie Dubois,
synthetic ethers containing from 10 to 40 carbon atoms,
linear or branched hydrocarbons of mineral or synthetic origin, in particular petroleum jelly, polydecenes, hydrogenated polyisobutene, in particular Parleam®, squalane and liquid paraffins, and mixtures thereof,
synthetic esters such as oils of formula R₁COOR₂ in which R₁ represents a linear or branched fatty acid residue including from 1 to 40 carbon atoms and R₂ represents a, notably branched, hydrocarbon-based chain containing from 1 to 40 carbon atoms, on condition that the sum of the number of carbon atoms in R₁ and R₂ is greater than or equal to 10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

Preferably, the hydrocarbon-based oil present in a composition according to the present invention is apolar, consisting only of carbon and hydrogen atoms.

Preferably, the hydrocarbon-based oil is chosen from C₈-C₁₄ hydrocarbon-based oils and mixtures thereof, and more preferentially, the hydrocarbon-based oil is chosen from isododecane, Cetiol UT, Vegelight Silk and dodecane, and even more preferentially the hydrocarbon-based oil comprises at least or is isododecane.

According to a preferred embodiment, compositions C1, C2 and/or C3 according to the invention comprise at least one hydrocarbon-based oil, preferably chosen from C₈-C₁₄ hydrocarbon-based oils and mixtures thereof, and more preferentially is at least chosen from isododecane, Cetiol UT, Vegelight Silk and dodecane, and even more preferentially is at least isododecane.

According to a preferred embodiment, compositions C1, C2 and/or C3 according to the invention comprise at least one volatile hydrocarbon-based oil.

According to a preferred embodiment, compositions C1, C2 and/or C3 according to the invention comprise at least one preferably volatile hydrocarbon-based oil and optionally one or more nonvolatile oils preferably in a volatile oils/nonvolatile oils mass ratio of greater than or equal to 2, preferably greater than or equal to 3, more preferentially greater than or equal to 4.

According to a particular embodiment, compositions C1, C2 and/or C3 according to the invention contain a fatty phase comprising isododecane, notably a fatty phase comprising a majority of isododecane relative to all the constituents of said fatty phase such as an isododecane/octyldodecanol mixture or an isododecane/isononyl isononanoate mixture.

Preferably, a composition according to the invention comprises from 15% to 98% by weight, notably from 15% to 90% by weight, preferably from 40% to 90% or even from 40% to 85% by weight, of hydrocarbon-based oil(s) relative to the total weight of the composition.

Nonvolatile Oil Different from the Hydrocarbon-Based Oil

According to a preferred embodiment, a composition C1, C2 or C3 according to the invention may also comprise at least one nonvolatile oil different from said hydrocarbon-based oil described above.

Thus, a composition according to the invention may comprise one or more fluoro or nonfluoro silicone oils or mixtures thereof. In particular, it may be an oil chosen from:
linear or branched hydrocarbons, in particular polydecenes, or hydrogenated polyisobutene, in particular Parleam;
fatty alcohols containing from 12 to 26 carbon atoms, for instance octyldodecanol, 2-butyloctanol, 2-hexyldecanol, 2-undecylpentadecanol or oleyl alcohol;
fluoro oils which are partially hydrocarbon-based and/or silicon-based;
silicone oils such as polymethylsiloxanes (PDMS) which are nonvolatile, linear or cyclic, liquid or pasty at room temperature, such as cyclomethicones, dimethicones, optionally including a phenyl group, such as phenyl trimethicones, phenyltrimethylsiloxydiphenylsiloxanes, diphenylmethyldimethyltrisiloxanes, diphenyl dimethicones, phenyl dimethicones, polymethylphenylsiloxanes;
and mixtures thereof.

The term “silicone oil” means an oil comprising at least one silicon atom and notably at least one Si—O group.

The term “fluoro oil” means an oil comprising at least one fluorine atom.

These oils may be present in a content ranging from 0.01% to 60% by weight and better still from 0.10% to 50% by weight relative to the total weight of the composition.

Preferably, the nonvolatile oil different from said hydrocarbon-based oil is chosen from nonvolatile hydrocarbon-based oils, more preferentially chosen from fatty alcohols containing from 12 to 26 carbon atoms, synthetic esters and mixtures thereof.

A composition according to the invention advantageously comprises at least one nonvolatile oil different from said hydrocarbon-based oil, preferably ranging from 0.01% to 60% by weight, and preferably from 0.1% to 50% by weight, of nonvolatile oil different from said hydrocarbon-based oil, relative to the total weight of the composition.

### Other Oils

According to a particular embodiment, a composition C1, C2 or C3 according to the invention may also comprise at least one oil different from the oils mentioned above.

Thus, a composition according to the invention may comprise hydrocarbon-based, silicone, fluoro or nonfluoro oils, or mixtures thereof, different from the oils mentioned above.

The oils may be volatile or nonvolatile.

They may be of animal, plant, mineral or synthetic origin.

The oils may optionally comprise oxygen, nitrogen, sulfur and/or phosphorus atoms, for example in the form of hydroxyl or acid radicals.

### Aqueous Phase

According to a particular embodiment, compositions C1, C2 and/or C3 according to the invention may also comprise an aqueous phase.

According to a particular embodiment, the compositions CR containing at least one crosslinking agent and/or CAC containing at least one cosmetic active agent according to the invention may also comprise an aqueous phase and/or consist of an aqueous phase.

The aqueous phase of a composition C1, C2 or C3 according to the invention or of a composition CR or CAC that is suitable for use in the invention comprises water and optionally one or more water-miscible solvents.

A water that is suitable for use in the invention may be a floral water such as cornflower water and/or a mineral water such as Vittel water, Lucas water or La Roche Posay water, a thermal spring water and/or a spring water.

In the present invention, the term “water-miscible solvent” denotes a compound that is liquid at room temperature and water-miscible (miscibility with water of greater than 50% by weight at 25° C. and atmospheric pressure).

The water-miscible solvents that may be used in the composition of the invention may also be volatile.

Among the water-miscible solvents that may be used in the composition in accordance with the invention, mention may be made notably of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

According to an embodiment variant, the aqueous phase of a composition according to the invention may comprise at least one C₂-C₃₂ polyol.

For the purposes of the present invention, the term “polyol” should be understood as meaning any organic molecule including at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those notably containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, pentane-1,2-diol, caprylyl glycol (octane-1,2-diol), butylene glycol, isoprene glycol, pentylene glycol, hexylene glycol, glycerol, polyglycerols, such as glycerol oligomers, for instance diglycerol, and polyethylene glycols, and mixtures thereof.

Preferably, a composition according to the invention is an anhydrous composition.

The term “anhydrous composition” means a composition containing less than 2% by weight of water, or even less than 0.5% of water, and is notably free of water. Where appropriate, such small amounts of water may notably be introduced by ingredients of the composition that may contain residual amounts thereof.

According to a particular form of the invention, compositions C1, C2 and/or C3 according to the invention or compositions CR or CAC may comprise at least one water-miscible solvent, preferably at least one lower monoalcohol containing from 1 to 6 carbon atoms, such as ethanol, propanol, isopropanol, butanol, isobutanol, tert-butanol, pentanol or hexanol, and/or at least one glycol containing from 2 to 8 carbon atoms, such as ethylene glycol, propylene glycol, 1,3-butylene glycol or dipropylene glycol, and/or at least one C₃-C₄ ketone and/or at least one C₂-C₄ aldehyde, more preferentially at least one lower monoalcohol containing from 1 to 6 carbon atoms as defined previously and even more preferentially at least one lower monoalcohol chosen from ethanol and n-butanol, and even more preferentially ethanol.

According to another particular form of the invention, compositions C1, C2 and/or C3 according to the invention or compositions CR or CAC are anhydrous (i.e. they contain less than 5% by weight, in particular less than 2% by weight or even less than 0.5% by weight of water) and comprise at least one lower monoalcohol containing from 1 to 6 carbon atoms such as ethanol, propanol, isopropanol, butanol, isobutanol, tert-butanol, pentanol or hexanol and/or at least one glycol containing from 2 to 8 carbon atoms such as ethylene glycol, propylene glycol, 1,3-butylene glycol or dipropylene glycol, and/or at least one C₃-C₄ ketone and/or at least one C₂-C₄ aldehyde, preferably at least one lower monoalcohol containing from 1 to 6 carbon atoms as defined previously and more preferentially at least one lower monoalcohol chosen from ethanol and n-butanol, and even more preferentially ethanol.

### Cosmetic Active Agent

A composition according to the invention, notably C1 or C2 or a composition that is suitable for use in the invention such as CR, may comprise one or more cosmetic active agent(s) termed “CAA”, chosen from:
a) coloring agents chosen from pigments, direct dyes, and mixtures thereof,
b) active agents for caring for keratin materials, preferably the skin,
c) UV-screening agents, and
d) mixtures thereof.

The cosmetic active agent(s) CAA optionally present in composition C1 may be introduced with the copolymer CP and/or with a different composition CAC.

The cosmetic active agent(s) CAA optionally present in a composition C2 may be introduced with the crosslinking agent R and/or a composition CR, and/or with a composition C1 and/or with a composition CAC.

A composition C3 according to the invention or a composition CAC that is suitable for use in the invention comprises one or more cosmetic active agent(s) CAA chosen from a) coloring agents chosen from pigments, direct dyes, and mixtures thereof, b) active agents for caring for keratin materials, preferably the skin, c) UV-screening agents, and d) mixtures thereof.

The cosmetic active agents CAA present in a composition C3 may be introduced therein in different ways depending on the protocol adopted for preparing this composition.

They may thus be introduced with at least one of the ingredients of composition C3, for example with the (co)polymer in composition C1 and/or with the crosslinking agent R or with a different composition CR containing at least this crosslinking agent and/or with a composition CAC.

In the variant in which composition C3 is obtained from a composition C1, they may be present in this composition C1 and/or be implemented with a composition CAC. In the variant in which composition C3 is obtained from a composition C2, such CAA(s) may be present in this composition C2 and/or be used with a composition CAC.

Needless to say, in each of these variants, one or more identical or different CAAs may be present in the compositions considered to prepare C3.

Similarly, the same cosmetic active agent CAA may be present in a composition C1 and/or in a composition C2 and/or in a composition CR and/or in a separate composition CAC, it being understood that, when several different cosmetic active agents are used, they may be introduced, totally or partially, either into C1 and/or into C2 and/or into CR and/or into a separate composition CAC.

If several cosmetic active agents are present in a composition C3, they may be present simultaneously in a composition C1 and/or in a composition C2 and/or in a composition CR and/or in a separate composition CAC.

According to another variant, if several cosmetic active agents are present in a composition C3, each of them may be present separately in a composition C1 and/or in a composition C2 and/or in a composition CR and/or in a separate composition CAC.

According to a particular embodiment, the at least one cosmetic agent is chosen from dyestuffs, preferably chosen from pigments, direct dyes and mixtures thereof.

Needless to say, a person skilled in the art will take care to select this or these optional cosmetic active agent(s) CAA, and/or the amount thereof, such that the advantageous properties of the corresponding composition according to the invention are not, or are not substantially, adversely affected by the envisaged addition.

### Dyestuffs

A composition according to the invention such as C1 or C2, or a composition which is suitable for use in the invention such as CR, may also comprise at least one particulate or non-particulate, water-soluble or water-insoluble dyestuff, preferably in a proportion of at least 0.01% by weight relative to the total weight of the composition considered.

A composition C3 according to the invention, or a composition which is suitable for use in the invention such as CAC, also comprises a particulate or non-particulate, water-soluble or water-insoluble dyestuff, preferably in a proportion of at least 0.01% by weight relative to the total weight of the composition considered.

For obvious reasons, this amount is liable to vary significantly with regard to the intensity of the desired color effect and of the color intensity afforded by the dyestuffs under consideration, and its adjustment clearly falls within the competence of a person skilled in the art.

Preferably, a composition C3 according to the invention comprises at least one dyestuff chosen from pigments, direct dyes and mixtures thereof.

Preferably, composition CAC which is suitable for use in the invention comprises at least one dyestuff chosen from pigments, direct dyes and mixtures thereof, preferably at least one pigment.

When a composition C1, C2 or CR according to the invention comprises at least one cosmetic active agent CAA, said cosmetic active agent preferably comprises at least one dyestuff chosen from pigments, direct dyes and mixtures thereof and more preferentially at least one pigment.

Preferably, a composition C3 according to the invention comprises at least one pigment.

### Pigments

For the purposes of the invention, the term “pigment” means any compound that is capable of imparting color to keratin materials. These compounds have a solubility in water at 25° C. and at atmospheric pressure (760 mmHg) of less than 0.05% by weight, and preferably less than 0.01% by weight.

A composition C1 or C2 according to the invention or a composition CR that is suitable for use in the invention may comprise at least one cosmetic active agent CAA, said cosmetic active agent preferably comprising one or more pigments.

A composition C3 according to the invention or a composition CAC that is suitable for use in the invention comprises at least one cosmetic active agent CAA, said cosmetic active agent preferably comprising one or more pigments.

As pigments that are suitable for use in the invention, mention may notably be made of the organic and/or mineral pigments known in the art, notably those described in Kirk-Othmer's Encyclopedia of Chemical Technology and in Ullmann's Encyclopedia of Industrial Chemistry.

These pigments may be synthetic or natural.

These pigments may be in pigment powder or paste form. They may be coated or uncoated.

These pigments may be chosen, for example, from mineral pigments, organic pigments, lakes, pigments with special effects such as nacres or glitter flakes, and mixtures thereof.

A pigment that is suitable for use in the invention may be chosen from mineral pigments.

The term “mineral pigment” means any pigment that satisfies the definition in Ullmann's encyclopedia in the chapter on inorganic pigments.

Among the mineral pigments that are useful in the present invention, mention may be made of manganese violet, ultramarine blue, chromium hydrate, ferric blue and titanium, zirconium or cerium oxides or dioxides, and also of zinc, iron or chromium oxides.

They may also be pigments having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30. They may also be pigments having a structure that may be, for example, of silica microsphere type containing iron oxide. An example of a pigment having this structure is the product sold by the company Miyoshi under the reference PC Ball PC-LL-100 P, this pigment being constituted of silica microspheres containing yellow iron oxide.

Advantageously, the pigments may be iron oxides and/or titanium dioxides.

A pigment that is suitable for use in the invention may be chosen from organic pigments.

The term “organic pigment” means any pigment that satisfies the definition in Ullmann's encyclopedia in the chapter on organic pigments.

Among the organic pigments that are useful in the present invention, mention may be made of nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

In particular, the white or colored organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:
Cosmenyl Yellow 10G: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may notably be composed of particles including a mineral core, at least one binder for attaching the organic pigments to the core, and at least one organic pigment which at least partially covers the core.

The organic pigment may also be a lake. The term “lake” refers to dyes adsorbed onto insoluble particles, the assembly thus obtained remaining insoluble during use.

The mineral substrates onto which the dyes are adsorbed are, for example, alumina, silica, calcium sodium borosilicate or calcium aluminum borosilicate and aluminum.

Among the dyes adsorbed on organic substrates, mention may be made of carminic acid. Mention may also be made of the dyes known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 4 (CI 15 510), D & C Red 33 (CI 17 200), D & C Yellow 5 (CI 19 140), D & C Yellow 6 (CI 15 985) D & C Green (CI 61 570), D & C Yellow 10 (CI 77 002), D & C Green 3 (CI 42 053), D & C Blue 1 (CI 42 090), FDC Red 4, D & C Red 6, D & C Red 22, D & C Red 28, D & C Red 30, D & C Orange 4, D & C Yellow 8, D & C Green 5, D & C Red 17, D & C Green 6, D & C Yellow 11, D & C Purple 2, Sudan red, carotenes (0-carotene, lycopene), xanthophylls (capsanthin, capsorubin, lutein), palm oil, Sudan brown, quinoline yellow, annatto, curcumin, betanin (beet), carmine, copper chlorophyllin, methylene blue, anthocyanins (enocianine, black carrot, hibiscus, elderberry), caramel, riboflavin, beet juice and caramel.

An example of a lake that may be mentioned is the product known under the following name: D&C Red 7 (CI 15 850:1).

The pigment may also be a pigment with special effects. The term “pigments with special effects” means pigments that generally create a colored appearance (characterized by a certain shade, a certain vivacity and a certain level of luminance) that is non-uniform and that changes as a function of the conditions of observation (light, temperature, angles of observation, etc.). They thereby differ from colored pigments, which afford a standard uniform opaque, semi-transparent or transparent shade.

Several types of pigments with special effects exist: those with a low refractive index, such as fluorescent or photochromic pigments, and those with a higher refractive index, such as nacres, interference pigments or glitter flakes.

The term “nacres” should be understood as meaning iridescent or non-iridescent colored particles of any shape, notably produced by certain molluscs in their shell, or alternatively synthesized, which have a color effect via optical interference.

Examples of pigments with special effects that may be mentioned include nacreous pigments such as mica coated with titanium or with bismuth oxychloride, colored nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. They may also be mica particles, at the surface of which are superposed at least two successive layers of metal oxides and/or of organic dyestuffs.

Nacreous pigments that may be mentioned include the nacres Cellini sold by BASF (mica-TiO₂-lake), Prestige sold by Eckart (mica-TiO₂), Prestige Bronze sold by Eckart (mica-Fe₂O₃) and Colorona sold by Merck (mica-TiO₂—Fe₂O₃).

Mention may also be made of the gold-colored nacres sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by the company Merck under the name Bronze fine (17384) (Colorona) and Bronze (17353) (Colorona) and by the company BASF under the name Super bronze (Cloisonne); the orange nacres sold notably by the company BASF under the name Orange 363C (Cloisonne) and Orange MCR 101 (Cosmica) and by the company Merck under the name Passion orange (Colorona) and Matte orange (17449) (Microna); the brown nacres sold notably by the company BASF under the name Nu-antique copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan opale G005 (Gemtone); the black nacres with a gold tint sold notably by the company BASF under the name Nu antique bronze 240 AB (Timica), the blue nacres sold notably by the company Merck under the name Matte blue (17433) (Microna), the white nacres with a silvery tint sold notably by the company Merck under the name Xirona Silver, and the golden-green pink-orange nacres sold notably by the company Merck under the name Indian summer (Xirona), and mixtures thereof.

Still as examples of nacres, mention may also be made of particles including a borosilicate substrate coated with titanium oxide.

Particles comprising a glass substrate coated with titanium oxide are notably sold under the name Metashine MC1080RY by the company Toyal.

Finally, examples of nacres that may also be mentioned include polyethylene terephthalate glitter flakes, notably those sold by the company Meadowbrook Inventions under the name Silver 1P 0.004×0.004 (silver glitter flakes). It is also possible to envisage multilayer pigments based on synthetic substrates, such as alumina, silica, calcium sodium borosilicate, calcium aluminum borosilicate and aluminum.

Advantageously, the nacres in accordance with the invention are micas covered with titanium dioxide or with iron oxide, and also bismuth oxychloride.

The pigments with special effects may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. brighter points that contrast with their environment, making them appear to sparkle.

The reflective particles may be selected so as not to significantly alter the coloring effect generated by the coloring agents with which they are combined, and more particularly so as to optimize this effect in terms of color rendition. They may more particularly have a yellow, pink, red, bronze, orange, brown, gold and/or coppery color or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or mineral materials.

More particularly, the substrate of the reflective particles may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Illustrations of these particles that may be mentioned include aluminum particles, such as those sold under the names Starbrite 1200 EAC® by the company Siberline and Metalure® by the company Eckart and glass particles coated with a metallic layer, notably those described in JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with a nickel/chromium/molybdenum alloy are sold under the names Crystal Star GF 550 and GF 2525 by this same company.

Use may also be made of particles comprising a metal substrate, such as silver, aluminum, iron, chromium, nickel, molybdenum, gold, copper, zinc, tin, magnesium, steel, bronze or titanium, said substrate being coated with at least one layer of at least one metal oxide, such as titanium oxide, aluminum oxide, iron oxide, cerium oxide, chromium oxide, silicon oxides and mixtures thereof.

Examples that may be mentioned include aluminum powder, bronze powder or copper powder coated with SiO₂ sold under the name Visionaire by the company Eckart.

Mention may also be made of interference pigments which are not attached to a substrate, such as liquid crystals (Helicones HC from Wacker) or interference holographic glitter flakes (Geometric Pigments or Spectra f/x from Spectratek). Special effect pigments also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colors, and also particular optical effects such as metallic effects or interference effects.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C₈ to C₂₀ fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments surface-treated beforehand that are useful in the context of the invention are pigments which have been totally or partially subjected to a surface treatment of chemical, electronic, electrochemical, mechanochemical or mechanical nature with an organic agent, such as those described notably in Cosmetics and Toiletries, February 1990, Vol. 105, pages 53-64, before being dispersed in the composition in accordance with the invention. These organic agents may be chosen from waxes, for example carnauba wax and beeswax; fatty acids, fatty alcohols and derivatives thereof, such as stearic acid, hydroxystearic acid, stearyl alcohol, hydroxystearyl alcohol, lauric acid and derivatives thereof; anionic surfactants; sodium, potassium, magnesium, iron, titanium, zinc or aluminum salts of fatty acids, for example aluminum stearate or laurate, aluminum dimyristate and the aluminum salt of hydrogenated tallow glutamate; metal alkoxides; polyethylene; (meth)acrylic polymers, for example polymethyl methacrylates; polymers and copolymers containing acrylate units; alkanolamines; silicone compounds, for example silicones, notably polydimethylsiloxanes; fluoro organic compounds, for example perfluoroalkyl ethers, perfluoroalkyl phosphates, hexafluoropropylene polyoxides, perfluoropolyethers; fluorosilicone compounds such as perfluoroalkylsilanes; amino acids; N-acylamino acids or salts thereof; lecithin, isopropyl triisostearyl titanate, isostearyl sebacate; and mixtures thereof.

The term “alkyl” mentioned in the compounds cited previously notably denotes an alkyl group containing from 1 to 30 carbon atoms and preferably containing from 5 to 16 carbon atoms.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface treatments of the pigments are chosen from the following treatments:
a PEG-silicone treatment, for example the AQ surface treatment sold by LCW;
a methicone treatment, for example the SI surface treatment sold by LCW;
a dimethicone treatment, for example the Covasil 3.05 surface treatment sold by LCW;
a dimethicone/trimethyl siloxysilicate treatment, for example the Covasil 4.05 surface treatment sold by LCW;
a magnesium myristate treatment, for example the MM surface treatment sold by LCW;
an aluminum dimyristate treatment, for example the MI surface treatment sold by Miyoshi;
a perfluoropolymethyl isopropyl ether treatment, for example the FHC surface treatment sold by LCW;
an isostearyl sebacate treatment, for example the HS surface treatment sold by Miyoshi;
a perfluoroalkyl phosphate treatment, for example the PF surface treatment sold by Daito;
an acrylate/dimethicone copolymer and perfluoroalkyl phosphate treatment, for example the FSA surface treatment sold by Daito;
a polymethylhydrogenosiloxane/perfluoroalkyl phosphate treatment, for example the FS01 surface treatment sold by Daito;
an acrylate/dimethicone copolymer treatment, for example the ASC surface treatment sold by Daito;
an isopropyl titanium triisostearate treatment, for example the ITT surface treatment sold by Daito;
an acrylate copolymer treatment, for example the APD surface treatment sold by Daito;
a perfluoroalkyl phosphate/isopropyl titanium triisostearate treatment, for example the PF+ITT surface treatment sold by Daito.

According to a particular embodiment of the invention, the dispersant is present with organic or mineral pigments in submicron-sized particulate form in the dye composition.

The term “submicron” refers to pigments having a particle size that has been micronized by a micronization method and having a mean particle size of less than a micrometre (μm); in particular, the mean particle size is from 0.1 to 0.9 μm and preferably from 0.2 to 0.6 μm.

According to one embodiment, the dispersant and the pigment(s) are present in a dispersant/pigment mass ratio from 1:4 to 4:1, particularly from 1.5/3.5 to 3.5:1 or better still from 1.75:3 to 3:1.

The dispersant(s) may thus have a silicone backbone, such as silicone polyether and dispersants of amino silicone type other than the alkoxysilanes described previously. Among the suitable dispersants that may be mentioned are:
amino silicones, i.e. silicones comprising one or more amino groups such as those sold under the names and references: BYK LPX 21879 by BYK, GP-4, GP-6, GP-344, GP-851, GP-965, GP-967 and GP-988-1, sold by the company Genesee Polymers,
silicone acrylates such as those sold under the names and references Tego® RC 902, Tego® RC 922, Tego® RC 1041 and Tego® RC 1043 by the company Evonik,
polydimethylsiloxane (PDMS) silicones bearing carboxyl groups such as those sold under the names and references X-22162 and X-22370 by the company Shin-Etsu, epoxy silicones such as those sold under the names and references GP-29, GP-32, GP-502, GP-504, GP-514, GP-607, GP-682, and GP-695 by the company Genesee Polymers, or Tego® RC 1401, Tego® RC 1403 and Tego® RC 1412 by the company Evonik.

According to a particular embodiment, the dispersant(s) are of amino silicone type other than the alkoxysilanes described previously and are cationic.

Preferably, the pigment(s) are chosen from mineral, mixed mineral-organic or organic pigments.

According to a particular variant of the invention, the pigment(s) according to the invention are organic pigments, preferentially organic pigments surface-treated with an organic agent chosen from silicone compounds.

According to another embodiment of the invention, the pigment(s) according to the invention are mineral pigments.

A composition C1 or C2 according to the invention or a composition CR that is suitable for use in the invention may comprise at least one cosmetic active agent CAA, said cosmetic active agent preferably comprising one or more direct dyes.

A composition C3 according to the invention or a composition CAC that is suitable for use in the invention comprises at least one cosmetic active agent CAA, said cosmetic active agent preferably comprising one or more direct dyes.

The term “direct dye” means natural and/or synthetic dyes, other than oxidation dyes. These are dyes that will spread superficially on the fiber. They may be ionic or nonionic, preferably cationic or nonionic.

Among the direct dyes that are suitable for use in the invention, mention may be made of azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (A) and (B) below and the azo cationic dyes of formulae (C) and (D) below:

Het⁺-N(Ra)—N═C(Rb)—Ar, Q− (B)

Het⁺-N═N—Ar, Q− (C)

Ar⁺—N═N—Ar″, Q− (D)

in which:
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₅)alkylammonium, such as trimethylammonium;
Ar represents an aryl group, notably phenyl, which is optionally substituted, preferentially with one or more electron-donating groups such as optionally substituted (C₁-C₈)alkyl, optionally substituted (C₁-C₈)alkoxy, (di)(C₁-C₈)(alkyl)amino optionally substituted on the alkyl group(s) with a hydroxyl group, aryl(C₁-C₈)alkylamino, and optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group; or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (A) to (D) as defined previously, more particularly, the cationic direct dyes bearing an endocyclic cationic charge described in patent applications WO 95/15144, WO 95/01772 and EP 714 954, preferentially the following direct dyes of formulae (E) and (F):

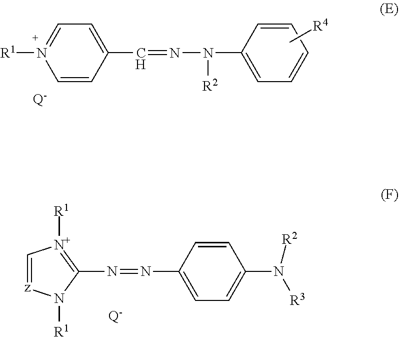

in which:
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
Z represents a CH group or a nitrogen atom, preferentially CH;
Q⁻ is an anionic counterion as defined previously, in particular a halide, such as chloride, or an alkyl sulfate, such as methyl sulfate or mesyl.

In particular, the dyes of formulae (E) and (F) are chosen from Basic Red 51, Basic Yellow 87 and Basic Orange 31 or derivatives thereof with Q− being an anionic counterion as defined previously, particularly a halide such as chloride, or an alkyl sulfate such as methyl sulfate or mesyl.

The direct dyes may be chosen from anionic direct dyes. The anionic direct dyes of the invention are dyes commonly referred to as “acid” direct dyes owing to their affinity for alkaline substances.

The term “anionic direct dye” means any direct dye including in its structure at least one CO₂R′ or SO₃R′ substituent with R′ denoting a hydrogen atom or a cation originating from a metal or an amine, or an ammonium ion.

The anionic direct dyes may be chosen from direct nitro acid dyes, azo acid dyes, azine acid dyes, triarylmethane acid dyes, indoamine acid dyes, anthraquinone acid dyes, indigoid dyes and natural acid dyes.

As direct anionic dyes that are suitable for use in the invention, mention may be made of the dyes of formulae (G), (G′), (H), (H′), (J), (J′), (K), (K′), (L), (M), (N) and (0) below:
a) the anionic azo dyes of formulae (G) and (G′) below:

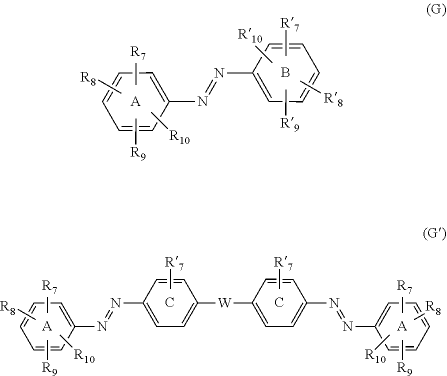

in which R₇, R₈, R₉, R₁₀, R′₇, R′₈, R′₉ and R′₁₀, which may be identical or different, represent a hydrogen atom or a group chosen from:
alkyl;
alkoxy, alkylthio;
hydroxyl, mercapto;
nitro, nitroso;
R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
R″—S(O)₂—, with R″ representing a hydrogen atom or an alkyl, aryl, (di)(alkyl)amino or aryl(alkyl)amino group; preferentially a phenylamino or phenyl group;
R′″—S(O)₂—X′— with R′″ representing an optionally substituted alkyl or aryl group, X′ being as defined previously;
(di)(alkyl)amino;
aryl(alkyl)amino optionally substituted with one or more groups chosen from i) nitro; ii) nitroso; iii) (O)₂S(O⁻)—, M⁺ and iv) alkoxy with M as defined previously;
optionally substituted heteroaryl; preferentially a benzothiazolyl group;
cycloalkyl, notably cyclohexyl;
Ar—N═N— with Ar representing an optionally substituted aryl group; preferentially a phenyl optionally substituted with one or more alkyl, (O)₂S(O⁻)—, M⁺ or phenylamino groups;
or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from nitro; nitroso; (O)₂S(O⁻)—, M⁺; hydroxyl; mercapto; (di)(alkyl)amino; R^{o}—C(X)—X′—; R^{o}—X′—C(X)—; R^{o}—X′—C(X)—X″—;
optionally substituted Ar—N═N— and aryl(alkyl)amino; with M⁺, R^{o}, X, X′, X″ and Ar as defined previously;
and in which:
W represents a sigma bond a, an oxygen or sulfur atom, or a divalent radical —NR— with R as defined previously, or methylene —C(Ra)(Rb)— with Ra and Rb, which may be identical or different, representing a hydrogen atom or an aryl group, or alternatively Ra and Rb form, together with the carbon atom that bears them, a spiro cycloalkyl; preferentially, W represents a sulfur atom or Ra and Rb together form a cyclohexyl;
it being understood that formulae (G) and (G′) comprise at least one sulfonate radical (O)₂S(O⁻)—, M⁺ or one carboxylate radical (O)CO⁻—, M⁺ on one of the rings A, A′, B, B′ or C; preferentially sodium sulfonate.

As examples of dyes of formula (G), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3; Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2; Food Yellow 3 or Sunset Yellow; and as examples of dyes of formula (G′), mention may be made of: Acid Red 111, Acid Red 134, Acid Yellow 38;
b) the pyrazolone anionic azo dyes of formulae (H) and (H′) below:

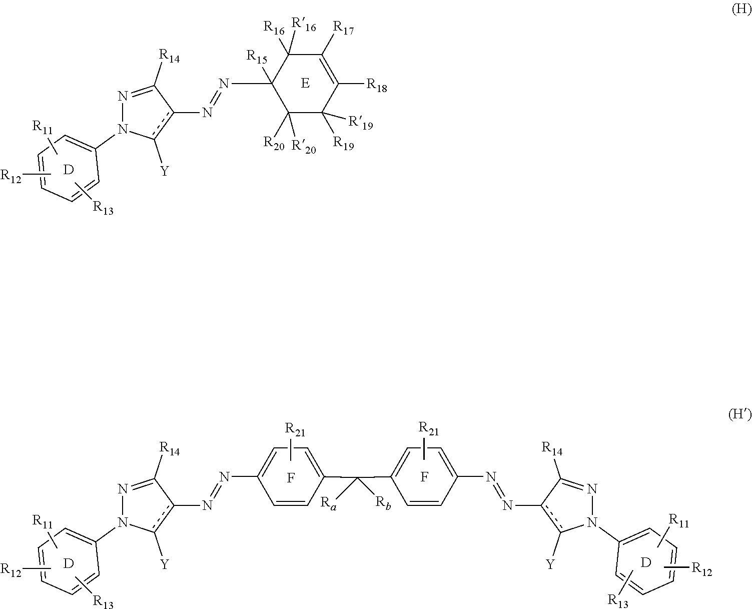

in which:
   R₁₁, R₁₂ and R₁₃, which may be identical or different, represent a hydrogen or halogen atom, an alkyl group or —(O)₂S(O⁻), M⁺ with M⁺ as defined previously;
   R₁₄ represents a hydrogen atom, an alkyl group or a group —C(O)O⁻, M⁺ with M⁺ as defined previously;
   R₁₅ represents a hydrogen atom;
   R₁₆ represents an oxo group, in which case R′₁₆ is absent, or alternatively R₁₅ with R₁₆ together form a double bond;
   R₁₇ and R₁₈, which may be identical or different, represent a hydrogen atom, or a group chosen from:
   (O)₂S(O⁻)—, M⁺ with M⁺ as defined previously;
   Ar—O—S(O)₂— with Ar representing an optionally substituted aryl group, preferentially a phenyl optionally substituted with one or more alkyl groups;
   R₁₉ and R₂₀ together form either a double bond, or a benzo group D′, which is optionally substituted;
   R′₁₆, R′₁₉ and R′₂₀, which may be identical or different, represent a hydrogen atom or an alkyl or hydroxyl group;
   R₂₁ represents a hydrogen atom or an alkyl or alkoxy group;
   Rₐ and R_{b}, which may be identical or different, are as defined previously; preferentially, Rₐ represents a hydrogen atom and R_{b} represents an aryl group;
   Y represents either a hydroxyl group or an oxo group; [symbol]
represents a single bond when Y is an oxo group; and represents a double bond when Y represents a hydroxyl group;
it being understood that formulae (H) and (H′) comprise at least one sulfonate radical (O)₂S(O⁻)—, M⁺ or one carboxylate radical —C(O)O⁻, M⁺ on one of the rings D or E;
preferentially sodium sulfonate.

As examples of dyes of formula (H), mention may be made of: Acid Red 195, Acid Yellow 23, Acid Yellow 27, Acid Yellow 76, and as an example of a dye of formula (H′), mention may be made of: Acid Yellow 17;
c) the anthraquinone dyes of formulae (J) and (J′) below:

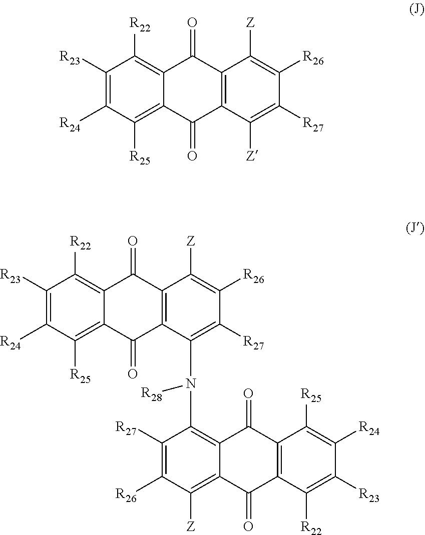

in which R²², R²³, R²⁴, R²⁵, R²⁶ and R²⁷, which may be identical or different, represent a hydrogen or halogen atom, or a group chosen from:
   alkyl;
   hydroxyl, mercapto;
   alkoxy, alkylthio;
   optionally substituted aryloxy or arylthio, preferentially substituted with one or more groups chosen from alkyl and (O)₂S(O⁻)—, M⁺ with M⁺ as defined previously;
   aryl(alkyl)amino optionally substituted with one or more groups chosen from alkyl and (O)₂S(O⁻)—, M⁺ with M⁺ as defined previously;
   (di)(alkyl)amino;
   (di)(hydroxyalkyl)amino;
   (O)₂S(O⁻)—, M⁺ with M⁺ as defined previously;
and in which:
Z′ represents a hydrogen atom or a group NR₂₈R₂₉ with R₂₈ and R₂₉, which may be identical or different, representing a hydrogen atom or a group chosen from:
   alkyl;
   polyhydroxyalkyl such as hydroxyethyl;
   aryl optionally substituted with one or more groups, particularly i) alkyl such as methyl, n-dodecyl, n-butyl; ii) (O)₂S(O⁻)—, M⁺ with M⁺ as defined previously; iii) R^{o}—C(X)—X′—, R^{o}—X′—C(X)—, R^{o}—X′—C(X)—X″— with R^{o}, X, X′ and X″ as defined previously, preferentially R^{o} represents an alkyl group;
   cycloalkyl; notably cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously;
it being understood that formulae (J) and (J′) comprise at least one sulfonate radical (O)₂S(O⁻)—, M⁺ or one carboxylate radical —C(O)O⁻, M⁺; preferentially sodium sulfonate.

As examples of dyes of formula (J), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2; and, as an example of a dye of formula (J′), mention may be made of: Acid Black 48;
d) the nitro dyes of formulae (K) and (K′) below:

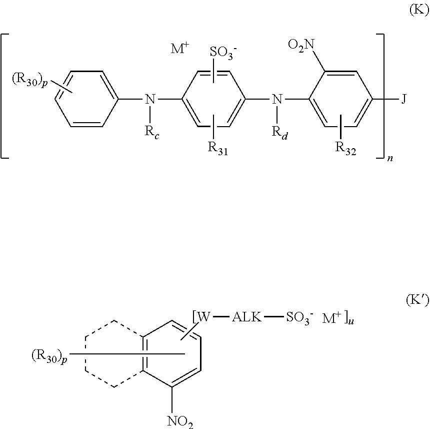

in which R₃₀, R₃₁ and R₃₂, which may be identical or different, represent a hydrogen or halogen atom, or a group chosen from:
   alkyl;
   alkoxy optionally substituted with one or more hydroxyl groups, alkylthio optionally substituted with one or more hydroxyl groups;
   hydroxyl, mercapto;
   nitro, nitroso;
   polyhaloalkyl;
   R^{o}—C(X)—X′—, R^{o}—X′—C(X)—, R^{o}—X′—C(X)—X″— with R^{o}, X, X′ and X″ as defined previously;
   (O)₂S(O⁻)—, M⁺ with M⁺ as defined previously;
   (O)CO⁻—, M⁺ with M⁺ as defined previously;
   (di)(alkyl)amino;
   (di)(hydroxyalkyl)amino;
   heterocycloalkyl such as piperidino, piperazino or morpholino; in particular, R₃₀, R₃₁ and R₃₂ represent a hydrogen atom;
and in which:
   Rc and Rd, which may be identical or different, represent a hydrogen atom or an alkyl group;
   W is as defined previously; W particularly represents an —NH— group;
   ALK represents a linear or branched divalent C₁-C₆ alkylene group; in particular, ALK represents a —CH₂—CH₂— group;
   n is 1 or 2;
   p represents an integer inclusively between 1 and 5;
   q represents an integer inclusively between 1 and 4;
   u is 0 or 1;
   when n is 1, J represents a nitro or nitroso group; particularly nitro;
   when n is 2, J represents an oxygen or sulfur atom, or a divalent radical —S(O)ₘ— with m representing an integer 1 or 2; preferentially, J represents an —SO₂— radical;
   M′ represents a hydrogen atom or a cationic counterion;

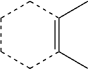

which may be present or absent, represents a benzo group optionally substituted with one or more groups R₃₀ as defined previously;
it being understood that formulae (K) and (K′) comprise at least one sulfonate radical (O)₂S(O⁻)—, M⁺ or one carboxylate radical —C(O)O—, M⁺; preferentially sodium sulfonate.

As examples of dyes of formula (K), mention may be made of: Acid Brown 13 and Acid Orange 3; as examples of dyes of formula (K′), mention may be made of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-O-hydroxyethylamino-3-nitrobenzenesulfonic acid; EXT D&C Yellow 7;
e) the triarylmethane dyes of formula (L) below:

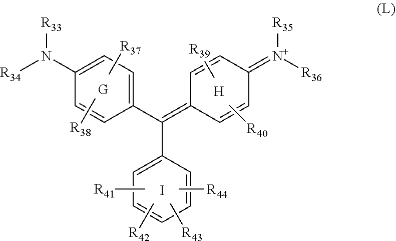

in which:
   R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined previously;
   R₃₇, R₃₈, R₃₉, R₄₀, R₄₁, R₄₂, R₄₃ and R₄₄, which may be identical or different, represent a hydrogen atom or a group chosen from:
   alkyl;
   alkoxy, alkylthio;
   (di)(alkyl)amino;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
   or alternatively two contiguous groups R₄₁ with R₄₂ or R₄₂ with R₄₃ or R₄₃ with R₄₄ together form a fused benzo group: I′; with I′ optionally substituted with one or more groups chosen from nitro; nitroso; (O)₂S(O⁻)—, M⁺; hydroxyl; mercapto; (di)(alkyl)amino; R^{o}—C(X)—X′—; R^{o}—X′—C(X)— and R^{o}—X′—C(X)—X″—; with M⁺, R^{o}, X, X′ and X″ as defined previously;
in particular, R₃₇ to R₄₀ represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with an (O)₂S(O⁻)— group;
it being understood that at least one of the rings G, H, I or I′ comprises at least one sulfonate radical (O)₂S(O⁻)— or one carboxylate radical —C(O)O—; preferentially sulfonate.

As examples of dyes of formula (L), mention may be made of: Acid Blue 1, Acid Blue 3, Acid Blue 7, Acid Blue 9, Acid Violet 49, Acid Green 3, Acid Green 5, Acid Green 50.
f) the xanthene-based dyes of formula (M):

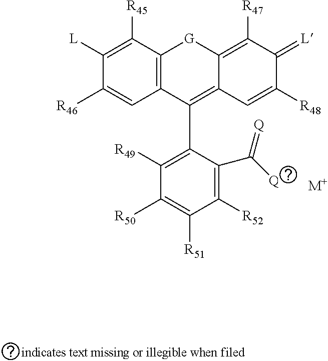

in which R₄₅, R₄₆, R₄₇ and R₄₈, which may be identical or different, represent a hydrogen atom or a halogen atom and R₄₉, R₅₀, R₅₁ and R₅₂, which may be identical or different, represent a hydrogen or halogen atom or a group chosen from:
   alkyl;
   alkoxy, alkylthio;
   hydroxyl, mercapto;
   nitro, nitroso;
   (O)₂S(O⁻)—, M⁺ with M⁺ representing a hydrogen atom or a cationic counterion;
   (O)CO⁻—, M⁺ with M⁺ as defined previously;
particularly, R₅₃, R₅₄, Rss and R₄₈ represent a hydrogen or halogen atom;
and in which:
   G represents an oxygen or sulfur atom or a group NRe with Re as defined previously; particularly, G represents an oxygen atom;
   L represents an alkoxide O⁻, M⁺; a thioalkoxide S⁻, M⁺ or a group NRf, with Rf representing a hydrogen atom or an alkyl group, and M⁺ as defined above; M⁺ is particularly sodium or potassium;
   L′ represents an oxygen or sulfur atom or an ammonium group: N⁺RfRg, with Rf and Rg, which may be identical or different, representing a hydrogen atom or an optionally substituted alkyl or aryl group; L′ particularly represents an oxygen atom or a phenylamino group optionally substituted with one or more alkyl or (O)ₘS(O⁻)—, M⁺ groups with m and M⁺ as defined previously;
   Q and Q′, which may be identical or different, represent an oxygen or sulfur atom; particularly, Q and Q′ represent an oxygen atom;
   M⁺ is as defined previously.

As examples of dyes of formula (M), mention may be made of: Acid Yellow 73, Acid Red 51, Acid Red 52, Acid Red 87, Acid Red 92, Acid Red 95, Acid Violet 9.
g) the indole-based dyes of formula (N):

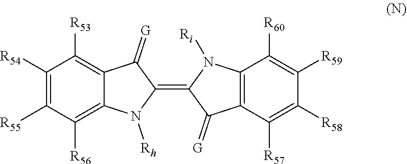

in which R₅₃, R₅₄, R₅₅, R₅₆, R₅₇, R₅₈, R₅₉ and R₆₀, which may be identical or different, represent a hydrogen atom or a group chosen from:
   alkyl;
   alkoxy, alkylthio;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
and in which:
   G represents an oxygen or sulfur atom or a group NRe with Re as defined previously; in particular, G represents an oxygen atom;
   Ri and Rh, which may be identical or different, represent a hydrogen atom or an alkyl group;
it being understood that formula (N) comprises at least one sulfonate radical (O)₂S(O⁻)—, M⁺ or one carboxylate radical —C(O)O⁻, M⁺; preferentially sodium sulfonate.

As examples of dyes of formula (N), mention may be made of: Acid Blue 74.
h) the quinoline-based dyes of formula (O) below:

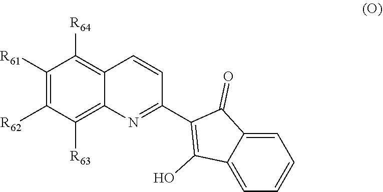

in which:
   R₆₁ represents a hydrogen or halogen atom or an alkyl group;
   R₆₂, R₆₃ and R₆₄, which may be identical or different, represent a hydrogen atom or a group (O)₂S(O⁻)—, M⁺ with M⁺ representing a hydrogen atom or a cationic counterion;
or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
it being understood that formula (XVII) comprises at least one sulfonate radical (O)₂S(O⁻)—, M⁺, preferentially sodium sulfonate.

As examples of dyes of formula (O), mention may be made of: Acid Yellow 2, Acid Yellow 3 and Acid Yellow 5.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

Preferably, the direct dyes are chosen from anionic direct dyes.

The dyestuffs, preferably the pigments, may be present in concentrations ranging from 0.01% to 30% by weight, preferably from 0.02% to 20% by weight and more particularly from 0.05% to 15% relative to the total weight of the composition containing them.

The direct dye(s) may be present in concentrations ranging from 0.0010% to 10% by weight relative to the total weight of the composition, preferably from 0.005% to 5% by weight relative to the total weight of the composition containing them.

Preferably, the cosmetic active agent(s) CAA, in particular the dyestuff(s) and more particularly the pigment(s), are introduced into the compositions C1 under consideration to prepare the compositions C3.

### Care Active Agents

A composition C1, C2, or a composition CR that is suitable for use in the invention, may comprise, as cosmetic active agent, at least one care active agent, and preferably in an amount of at least 0.01% by weight relative to the total weight of the composition under consideration.

According to a particular form of the invention, a composition C3 according to the invention, or a composition CAC that is suitable for use in the invention, comprises, as cosmetic active agent, at least one care active agent, and preferably in an amount of at least 0.010% by weight relative to the total weight of the composition under consideration.

In particular, the care active agent may be at least one hydrophilic active agent and/or one lipophilic active agent, and preferably a hydrophilic care active agent.

The term “hydrophilic active agent” means a water-soluble or water-dispersible active agent that is capable of forming hydrogen bonds.

The cosmetic care active agent(s) may notably be chosen from:
vitamins and derivatives thereof, notably esters thereof, in particular tocopherol (vitamin E) and esters thereof (for instance tocopheryl acetate), ascorbic acid (vitamin C) and derivatives thereof;
humectants, in particular urea, hydroxyureas, glycerol, polyglycerols, glyceryl glucoside, diglyceryl glucoside, polyglyceryl glucosides and xylityl glucoside, and in particular glycerol;
C-glycoside compounds;
antioxidant compounds;
antiaging active agents, in particular hyaluronic acid compounds, and notably sodium hyaluronate, retinol and derivatives thereof, salicylic acid compounds and in particular 5-n-octanoylsalicylic acid (capryloylsalicylic acid), caffeine, adenosine, C-β-D-xylopyranoside-2-hydroxypropane and the sodium salt of (3-hydroxy-2-pentylcyclopentyl)acetic acid;
skincare agents chosen from allantoin, panthenol and protein hydrolyzates;
venotonic agents, in particular polyphenols, and notably escin, ruscus, diosmin, hesperidin, resveratrol, and
mixtures thereof.

Preferably, the at least one care active agent comprises at least one skincare active agent.

Preferably, the at least one care active agent comprises at least one moisturizer (also called a humectant) and preferably comprises at least glycerol.

According to a particular form of the invention, a composition C1, C2 according to the invention, or a composition CR that is suitable for use in the invention, may comprise, as cosmetic active agent, at least one care active agent, and preferably at least one moisturizer (also called a humectant).

According to a particular form of the invention, a composition C3 according to the invention, or a composition CAC that is suitable for use in the invention, comprises, as cosmetic active agent, at least one care active agent, and preferably at least one moisturizer (also called a humectant).

Preferably, the care active agent is a moisturizer, and in particular is glycerine (glycerol).

The care active agent(s) may in particular be present, in the composition under consideration, in a content ranging from 0.010% to 30% by weight, relative to the weight of the composition, and preferably from 0.02% to 25% by weight.

Preferably, the care active agent(s), in particular glycerol, are introduced into the compositions C1.

### UV-Screening Agents

A composition C1 or C2 according to the invention, or a composition CR that is suitable for use in the invention, may comprise, as cosmetic active agent, at least one UV-screening agent.

A composition C3 according to the invention, or a composition CAC that is suitable for use in the invention, comprises, as cosmetic active agent, at least one UV-screening agent.

The UV-screening agent is a UV-screening agent normally used in cosmetics. It may be chosen from the positive list contained in Annex VI of (EC) Regulation No. 1223/2009, which specifies the list of UV-screening agents permitted in cosmetics.

The UV-screening agents that are suitable for use in the invention may be of varied nature. They may be lipophilic, hydrophilic or insoluble organic agents.

The term “lipophilic UV-screening agent” means any cosmetic or dermatological screening agent that can be fully dissolved in molecular form in a liquid fatty phase or that can be dissolved in colloidal form (for example in micellar form) in a liquid fatty phase.

The term “hydrophilic UV-screening agent” means any cosmetic or dermatological screening agent that can be fully dissolved in molecular form in a liquid aqueous phase or that can be dissolved in colloidal form (for example in micellar form) in a liquid aqueous phase.

The term “insoluble UV-screening agent” means any cosmetic or dermatological screening agent which is not defined either as a lipophilic UV-screening agent or as a hydrophilic UV-screening agent, and which is in the form of particles in aqueous phase or liquid fatty phase.

The UV-screening agents of the composition according to the invention may afford UVA and/or UVB photoprotection.

According to a preferred embodiment, the compositions according to the invention, which are preferably cosmetic compositions, may comprise at least one organic and/or mineral UV-screening agent (for screening out the UV radiation of sunlight).

In particular, a composition C1 or C2 according to the invention, or a composition CR that is suitable for use in the invention, may comprise, as cosmetic active agent, at least one UV-screening agent chosen from hydrophilic organic UV-screening agents, lipophilic organic UV-screening agents, insoluble organic UV-screening agents, mineral screening agents, and mixtures thereof.

In particular, a composition C3 according to the invention, or a composition CAC that is suitable for use in the invention, may comprise, as cosmetic active agent, at least one UV-screening agent chosen from hydrophilic organic UV-screening agents, lipophilic organic UV-screening agents, insoluble organic UV-screening agents, mineral screening agents, and mixtures thereof.

In particular, these compositions may comprise one or more UV-screening agents chosen from bis-resorcinyl triazine derivatives, dibenzoylmethane derivatives, benzylidenecamphor derivatives, and mixtures thereof.

The organic UV-screening agents may also be chosen from anthranilic derivatives; cinnamic derivatives; salicylic derivatives; benzophenone derivatives; phenylbenzotriazole derivatives; benzalmalonate derivatives, notably those mentioned in patent U.S. Pat. No. 5,624,663; phenylbenzimidazole derivatives; imidazolines; 4,4-diarylbutadiene derivatives; bis-benzazolyl derivatives, as described in patents EP 6 693 23 and U.S. Pat. No. 2,463,264; p-aminobenzoic acid (PABA) derivatives; methylenebis(hydroxyphenylbenzotriazole) derivatives, as described in patent applications U.S. Pat. Nos. 5,237,071, 5,166,355, GB 2 303 549, DE 197 26, 184 and EP 893 119; benzoxazole derivatives, such as those described in patent applications EP 0 832 642, EP 1 027 883, EP 1 300 137 and DE 101 62 844; screening polymers and screening silicones such as those notably described in patent application WO 93/04665; α-alkylstyrene-based dimers such as those described in patent application DE 198 55 649; 4,4-diarylbutadienes such as those described in patent applications EP 0 967 200, DE 197 46 654, DE 197 55 649, EP 1 008 586, EP 1 133 980 and EP 133 981; other merocyanine derivatives such as those described in patent applications WO 04/006878, WO 05/058269 and WO 06/032741, and mixtures thereof.

According to a particular embodiment, the concentration of the organic UV-screening agents in the compositions according to the invention ranges from 1% to 50%, preferably from 1% to 40% by weight, and better still, for example, ranges from 5% to 35% by weight, relative to the total weight of the composition.

The compositions according to the invention may also comprise mineral UV-screening agents, which are generally pigments. The pigments may or may not be coated.

The coated pigments are pigments that have undergone one or more surface treatments of chemical, electronic, mechanochemical and/or mechanical nature with compounds as described, for example, in Cosmetics & Toiletries, February 1990, Vol. 105, pages 53-64, such as amino acids, beeswax, fatty acids, fatty alcohols, anionic surfactants, lecithins, sodium, potassium, zinc, iron or aluminum salts of fatty acids, metal alkoxides (titanium or aluminum alkoxides), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and consist essentially of a repetition of main units in which the silicon atoms are linked together via oxygen atoms (siloxane bond), optionally substituted hydrocarbon-based radicals being directly attached via a carbon atom to said silicon atoms.

The term “silicones” also encompasses the silanes required for their preparation, in particular alkylsilanes.

The silicones used for coating the pigments that are suitable for the present invention are preferably chosen from the group containing alkylsilanes, polydialkylsiloxanes and polyalkylhydrogenosiloxanes. Even more preferentially, the silicones are chosen from the group containing octyltrimethylsilane, polydimethylsiloxanes and polymethylhydrogenosiloxanes.

Needless to say, before being treated with silicones, the metal oxide pigments may have been treated with other surface agents, in particular with cerium oxide, alumina, silica, aluminum compounds or silicon compounds, or mixtures thereof.

Thus, the mineral UV-screening agents may be chosen from coated or uncoated pigments, and in particular from coated titanium oxide pigments, silicone-treated titanium oxides, uncoated titanium oxide pigments, uncoated zinc oxide pigments, coated zinc oxide pigments, uncoated cerium oxide pigments, uncoated iron oxide pigments, coated iron oxide pigments, and mixtures thereof.

Mention may also be made of mixtures of metal oxides, in particular of titanium dioxide and of cerium dioxide, including the equal-weight mixture of titanium dioxide and cerium dioxide coated with silica, sold by the company Ikeda under the name Sunveil A, and also the mixture of titanium dioxide and zinc dioxide coated with alumina, silica and silicone, such as the product M 261 sold by the company Kemira, or coated with alumina, silica and glycerol, such as the product M 211 sold by the company Kemira.

The pigments may be introduced into the compositions according to the invention in their native form or in the form of a pigmentary paste, i.e. as a mixture with a dispersant, as described, for example, in GB-A-2 206 339.

According to a particular embodiment, the compositions according to the invention are free of mineral UV-screening agents.

According to a particular embodiment, the amount of the mineral UV-screening agent(s) present in the compositions according to the invention may range from 0.01% to 20% by weight relative to the total weight of the composition. It ranges, for example, from 1% to 15% by weight, relative to the total weight of the composition.

According to a particular embodiment, the composition according to the invention also comprises one or more organic UV-screening agents and one or more mineral UV-screening agents.

According to a particular embodiment, the compositions according to the invention comprise a combination of UV-screening agents as described in patent FR 2 977 490, patent application WO 2013/004777 or patent application US 2014/0134120.

Preferably, the water-soluble UV-screening agent(s), are introduced into a composition according to the invention and in particular into a composition C3, by being mixed with composition C1 if under consideration to prepare a composition C3 and/or with composition CR containing at least one crosslinking agent.

Preferably, the liposoluble UV-screening agent(s) are introduced into a composition according to the invention and in particular into a composition C3, via a different composition CAC.

According to another preferred mode, the mineral or insoluble UV-screening agent(s) are present in composition C1.

### Fillers

A composition according to the invention may also comprise one or more fillers, notably in a content ranging from 0.01% to 30% by weight, relative to the total weight of the composition, preferably ranging from 0.01% to 20% by weight relative to its total weight.

The term “fillers” means colorless or white, mineral or synthetic particles of any shape, which are insoluble in the medium of the composition, irrespective of the temperature at which the composition is manufactured.

These fillers may notably serve to modify the rheology or texture of the composition.

As illustrations of these fillers, mention may be made of talc, mica, silica, kaolin, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), acrylic acid copolymer, silicone resin microbeads (for example Tospearls® from Toshiba), polyorganosiloxane elastomer particles, precipitated calcium carbonate, magnesium carbonate, magnesium hydrogen carbonate, hydroxyapatite, barium sulfate, aluminum oxides, polyurethane powders, composite fillers, hollow silica microspheres, and glass or ceramic microcapsules. Use may also be made of particles that are in the form of hollow sphere portions, as described in patent applications JP-2003 128 788 and JP-2000 191 789.

### Other Active Agents

According to a preferred embodiment, a composition C1 or C2 or C3 according to the invention or a composition CR or CAC that is suitable for use in the invention comprises at least one additional cosmetic active agent.

A composition according to the invention may also contain ingredients commonly used in cosmetics, such as thickeners, gelling agents, trace elements, softeners, sequestrants, fragrances, basifying or acidifying agents, dispersants, preserving agents, surfactants, hair loss counteractants, antidandruff agents, propellants, polar additives, film-forming polymers different from the copolymers CP of the invention, or mixtures thereof.

Needless to say, a person skilled in the art will take care to select this or these optional additional compound(s), and/or the amount thereof, such that the advantageous properties of a composition according to the invention are not, or are not substantially, adversely affected by the envisioned addition.

As nonlimiting illustrations of a composition C1, mention may be made of compositions comprising at least:
a copolymer obtained by copolymerizing at least isobornyl acrylate, 2-ethylhexyl acrylate and acetoacetoxyethyl methacrylate monomers in isododecane and preferably at least one pigment,
a copolymer obtained by copolymerizing at least isobornyl acrylate and acetoacetoxyethyl methacrylate monomers in isododecane, at least one nonvolatile oil different from isododecane, preferably 2-octyl-1-decanol, and preferably at least one pigment,
a copolymer obtained by copolymerizing at least isobornyl acrylate, 2-ethylhexyl acrylate, PDMS methacrylate and acetoacetoxyethyl methacrylate monomers in isododecane, and preferably at least one pigment,
a copolymer obtained by copolymerizing at least isobornyl acrylate and acetoacetoxyethyl methacrylate monomers in isododecane, preferably a nonvolatile oil other than isododecane, notably chosen from 2-octyl-1-decanol and isononyl isononanoate and preferably at least one pigment, or
a copolymer obtained by copolymerizing at least isobornyl acrylate, butyl acrylate and acetoacetoxyethyl methacrylate monomers in isododecane and preferably at least one pigment.

As nonlimiting illustrations of compositions C2 or C3, mention may be made of compositions comprising at least:
a copolymer obtained by copolymerizing at least isobornyl acrylate, 2-ethylhexyl acrylate and acetoacetoxyethyl methacrylate monomers in isododecane, a crosslinking agent chosen from 3-aminopropyltriethoxysilane (APTES), poly(D-glucosamine), [4-6% (mercaptopropyl)methylsiloxane]dimethylsiloxane copolymer and bis(3-aminopropyl)-terminated poly(dimethylsiloxane) (PDMS-diNH₂) and preferably at least one pigment,
a copolymer obtained by copolymerizing at least isobornyl acrylate and acetoacetoxyethyl methacrylate monomers in isododecane, a crosslinking agent chosen from poly(D-glucosamine), bis(3-aminopropyl)-terminated poly(dimethylsiloxane) (PDMS-diNH₂), bis-cetearyl amodimethicone, optionally at least one nonvolatile oil different from isododecane, preferably chosen from isononyl isononanoate and 2-octyl-1-decanol, and preferably at least one pigment,
a copolymer obtained by copolymerizing at least isobornyl acrylate, 2-ethylhexyl acrylate, PDMS methacrylate and acetoacetoxyethyl methacrylate monomers in isododecane, a crosslinking agent chosen from poly(D-glucosamine) and bis-cetearyl amodimethicone, and preferably at least one pigment, or
a copolymer obtained by copolymerizing at least isobornyl acrylate, butyl acrylate and acetoacetoxyethyl methacrylate monomers in isododecane, bis-cetearyl amodimethicone and preferably at least one pigment.

### Composition

A composition C1, C2 or C3 in accordance with the invention may be in the form of a makeup product, in particular a colored makeup product, for the skin, in particular a foundation, optionally having care properties, a blusher, a face powder, an eyeshadow, a concealer, an eyeliner; a lip makeup product such as a lipstick, optionally having care properties, a lip gloss or lip pencils; a makeup product for the integuments such as the nails or eyelashes, in particular in the form of a cake mascara, or for the eyebrows and the hair, or a product for the temporary tattooing of bodily skin.

According to a particular embodiment, a composition C1, C2 or C3 according to the invention is in the form of:
either a colored product for the lips;
or a skincare product, which may be colored, in particular a cream or a fluid having moisturizing and/or filling and/or tensor properties; or
or a haircare product, in particular a hair dyeing product or a styling product notably free of dyestuff, such as a lacquer, or a “styling” product of the mousse or gel type.

In particular, a composition according to the invention is not in the form of a patch.

### Process for Applying the Compositions According to the Invention

Compositions C1, C2 and C3 according to the invention may be applied directly as such to the target keratin material or may even be formed directly on the surface of this keratin material.

According to the invention, three application methods known as the “one-gesture application mode”, the “two-gesture application mode” and the “three-gesture application mode” are thus distinguished.

The term “one-gesture application method” means the direct application to the target keratin material of a single composition in accordance with the invention, namely a composition C1 according to the invention or a composition C2 or C3 according to the invention comprising at least one fatty phase, at least one copolymer CP and at least one crosslinking agent R as described above.

The term “two-gesture application method” means the successive application, to the target keratin material, of two different compositions, at least one of which is according to the invention. Thus, this two-gesture application method covers the sequential application to a target keratin material of α) a composition C1 optionally containing at least one cosmetic active agent CAA, and β) a second composition chosen from a composition CR containing at least one crosslinking agent R, and optionally at least one cosmetic active agent CAA, as defined previously, CAA preferably being a dyestuff.

According to this application method, step α) is preferably followed by step β).

The term “three-gesture application method” means the sequential application to a target keratin material of α) a composition CAC comprising one or more cosmetic active agent(s), β) a composition C1 and γ) a composition CR comprising at least one crosslinking agent R, as defined previously.

According to this application method, step γ) preferably constitutes the last step of said process and more preferentially step α) is followed by step β), which is itself followed by step γ).

In the two- or three-gesture application methods, composition C1 is conventionally referred to as the “base coat” and the composition which is superimposed on it, generally the composition CR, is referred to as the “top coat”.

These three application methods are detailed and illustrated below.

### “One-Gesture” Application Method

According to one embodiment, a single composition C1 is applied to the keratin materials.

According to another embodiment, a single composition C2 or C3 is applied to the keratin materials.

According to this embodiment, a composition C1 is mixed with at least one crosslinking agent R or a composition CR and optionally with at least one cosmetic active agent CAA or a different composition CAC before application to the keratin material.

Advantageously, the system remains fluid long enough to allow application to the keratin materials, for instance the skin or the hair.

According to a particular embodiment, composition C2 is prepared just before application to the keratin materials, for example by mixing a composition C1 and a crosslinking agent R or by mixing a composition C1 and a composition CR containing at least one crosslinking agent R.

According to a particular embodiment, the resulting composition C2 or C3 is applied to dry keratin materials.

According to a particular embodiment, the keratin materials may be dried after application of the resulting composition C2 or C3.

After application of the composition, a persistent, non-tacky deposit is advantageously obtained. The deposit obtained is also resistant to food oils, water and shampoo washing.

### “Two-Gesture” Application Method

According to a particular embodiment, a composition according to the invention may be implemented according to a “two-gesture” application method.

According to such an embodiment, i) a composition C1 according to the invention and ii) a crosslinking agent R or a composition CR comprising at least one crosslinking agent R, CR and R as described above, are applied sequentially to the keratin materials.

Preferably, step ii) is subsequent to step i).

According to one particular embodiment, the compositions are applied to dry keratin materials.

According to another particular embodiment, the keratin materials may be dried after application of the compositions, in particular after application of each composition.

After application of the two compositions, a persistent, non-tacky deposit is advantageously obtained. The deposit obtained is also resistant to food oils, water and shampoo washing.

### “Three-Gesture” Application Method

According to a particular embodiment, a composition according to the invention may be implemented according to a “three-gesture” application method.

According to this embodiment, i) a composition CAC containing at least one cosmetic active agent CAA, notably at least one dyestuff, preferably at least one pigment, ii) a composition C1 comprising at least one copolymer CP as defined previously and iii) a crosslinking agent R or a composition CR comprising at least one crosslinking agent R, CR and R as described above, and different from the composition CAC, are applied sequentially to the keratin materials.

Preferably, step iii) is subsequent to step ii).

According to such an embodiment, preferably, a composition CAC comprising at least one cosmetic active agent CAA, notably at least one dyestuff, and preferably at least one pigment, is applied to the keratin materials.

Successively, a composition C1 as described above, comprising at least one copolymer CP and at least one fatty phase, preferably isododecane, as described above, is applied to the composition CAC.

Successively, a composition CR comprising at least one crosslinking agent R, as described above and different from said composition CAC, is applied to the compositions CAC and C1.

According to a particular embodiment, the compositions CAC, C1 and CR are prepared just before application to the keratin materials.

According to one particular embodiment, the compositions are applied to dry keratin materials.

According to a particular embodiment, the keratin materials may be dried after application of the compositions, in particular after application of each composition.

Thus, the compositions C1, C2, C3 and optionally the compositions CAC and CR may be applied to dry or wet keratin fibers, which may or may not have been rinsed, and also to all types of light or dark, natural or dyed, permed, bleached or straightened fibers.

According to a particular embodiment of the invention, the fibers are washed before application of one of the compositions C1, C2, C3 and optionally of a composition CAC and/or CR. The application to the fibers may be performed via any conventional means, in particular using a comb, a fine brush, a coarse brush, a sponge or with the fingers.

According to one embodiment, the compositions C1, C2, C3 and optionally the compositions CAC and CR are applied to the keratin fibers with a leave-on time of between 1 minute and 10 hours, in particular between 1 minute and 1 hour.

After application of the composition C1, C2 or C3 and optionally of one or more compositions CAC and/or CR, the keratin fibers may optionally be rinsed.

The keratin fibers are then optionally dried or left to dry, for example at a temperature of greater than or equal to 30° C.

According to a particular embodiment, the fibers may be dried at a temperature of greater than or equal to 40° C. According to a particular embodiment, the fibers may be dried at a temperature of greater than 40° C. and less than 100° C.

Preferably, if the fibers are dried, they are dried, in addition to a supply of heat, with a flow of air. During drying, a mechanical action may be exerted on the locks, such as combing, brushing or running the fingers through. This operation may similarly be performed once the fibers have dried, naturally or otherwise.

The drying step may be performed with a drying device such as a hood, a hairdryer, a climazone, etc.

When the drying step is performed with a hood or a hairdryer, the drying temperature is between 40 and 110° C. and preferably between 50 and 90° C.

After the drying step, a shaping step may be performed, for example with a straightening iron; the temperature for the shaping step is between 110 and 220° C., preferably between 140 and 200° C.

After the drying step, a final rinse and/or shampoo wash may optionally be performed.

This type of application advantageously makes it possible to obtain persistent, non-tacky deposits that are resistant to greasy substances such as food oils or sebum, and also to water and shampoo washing.

In addition, a three-gesture application advantageously makes it possible to improve the cosmeticity and to provide sheen and volume to the keratin materials.

More particularly, according to one of its aspects, the present invention relates to a process, notably a cosmetic process, for keratin materials, in particular for caring for and/or making up the skin, lips, eyelashes and/or eyebrows and/or for caring for, styling and/or dyeing keratin fibers, and preferably the hair, comprising a step of applying to said keratin materials a composition C1 according to the invention, said composition containing at least one copolymer CP as defined previously, at least one fatty phase i) and optionally at least one cosmetic active agent, termed “CAA”, chosen from a) coloring agents chosen from pigments, direct dyes and mixtures thereof, b) active agents for caring for keratin materials, preferably the skin, c) UV-screening agents and d) mixtures thereof.

According to another of its aspects, the present invention relates to a process, notably a cosmetic process, for keratin materials, in particular for caring for and/or making up the skin, lips, eyelashes and/or eyebrows and/or for caring for, styling and/or dyeing keratin fibers and preferably the hair, comprising a step of applying to said keratin materials a composition C2 or C3 according to the invention, said compositions containing at least one copolymer CP, at least one crosslinking agent R and at least one fatty phase as defined previously and optionally at least one cosmetic active agent CAA as defined previously, in particular at least one dyestuff and notably at least one pigment.

According to another aspect, the present invention relates to a process, notably a cosmetic process, for keratin materials, in particular for caring for and/or making up the skin, lips, eyelashes and/or eyebrows and/or for caring for, styling and/or dyeing keratin fibers and preferably the hair, comprising at least the steps consisting in:
(a) placing all or part of the surface of said keratin materials in contact with a composition C1 according to the invention, which is preferably free of crosslinking agent and optionally containing at least one cosmetic active agent CAA as defined previously; and
(b) placing all or part of said surface in contact with a composition CR different from said composition C1 and comprising at least one crosslinking agent, in particular a crosslinking agent R as defined above, which is preferably free of copolymer CP as defined previously; with it being possible for steps (a) and (b) to be performed simultaneously or sequentially, and it being understood that said process may optionally use at least one cosmetic active agent, termed “CAA”, contained in composition C1 and/or in composition CR and/or optionally in a composition which is different from C1 and from CR, termed “CAC”, said CAA being chosen from a) coloring agents chosen from pigments, direct dyes and mixtures thereof, b) active agents for caring for keratin materials, preferably the skin, c) UV-screening agents, and d) mixtures thereof.

### Cosmetic Treatment Process

According to another of its aspects, the present invention relates to a process for making up the skin, notably the lips, the eyelashes or the eyebrows, comprising a step of applying to the skin, or said lips or eyelashes or eyebrows, a composition C3, notably containing at least one dyestuff notably as defined previously and more particularly at least one pigment.

According to another of its aspects, the present invention relates to a process for making up the skin, notably the lips, the eyelashes or the eyebrows, comprising at least the sequential application:
of a composition C1 as described above,
of a crosslinking agent R or of a composition CR containing at least one crosslinking agent R,
   it being understood that said composition C1 or composition CR contains at least one dyestuff as defined previously, preferably at least one pigment. Preferably, composition C1 is applied before the crosslinking agent R or composition CR containing at least one crosslinking agent R.

According to another of its aspects, the present invention relates to a process for making up the skin, notably the lips, eyelashes or eyebrows, comprising at least the three successive steps consisting in sequentially applying i) a composition CAC containing at least one dyestuff CAA, ii) a composition C1, iii) a crosslinking agent R or a composition CR containing at least one crosslinking agent R, it being understood that said process uses at least one cosmetic active agent CAA denoting a dyestuff, notably a pigment.

Preferably, composition C1 is applied before the crosslinking agent R or composition CR containing at least one crosslinking agent R.

Preferably, said makeup process comprises at least the steps consisting in:
applying to the skin, notably the lips, the eyelashes or the eyebrows, a composition CAC containing at least one dyestuff CAA, notably at least one pigment and optionally at least one care active agent as defined previously,
applying, in contact with said composition CAC applied to the skin, the eyelashes or the eyebrows, a composition C1,
applying, in contact with said composition C1, applied in contact with said composition CAC, a crosslinking agent R or a composition CR comprising at least one crosslinking agent.

According to another of its aspects, the present invention relates to a process for the care, notably the nontherapeutic and cosmetic care, of the skin, notably of the face or the lips, comprising a step of applying to the skin at least one composition C1, C2 or C3 according to the invention.

According to another of its aspects, the present invention relates to a process for the care, notably the nontherapeutic and cosmetic care, of the skin, notably of the face or the lips, comprising a step of applying to the skin a composition C3, notably containing at least one moisturizing cosmetic active agent, in particular glycerol, and optionally at least one dyestuff as defined previously.

According to another of its aspects, the present invention relates to a process for the care, notably the nontherapeutic and cosmetic care, of the skin, notably of the face or the lips, comprising the sequential application:
of a composition C1 as described above, and
of a crosslinking agent R or of a composition CR containing at least one crosslinking agent R,
   it being understood that said composition C1 and/or said composition CR contains at least one moisturizing cosmetic active agent such as glycerol and optionally at least one dyestuff. Preferably, composition C1 is applied before the crosslinking agent R or composition CR containing at least one crosslinking agent R.

According to yet another of its aspects, the present invention relates to a process for the care, notably the nontherapeutic and cosmetic care, of the skin, notably of the face or the lips, comprising at least the sequential application:
of a composition CAC containing at least one cosmetic active agent CAA,
of a composition C1,
of a crosslinking agent R or of a composition CR comprising at least one crosslinking agent R,
   it being understood that said process uses at least one cosmetic active agent CAA denoting a care active agent, notably a moisturizing active agent, in particular glycerol, and optionally at least one dyestuff as defined previously.

Preferably, composition C1 is applied before the crosslinking agent R or composition CR containing at least one crosslinking agent R.

Preferably, said process comprises at least the three successive steps consisting in:
applying to said skin a composition CAC containing at least one cosmetic active agent CAA, notably at least one care active agent, preferably at least one moisturizing active agent such as glycerol, and optionally at least one dyestuff as defined previously,
applying, in contact with said composition CAC applied to the skin, a composition C1,
applying, in contact with said composition C1, applied in contact with said composition CAC, a crosslinking agent R or a composition CR comprising at least one crosslinking agent R.

According to another of its aspects, the present invention relates to a process for dyeing keratin fibers, preferably human keratin fibers such as the hair, comprising a step of applying to said keratin fibers a composition C1, C2 or C3 according to the invention, said composition C1, C2 or C3 containing at least one dyestuff, notably as defined previously.

According to another of its aspects, the present invention relates to a process for dyeing keratin fibers, preferably human keratin fibers such as the hair, comprising a step of applying to said keratin fibers a composition C3 according to the invention, said composition C3 containing at least one coloring agent, notably as defined previously.

According to another of its aspects, the present invention relates to a process for dyeing keratin fibers, preferably human keratin fibers such as the hair, comprising at least the sequential application:
of a composition C1 as described above, and
of a crosslinking agent R or of a composition CR containing at least one crosslinking agent R,
   it being understood that said composition C1 or composition CR contains at least one dyestuff as defined previously, preferably at least one pigment.

Preferably, composition C1 is applied before the crosslinking agent R or composition CR containing at least one crosslinking agent R.

According to another of its aspects, the present invention relates to a process for dyeing keratin fibers, preferably human keratin fibers such as the hair, comprising at least the three successive steps consisting in sequentially applying i) a composition CAC containing at least one dyestuff CAA, ii) a composition C1, iii) a crosslinking agent R or a composition CR containing at least one crosslinking agent R, it being understood that said process uses at least one cosmetic active agent CAA denoting a dyestuff, notably a pigment.

Preferably, composition C1 is applied before the crosslinking agent R or composition CR containing at least one crosslinking agent R.

Preferably, said dyeing process comprises at least the steps consisting in:
applying to said keratin fiber a composition CAC containing at least one dyestuff CAA, notably at least one pigment and optionally at least one care active agent as defined previously,
applying, in contact with said composition CAC applied to the keratin fiber, a composition C1,
applying, in contact with said composition C1, applied in contact with said composition CAC, a crosslinking agent R or a composition CR comprising at least one crosslinking agent.

According to another of its aspects, the present invention relates to a process for styling keratin fibers, notably human keratin fibers and in particular the hair, comprising a step of applying to the keratin fibers a composition C1, C2 or C3 as defined previously according to the invention.

According to another of its aspects, the present invention relates to a process for styling keratin fibers, preferably human keratin fibers such as the hair, comprising a step of applying to said keratin fibers a composition C3 according to the invention, said composition C3 optionally containing at least one cosmetic active agent CAA, notably a dyestuff as defined previously.

According to another of its aspects, the present invention relates to a process for styling keratin fibers, preferably human keratin fibers such as the hair, comprising at least the sequential application:
of a composition C1 as described previously, and
of a crosslinking agent R or of a composition CR containing at least one crosslinking agent R,
   it being understood that said composition C1 or composition CR optionally contains at least one cosmetic active agent CAA, notably a dyestuff as defined previously, preferably at least one pigment. Preferably, composition C1 is applied before the crosslinking agent R or composition CR containing at least one crosslinking agent R.

According to another of its aspects, the present invention relates to a process for styling keratin fibers, preferably human keratin fibers such as the hair, comprising at least the three successive steps consisting in sequentially applying i) a composition CAC containing at least one cosmetic active agent CAA, ii) a composition C1, iii) a crosslinking agent R or a composition CR containing at least one crosslinking agent R.

Preferably, composition C1 is applied before the crosslinking agent R or composition CR containing at least one crosslinking agent R.

In particular, said styling process comprises at least the steps consisting in:
applying to said keratin fiber a composition CAC containing at least one cosmetic active agent CAA, notably at least one dyestuff, notably at least one pigment and optionally at least one care active agent as defined previously,
applying, in contact with said composition CAC applied to the keratin fiber, a composition C1, and
applying, in contact with said composition C1, applied in contact with said composition CAC, a crosslinking agent R or a composition CR comprising at least one crosslinking agent R.

### Kit

According to yet another of its aspects, the present invention is also directed toward a kit, notably a cosmetic kit, for keratin materials, in particular for caring for and/or making up the skin and/or the lips, eyelashes or eyebrows, and/or for caring for, styling and/or dyeing keratin fibers and preferably the hair, comprising:
a first compartment containing at least one fatty phase as defined above and at least one copolymer CP, optionally at least one cosmetic active agent CAA as defined above, and preferably free of crosslinking agent R;
a second compartment separate from the first and containing at least one crosslinking agent R, in particular as defined above, preferably free of copolymer CP; and
optionally, a third compartment separate from the first and second compartments and containing at least one cosmetic active agent CAA, which may be identical to or different from that/those possibly contained in the first and second compartments and as defined above;
   it being understood that the first and/or second compartments may also contain one or more identical or different cosmetic active agents CAA and that said cosmetic active agent CAA is chosen from a) coloring agents chosen from pigments, direct dyes and mixtures thereof, b) active agents for caring for keratin materials, preferably the skin, in particular moisturizers such as glycerol, c) UV-screening agents, and d) mixtures thereof.

Throughout the description, including the claims, the term “including a” should be understood as being synonymous with “including at least one”, unless otherwise specified.

The expressions “between . . . and . . . ”, “comprises from . . . to . . . ”, “formed from . . . to . . . ” and “ranging from . . . to . . . ” should be understood as being inclusive of the limits, unless otherwise specified.

The invention is illustrated in greater detail by the examples presented below. Unless otherwise indicated, the amounts indicated are expressed as mass percentages.

### EXAMPLES

### Methods and Measurements

### 1. Styling Application: Styling Evaluation Protocol

The styling evaluation protocol is detailed below:

A 1 g lock of natural hair containing 90% white hair strands is wrapped around a brush.

2 g of a solution containing the polymer at 10% in isododecane or, respectively, in a 50/50 isododecane/ethanol mixture are sprayed onto the lock. The lock is weighed before and after application. About 0.5 g of the polymer-containing solution is effectively deposited on the lock.

The lock is measured immediately after application and 24 hours later, after storage at room temperature.

A comparison is made with a lock onto which only isododecane or, respectively, only a 50/50 isododecane/ethanol mixture has been sprayed.

### 2. Hair Dyeing Application

The hair dyeing evaluation protocol is detailed below:

The evaluations are conducted according to three different protocols: in 1, 2 or 3 step(s), each on locks of natural hair containing 90% white hair strands.

### a) One-Step Protocol

The composition is prepared just before application.

It is applied to dry hair in a bath ratio of 0.5 g of formula per gram of hair.

The lock is dried with a hairdryer.

The evaluations in terms of resistance to shampoo washing are performed 24 hours after applying the composition.

The resistance to shampoo washing may then be evaluated visually and/or by measuring the colorimetric data of each lock with a Minolta CM-3610d type spectrophotometer.

### b) Two-Step Protocol

A first composition, termed the “base coat”, is applied to dry hair in a bath ratio of 0.5 g of composition per gram of hair.

The lock is dried with a hairdryer.

Three hours after the application of the “base coat”, a second composition, termed the “top coat”, is applied in a bath ratio of 0.5 g of composition per gram of hair.

The lock is again dried with a hairdryer.

The lock may also be straightened with a straightening iron at a temperature of 180° C., by applying ten passes evenly along the entire length of the lock.

The evaluations in terms of resistance to shampoo washing are performed 24 hours after applying the “top coat”.

The resistance to shampoo washing may then be evaluated visually and/or by measuring the colorimetric data of each lock with a Minolta CM-3610d type spectrophotometer.

### c) Three-Step Protocol

A composition comprising a pigment in a cosmetic medium such as water or isododecane is applied to dry hair.

The lock is dried with a hairdryer.

Immediately after drying, a composition, termed the “base coat”, is applied in a bath ratio of 0.5 g of composition per gram of hair.

The lock is again dried with a hairdryer.

Three hours after the application of the “base coat”, a composition, termed the “top coat”, is applied in a bath ratio of 0.5 g of composition per gram of hair.

The lock is once again dried with a hairdryer.

The evaluations in terms of resistance to shampoo washing are performed 24 hours after applying the “top coat”.

The resistance to shampoo washing may then be evaluated visually and/or by measuring the colorimetric data of each lock with a Minolta CM-3610d type spectrophotometer (Illuminant D65).

The locks of dyed hair are combed, moistened with a trickle of water at 35° C. and then passed between the fingers five times and squeezed dry between two fingers. A standard shampoo (Gamier Ultra Doux®) is then applied uniformly to the locks, in a proportion of 0.4 g of shampoo per gram of locks, the locks of hair being massaged gently along their length. Ten strokes are performed from the root to the end. The locks impregnated with shampoo are rinsed under a trickle of water at 35° C. while passing each lock between the fingers (15 passes) and then by squeezing them dry between two fingers before the next shampoo wash.

Once the desired number of shampoo washes has been performed, the locks of hair are combed and then dried with a hairdryer.

### 3. Colorimetric Evaluation

These evaluations were performed by spectrocolorimetry by measuring the colorimetric data L*, a* and b* using a Minolta CM-3610d spectrophotometer (Illuminant D65).

In this L* a* b* system, L* represents the lightness, a* represents the green/red color axis and b* represents the blue/yellow color axis. The higher the value of L, the lighter or less intense the color. Conversely, the lower the value of L, the darker or more intense the color. The higher the value of a*, the redder the shade, and the higher the value of b*, the yellower the shade.

By means of this system, it is assessed, on the one hand, whether the buildup of the hair color obtained with a composition according to the invention is acceptable and, on the other hand, whether the subsequent dyeing of the hair, i.e. before shampoo washing, is affected by successive shampoo washes. Thus, the color buildup, or ability of the fibers to be dyed, is evaluated by the variation in coloring between the locks of the dyed keratin fibers and the same keratin fibers before dyeing.

The color buildup on hair (ΔE), or the ability of the fibers to be dyed, is evaluated by the variation in coloring between locks of the dyed keratin fibers and the same fibers before dyeing according to the following equation:

$$Δ E = \sqrt{{( {L}^{*} - {L}_{0}^{*} )}^{2} + {( {a}^{*} - {a}_{0}^{*} )}^{2} + {( {b}^{*} - {b}_{0}^{*} )}^{2}}$$

In this equation, L*, a* and b* represent the values measured after dyeing the keratin fibers, and L₀*, a₀* and b₀* represent the values measured before dyeing the keratin fibers, which are natural gray (NG) hair containing at least 90% of white hair strands.

The higher the value of the color buildup ΔE, the more the fibers have been dyed and the more effective the dyeing.

The color persistence, or the resistance of the color of the keratin fibers to external attacking factors such as shampoo washing, is evaluated by the difference in buildup ΔE before and after attack, in this case successive shampoo washes, of said fibers. The smaller the variation, the more persistent the coloring.

### 4. Skin Application

### One-Gesture Protocol

In the case of a one-step application, a composition containing a polymer CP bearing acetoacetate functions according to the invention and a crosslinking agent R which may be either an amine compound or a thiol compound is prepared using a Speed Mixer (2 minutes at 3500 rpm). This composition is applied to a Bioskin type vitro support (elastomeric skin-simulating support) using a film spreader (wet thickness 100 μm). The deposit is left to dry for 24 hours.

### Two-Gesture Protocol

In the case of a two-step application, the composition, of the “base coat” type, containing a polymer bearing acetoacetate functions is prepared using a Speed Mixer (2 minutes at 3500 rpm). This “base coat” is applied to a Bioskin type vitro support (elastomeric skin-simulating support) using a film spreader (wet thickness 100 μm). The deposit is left to dry for 24 hours.

A composition, of the “top coat” type, containing an amine compound is then applied. After 24 hours of drying, the deposits obtained are evaluated according to points 5 to 7 below.

### 5. Resistance to Olive Oil or Water

0.5 mL of olive oil or water is applied to the deposit. After 5 minutes, the olive oil or water is removed by wiping 15 times with cotton wool. The deterioration of the deposit resulting from its contact with olive oil or water is thus observed.

The resistance is evaluated according to the following scale:
+++: no attack of the deposit, which is as it was originally,
++: a little transfer but the deposit is as it was originally,
+: the deposit is slightly altered and some transfer is observed.

### 6. Test of Adhesion to the Support

A piece of adhesive tape (Scotch® Magic™ 810 from the company 3M; w=19 mm, L=5 cm) is applied to the deposit. A mass of about 1070 g is placed on the piece of adhesive tape for 30 seconds. The adhesive tape is then removed and applied to an object slide to observe the amount of residual deposit on the adhesive tape. The adhesion of the film to the support is thus evaluated.

The adhesion of the deposit to the support is evaluated according to the following scale:
+++: no transfer of the deposit, which is as it was originally,
++: a little transfer but the deposit is as it was originally,
+: transfer of the deposit.

### 7. Fragmentation Test

The Bioskin® plate with the deposit on it is manually stretched 10 times.

The degree of fragmentation (or cracking) of the deposit is then observed to determine whether or not it is cohesive.

The fragmentation of the film is evaluated according to the following scale:
+++: no fragmentation, the deposit is intact,
++: fragmentation is observed but no holes are formed in the deposit,
+: fragmentation is observed, accompanied by holes in the deposit.

### Example A: Preparation of Copolymer 1: Isobornyl Acrylate/Acetoacetoxyethyl Methacrylate (90/10)

135 g of isobornyl acrylate monomer, 30 g of acetoacetoxyethyl methacrylate, 100 g of an isododecane/ethyl acetate (70/30) solvent, and 100 g of isododecane alone are introduced into a 1 L pilot reactor.

The medium is degassed with argon and then heated to 90° C. with stirring (100 rpm).

Once the reaction medium reaches the temperature of 90° C., 338 g of a mixture composed of 200 g of isododecane, 135 g of isobornyl acrylate monomer and 3 g of Trigonox 21S initiator are poured in over one hour.

The reaction medium is kept for 7 hours at 90° C. The next day, the reaction medium is stripped with 2×200 mL of cold isododecane.

On conclusion of the reaction, a dry extract of 43% polymer in isododecane is obtained.

This polymer is characterized by gel permeation chromatography (GPC). The results are detailed in the table below.

TABLE 2

| Mw (g/mol) | Mn (g/mol) | Ip |
|---|---|---|
| 158 100 | 10 900 | 14.5 |

### Example B: Preparation of Copolymer 2: Isobornyl Acrylate/2-ethylhexyl Acrylate/Acetoacetoxyethyl Methacrylate (60/30/10)

90 g of isobornyl acrylate monomer, 45 g of 2-ethylhexyl acrylate, 30 g of acetoacetoxyethyl methacrylate, 100 g of an isododecane/ethyl acetate (70/30) solvent and 100 g of isododecane alone are introduced into a 1 L pilot reactor.

The medium is degassed with argon and then heated to 90° C. with stirring (100 rpm).

Once the reaction medium reaches the temperature of 90° C., 338 g of a mixture composed of 200 g of isododecane, 90 g of isobornyl acrylate, 45 g of 2-ethylhexyl acrylate and 3 g of Trigonox 21S initiator are poured in over one hour.

The reaction medium is kept for 7 hours at 90° C. The next day, the reaction medium is stripped with 2×200 mL of cold isododecane to remove the residual monomers.

On conclusion of the reaction, a dry extract of 43% polymer in isododecane is obtained.

This polymer is characterized by GPC. The results are detailed in the table below.

TABLE 3

| Mw (g/mol) | Mn (g/mol) | Ip |
|---|---|---|
| 138 800 | 12 400 | 11.2 |

### Example C: Preparation of Copolymer 3: Isobornyl Acrylate/Butyl Acrylate/Acetoacetoxyethyl Methacrylate (60/30/10)

90 g of isobornyl acrylate, 45 g of butyl acrylate, 30 g of acetoacetoxyethyl methacrylate, 100 g of an isododecane/ethyl acetate (70/30) solvent and 100 g of isododecane alone are introduced into a 1 L pilot reactor.

The medium is degassed with argon and then heated to 90° C. with stirring (100 rpm).

Once the reaction medium reaches the temperature of 90° C., 338 g of a mixture composed of 200 g of isododecane, 90 g of isobornyl acrylate, 45 g of butyl acrylate and 3 g of Trigonox 21S initiator are poured in over one hour.

The reaction medium is kept for 7 hours at 90° C. The next day, the reaction medium is stripped with 2×200 mL of cold isododecane to remove the residual monomers.

On conclusion of the reaction, a dry extract of 43% polymer in isododecane is obtained.

### Example D: Preparation of Copolymer 4: Isobornyl Acrylate/2-ethylhexyl Acrylate/PDMS Methacrylate/Acetoacetoxyethyl Methacrylate (60/25/5/10)

90 g of isobornyl acrylate, 37.5 g of 2-ethylhexyl acrylate, 7.5 g of PDMS methacrylate, 30 g of acetoacetoxyethyl methacrylate, 100 g of an isododecane/ethyl acetate (70/30) solvent and 100 g of isododecane alone are introduced into a 1 L pilot reactor.

The medium is degassed with argon and then heated to 90° C. with stirring (100 rpm).

Once the reaction medium reaches the temperature of 90° C., 338 g of a mixture consisting of 200 g of isododecane, 90 g of isobornyl acrylate, 37.5 g of 2-ethylhexyl acrylate, 7.5 g of PDMS methacrylate and 3 g of Trigonox 21 S initiator are poured in over one hour.

The reaction medium is kept for 7 hours at 90° C. The next day, the reaction medium is stripped with 2×200 mL of cold isododecane to remove the residual monomers.

On conclusion of the reaction, a dry extract of 43% polymer in isododecane is obtained.

### Example E: Preparation of Copolymer 5: Isobutyl Acrylate/Tert-Butyl Acrylate/Acetoacetoxyethyl Methacrylate (25/65/10)

62.5 g of isobutyl acrylate, 162.5 g of tert-butyl acrylate, 25 g of acetoacetoxyethyl methacrylate, 2.5 g of Trigonox T21S radical initiator and 360 g of an isododecane/ethyl acetate (50/50) solvent are introduced into a 1 L pilot reactor. The medium is degassed with argon and then heated to 90° C. with stirring. The reaction medium is kept for 7 hours at 90° C. Stripping with 300 ml of isododecane is then performed to remove the residual monomers. On conclusion of the reaction, a dry extract of 48% polymer in isododecane is obtained.

### Example F: Preparation of Copolymer 6: Isobutyl Acrylate/Isobutyl Methacrylate/Acetoacetoxyethyl Methacrylate (30/60/10)

75 g of isobutyl acrylate, 150 g of isobutyl methacrylate, 25 g of acetoacetoxyethyl methacrylate, 2.5 g of Trigonox T21S radical initiator and 360 g of an isododecane/ethyl acetate (50/50) solvent are introduced into a 1 L pilot reactor. The medium is degassed with argon and then heated to 90° C. with stirring. The reaction medium is kept for 7 hours at 90° C. Stripping with 300 ml of isododecane is then performed to remove the residual monomers. On conclusion of the reaction, a dry extract of 49% polymer in isododecane is obtained.

### Examples 1-2: Compositions According to the Invention for Application to the Hair in One Step

The compositions of Examples 1 and 2 according to the invention comprising copolymer 2, as prepared in Example B above, are prepared from the contents shown in the table below. The contents are expressed as weight percentages relative to the total weight of the composition.

TABLE 4

| Compounds | Example 1 | Example 2 |
|---|---|---|
| Copolymer 2 of Example B | 34.88* | 34.88* |
| Red iron oxide (CI: 77491) (Sunpuro Red | 3 | 3 |
| Iron Oxide C33-8001 from the company | | |
| Sun) | | |
| (3-Aminopropyl)triethoxysilane (APTES) | / | 5 |
| (from the company Sigma-Aldrich) | | |
| Isododecane | qs 100 | qs 100 |

*percentage of active material

Tests of color buildup and resistance of the hair coloring to shampoo washing are performed with the compositions of Examples 1 and 2, according to the one-step protocol detailed in points 2 and 3 above in the “Methods and measurements” section.

The table below details the number of shampoo washes and reports the measured colorimetric values.

TABLE 5

| | L | a | b | ΔE |
|---|---|---|---|---|
| Untreated lock | 61.26 | 0.73 | 15.5 | |
| Example 1 before shampoo washing | 35.93 | 25.37 | 18.97 | 35.51 |
| Example 1 after 10 shampoo washes | 36.51 | 23.18 | 16.68 | 33.44 |
| Example 2 before shampoo washing | 38.29 | 21.88 | 16.97 | 31.27 |
| Example 2 after three shampoo washes | 39.95 | 21.16 | 16.45 | 29.54 |

It is seen from the above table that the treatment of keratin fibers with the compositions of the invention of Examples 1 and 2 makes it possible to dye said fibers intensely and chromatically with a very significant color buildup.

In addition, the tested compositions according to the invention confer resistance to the coloring even after several successive shampoo washes for the compositions of Examples 1 and 2 since no significant variation in the colorimetric parameters was observed or measured.

It was also visually observed that the chromatic coloring (notably in the reds) is unchanged between the coloring obtained just after application of the compositions of Examples 1 and 2 according to the invention, and after several successive shampoo washes. This observation is confirmed by a small variation in the ΔE values.

In addition, it was also observed that the keratin fibers appear individualized after application of the compositions of Examples 1 and 2 according to the invention, with a respected volume, this being the case even after several shampoo washes. Advantageously, the feel is also very pleasant.

### Examples 3-8 and 3a: Compositions According to the Invention for Application to the Hair in Two Steps

The compositions of Examples 3 to 8 according to the invention comprising copolymers 1, 2 or 4, as prepared in Examples A, B and D above, respectively, are prepared from the contents shown in the table below. The contents are expressed as weight percentages relative to the total weight of the composition under consideration.

TABLE 6

| | | Example | Example | Example | Example | Example | Example |
|---|---|---|---|---|---|---|---|
| Formula | Compounds | 3 | 4 | 5 | 6 | 7 | 8 |
| “Base | Copolymer 1 of Example A | / | / | / | / | / | 34.88* |
| coat” C1 | Copolymer 2 of Example B | 34.88* | 34.88* | / | / | 34.88* | / |
| | Copolymer 4 of Example D | / | / | 34.88* | 34.88* | / | / |
| | Red iron oxide (CI: 77491) (Sunpuro Red Iron | 3 | / | 3 | / | 3 | 3 |
| | Oxide C33-8001 from the company Sun) | | | | | | |
| | Yellow iron oxide (CI: 77492) (Sunpuro Yellow | / | 3 | / | / | / | / |
| | Iron Oxide C33-9001 from the company Sun) | | | | | | |
| | Nacre (mica coated with brown iron oxide (CI: | / | / | / | 3 | / | / |
| | 77491)) (Pearlescent Pigment Prestige Soft Beige | | | | | | |
| | from the company Sudarshan Chemical) | | | | | | |
| | 2-Octyl-1-dodecanol | / | / | / | / | / | 12 |
| | Isododecane | qs 100 | qs 100 | qs 100 | qs 100 | qs 100 | qs 100 |
| “Top | Plant chitosan: poly(D-glucosamine) (Kionutrime | 5* | 5* | 5* | 5* | / | 5* |
| coat” CR | CSG* from the company Kytozyme) | | | | | | |
| | [4-6% (mercaptopropyl)methyl-siloxane]- | / | / | / | / | 5* | / |
| | dimethylsiloxane copolymer (SMS-042 from the | | | | | | |
| | company Gelest) | | | | | | |
| | Water | qs 100 | qs 100 | qs 100 | qs 100 | / | qs 100 |
| | Isododecane | / | / | / | / | qs 100 | / |

*percentage of active material

The composition of Example 3a according to the invention comprising copolymer 5, as prepared in Example E above, is prepared from the contents shown in Table 6a below. The contents are expressed as weight percentages relative to the total weight of the composition under consideration.

TABLE 6a

| Formulations | Compounds | Example 3a |
|---|---|---|
| “Base coat” | Copolymer 5 of Example E | 15* |
| C1 | Red iron oxide (CI: 77491) Sunpuro Red | 6 |
| | Iron Oxide C33-8001 from the company | |
| | Sun | |
| | Ethanol | 39.5 |
| | Isododecane | qs 100 |
| “Top coat” | [4-6% (mercaptopropyl)methylsiloxane] - | 8 |
| CR | dimethylsiloxane copolymer (from the | |
| | company Gelest) | |
| | Isododecane | qs 100 |

*percentage of active material

Color buildup and shampoo washing resistance tests are performed for the “base coat” and “top coat” combinations of the compositions of Examples 3 to 8 according to the invention, according to the two-step protocol detailed in points 2 and 3 above in the “Methods and measurements” section.

Color buildup tests are performed for the “base coat” and “top coat” combinations of the composition of Example 3a according to the invention, according to the two-step protocol detailed in points 2 and 3 above in the “Methods and measurements” section.

The tables below detail the number of shampoo washes and report the measured colorimetric values.

TABLE 7

| | L | a | b | ΔE |
|---|---|---|---|---|
| Untreated lock | 61.26 | 0.73 | 15.5 | |

Color buildup before and after 10 successive shampoo washes

| | | | | |
|---|---|---|---|---|
| Example 3 before shampoo washing | 45.58 | 20.44 | 18.37 | 26.71 |
| Example 3 after 10 shampoo washes | 45.58 | 20 | 18.52 | 26.4 |
| Example 7 before shampoo washing | 36.69 | 26.25 | 20.4 | 35.76 |
| Example 7 after 10 shampoo washes | 37.81 | 24.47 | 19.22 | 33.58 |

Color buildup

| | | | | |
|---|---|---|---|---|
| Example 4 before shampoo washing | 56.33 | 6.86 | 26.02 | 13.84 |
| Example 5 before shampoo washing | 38.14 | 21.07 | 16.86 | 30.83 |
| Example 6 before shampoo washing | 48.78 | 14.36 | 19.11 | 18.83 |
| Example 8 before shampoo washing | 35.54 | 23.99 | 18.1 | 34.78 |

TABLE 7a

| | L | a | b | ΔE |
|---|---|---|---|---|
| Untreated lock | 55.69 | 1.58 | 12.64 | |

Colour buildup

| | | | | |
|---|---|---|---|---|
| Example 3a before | 38.13 | 15.33 | 12.38 | 24.45 |
| shampoo washing | | | | |

It is seen from the above tables that the treatment of keratin fibers with the compositions of the invention of Examples 3 to 8 and 3a makes it possible to dye said fibers intensely and chromatically with a very significant color buildup.

In addition, the tested compositions according to the invention confer resistance to the coloring even after several successive shampoo washes for the compositions of Examples 3 and 7 since no significant variation in the colorimetric parameters was observed or measured. It was also visually observed that the chromatic coloring (notably in the yellows and reds) is unchanged between the coloring obtained just after application of the compositions of Examples 3 and 7 according to the invention, and after ten successive shampoo washes. This observation is confirmed by a small variation in the ΔE value.

In addition, it was also observed that the keratin fibers appear individualized after application of the compositions of Examples 3 to 8 according to the invention, with a respected volume, this being the case even after several shampoo washes. Advantageously, the feel is also very pleasant.

### Examples 9-10: Compositions According to the Invention for Application to the Hair in Three Steps

The compositions of Examples 9 and 10 according to the invention comprising copolymer 2, as prepared in Example B above, are prepared from the contents shown in the table below. The contents are expressed as weight percentages relative to the total weight of the composition under consideration.

TABLE 8

| | | Example | Example |
|---|---|---|---|
| Formula | Compounds | 9 | 10 |
| “Base coat” | Copolymer 2 of Example B | 34.88* | 34.88* |
| C1 | Isododecane | qs 100 | qs 100 |
| “Top coat” | Plant chitosan: poly(D- | 5* | 5* |
| CR | glucosamine) (Kionutrime | | |
| | CSG* from the company | | |
| | Kytozyme) | | |
| | Water | qs 100 | qs 100 |
| “Pigment” | Yellow iron oxide (CI: 77492) | 6* | / |
| CAC | (Sunpuro Yellow Iron Oxide | | |
| | C33-9001 from the company Sun) | | |
| | Water | qs 100 | qs 100 |

*percentage of active material

Color buildup and shampoo washing resistance tests are performed for the “base coat”, “top coat” and “pigment” combination of Examples 9 and 10 according to the invention, according to the three-step protocol detailed in points 2 and 3 above in the “Methods and measurements” section.

The table below details the number of shampoo washes and reports the measured colorimetric values.

TABLE 9

| | L | a | b | ΔE |
|---|---|---|---|---|
| Untreated lock | 61.26 | 0.73 | 15.5 | |
| Example 9 before shampoo washing | 53.38 | 13.43 | 41.54 | 30.03 |
| Example 9 after three shampoo washes | 55.08 | 14.61 | 43.67 | 32.01 |
| Example 10 before shampoo washing | 51.35 | 12.99 | 39.37 | 28.61 |
| Example 10 after three shampoo washes | 52.72 | 12.74 | 37.58 | 26.54 |

It is seen from the above table that the treatment of keratin fibers with the compositions of the invention of Examples 9 and 10 makes it possible to dye said fibers intensely and chromatically with a very significant color buildup.

In addition, the tested compositions according to the invention confer resistance to the coloring even after three successive shampoo washes for the compositions of Examples 9 and 10 since no significant variation in the colorimetric parameters was observed or measured.

It was also visually observed that the chromatic coloring (notably in the yellows) is unchanged between the coloring obtained just after application of the composition of Examples 9 and 10 according to the invention, and after three successive shampoo washes. This observation is confirmed by a small variation in the ΔE value.

In addition, it was also observed that the keratin fibers appear individualized after application of the compositions of Examples 9 and 10 according to the invention, with a respected volume, this being the case even after several shampoo washes. Advantageously, the feel is also very pleasant.

### Examples 11-18: Compositions According to the Invention for Application to the Skin

The compositions of Examples 111 18 according to the invention comprising copolymers 1, 2, 3 or 4, as prepared in Examples A, B, C and D above, respectively, are prepared from the contents shown in the table below. The contents are expressed as weight percentages relative to the total weight of the composition under consideration.

TABLE 10

| Formula | Compounds | Ex. 11 | Ex. 12 | Ex. 13 | Ex. 14 | Ex. 15 | Ex. 16 | Ex. 17 | Ex. 18 |
|---|---|---|---|---|---|---|---|---|---|
| “Base coat” | Copolymer 1 of Example A | 58.14* | 58.14* | / | / | / | / | 58.14* | 58.14* |
| C1 | Copolymer 2 of Example B | / | / | 58.14* | 58.14* | / | / | / | / |
| | Copolymer 3 of Example C | / | / | / | / | 58.14* | / | / | / |
| | Copolymer 4 of Example D | / | / | / | / | / | 58.14* | / | / |
| | Red iron oxide (CI: 77491) | / | 5 | / | / | / | / | / | / |
| | (Sunpuro Red Iron Oxide C33- | | | | | | | | |
| | 8001 from the company Sun) | | | | | | | | |
| | DC Red 7 (40% in isododecane) | 5 | / | 5 | 5 | 5 | 5 | 5 | 5 |
| | 2-Octyl-1-dodecanol | / | / | / | / | / | / | 20 | / |
| | Isononyl isononanoate | / | / | / | / | / | / | / | 20 |
| | Isododecane | qs 100 | qs 100 | qs 100 | qs 100 | qs 100 | qs 100 | qs 100 | qs 100 |
| “Top coat” | Poly(dimethylsiloxane) bis(3- | 5* | / | / | 5* | / | / | / | 5* |
| C2 | aminopropyl) terminated PDMS- | | | | | | | | |
| | diNH₂) (50 000 g/mol) (DMS- | | | | | | | | |
| | A35 from the company Gelest) | | | | | | | | |
| | Bis-cetearyl amodimethicone | / | 5* | / | / | 5* | 5* | 5* | / |
| | (from the company Momentive | | | | | | | | |
| | Performance Materials) | | | | | | | | |
| | (3-Aminopropyl)triethoxysilane | / | / | 5* | / | / | / | / | / |
| | (APTES) (from the company | | | | | | | | |
| | Sigma-Aldrich) | | | | | | | | |
| | Isododecane | qs 100 | qs 100 | qs 100 | qs 100 | qs 100 | qs 100 | qs 100 | qs 100 |

*percentage of active material

For each of the Examples 11 to 18 according to the invention, the evaluations are performed according to points 4 to 7 of the “Methods and measurements” section above. These evaluations are performed on successive deposits of “base coat” and then “top coat” compositions and reported in the table below.

TABLE 11

| | Water | Oil | Adhesion to | |
|---|---|---|---|---|
| Examples | resistance | resistance | the support | Fragmentation |
| Example 11 | +++ | +++ | +++ | + |
| Example 12 | +++ | +++ | +++ | +++ |
| Example 13 | +++ | ++ | +++ | +++ |
| Example 14 | +++ | ++ | +++ | +++ |
| Example 15 | +++ | ++ | +++ | +++ |
| Example 16 | +++ | ++ | +++ | + |
| Example 17 | +++ | +++ | ++ | +++ |
| Example 18 | +++ | +++ | ++ | +++ |

### Water Resistance

The deposits made with the “base coat” type compositions and then the “top coat” type compositions of Examples 11 to 18 according to the invention advantageously have very good water resistance.

Deposits that are resistant to daily chemical attack are thus advantageously obtained with the compositions according to the invention.

### Oil Resistance

The deposits made with the “base coat” type composition and then the “top coat” type composition of Examples 11 to 18 according to the invention also advantageously have very good oil resistance.

Deposits that are resistant to daily chemical attack are thus advantageously obtained with the compositions according to the invention.

### Adhesion to the Support

The successive application of the “top coat” type composition onto the “base coat” type composition, according to Examples 11 to 18, makes it possible to significantly improve the adhesion notably to the support of the final cosmetic product. The compositions of Examples 17 and 18 have good adhesion to the support and the compositions of Examples 11 to 16 have very good adhesion to the support.

### Fragmentation

The successive application of the “top coat” type composition onto the “base coat” type composition, according to Examples 11 to 18, significantly improves the cohesion of the deposits obtained.

The compositions of Examples 12 to 15, 16 and 17 show very good cohesion.

### Examples 19, 19a and 19b: Compositions According to the Invention for Hairstyling Application

The composition of Example 19 according to the invention comprising copolymer 2, as prepared in Example B above, is prepared from the contents indicated in the table below. The contents are expressed as weight percentages relative to the total weight of the composition under consideration.

TABLE 12

| Compounds | Example 19 |
|---|---|
| Copolymer 2 of Example B | 10* |
| Isododecane | qs 100 |

*percentage of active material

For Example 19 according to the invention, an evaluation is made according to point 1 of the “Methods and measurements” section above. A test was performed according to the styling application protocol in point 1 relative to a comparative example consisting in treating a lock only with isododecane.

The lock lengths measured after application and 24 hours after application are reported in the table below:

TABLE 13

| | Measurement | Measurement |
|---|---|---|
| | after | 24 h after |
| Treatment applied | application | application |
| to the locks | (cm) | (cm) |
| Isododecane (comparative) | 20.5 | 12 |
| Example 19 | 20.5 | 7 |

The composition of Example 19 allows corporization of the hair fiber. The curls obtained after treatment of the keratin fibers with this composition are very pronounced, with curl heights, i.e. lock lengths after treatment, which are reduced by 30% to 70%, whereas the curls obtained with isododecane are not very pronounced, with a very small curl radius. The composition of Example 19 according to the invention makes it possible to keep a very pronounced curl effect even after 24 hours, unlike the lock treated with isododecane. Significant retention of the curl effect was also observed for locks treated with the composition of Example 19, without noting after 24 hours any difference in the height of the curled lock.

The compositions of Examples 19a and 19b according to the invention comprising, respectively, copolymer 5 as prepared in Example E above or copolymer 6 as prepared in Example F above are prepared from the contents indicated in the table below. The contents are expressed as weight percentages relative to the total weight of the composition under consideration.

TABLE 12a

| Compounds | Example 19a | Example 19b |
|---|---|---|
| Copolymer 5 of Example E | 10* | 0 |
| Copolymer 6 of Example F* | 0 | 10* |
| 50/50 isododecane/ethanol | 90 | 90 |

*percentage of active material

For Examples 19a and 19b according to the invention, an evaluation is made according to point 1 of the “Methods and measurements” section above. A test was performed according to the styling application protocol in point 1 relative to a comparative example consisting in treating a lock only with a 50/50 isododecane/ethanol mixture.

The lock lengths measured after application and 24 hours after application are reported in the table below:

TABLE 13a

| | Measurement | Measurement |
|---|---|---|
| | after | 24 h after |
| | application | application |
| Locks | (cm) | (cm) |
| 50/50 isododecane/ethanol | 20.5 | 15 |
| (comparative) | | |
| Example 19a | 20.5 | 10 |
| Example 19b | 20.5 | 9 |

The compositions of Examples 19a and 19b allow corporization of the hair fiber. The curls obtained after treatment of the keratin fibers with these compositions are very pronounced, with curl heights, i.e. lock lengths after treatment, which are reduced by 30% to 70%, whereas the curls obtained with the isododecane/ethanol mixture are not very pronounced, with a very small curl radius. The compositions of Examples 19a and 19b according to the invention make it possible to keep a very pronounced curl effect even after 24 hours, unlike the lock treated with the 50/50 isododecane/ethanol mixture. Significant retention of the curl effect was observed, without noting after 24 hours any difference in the height of the curled lock.

### Examples 20-24, 20a and 20b: Compositions According to the Invention for One-Step Application to the Skin

The compositions of Examples 20 to 24 according to the invention comprising copolymer 2, as prepared in Example B above, are prepared from the contents indicated in Table 14 below. The contents are expressed as weight percentages relative to the total weight of the composition.

The compositions of Examples 20a and 20b according to the invention comprising copolymer 5, as prepared in Example E above, are prepared from the contents shown in Table 14a below. The contents are expressed as weight percentages relative to the total weight of the composition.

For Examples 23 and 24, the crosslinking is performed in the presence of base (DBU) according to the conventional methods known to those skilled in the art (see for example Progress in Organic Coatings, X. He et al., 129, 21-25, (2019) https://doi.org/10.1016/j.porgcoat.2018.12.015, ibid Dongdong Xu et al., 135, 510-516 (2019), https://doi.org/10.1016/j.porgcoat.2019.06.026).

TABLE 14

| | Example | Example | Example | Example | Example |
|---|---|---|---|---|---|
| Compounds | 20 | 21 | 22 | 23 | 24 |
| Copolymer 2 of Example B | 58.14* | 58.14* | 58.14* | 58.14* | 58.14* |
| Red iron oxide (CI: 77491) (Sunpuro Red Iron Oxide C33-8001 | 5 | 5 | 5 | 5 | 5 |
| from the company Sun) | | | | | |
| Titanium butoxide (CAS No.: 5593-70-4) (from the company | 0.5* | / | / | / | / |
| Sigma-Aldrich) | | | | | |
| Aluminum lactate (from the company Sigma-Aldrich) | / | 5* | / | / | / |
| Basic aluminum acetate (from the company Sigma-Aldrich) | / | / | 5* | / | / |
| DBU 1,8-diazabicyclo[5.4.0]undec-7-ene (from the company | / | / | / | 1 | 1 |
| Sigma-Aldrich) | | | | | |
| Terephthalaldehyde (from the company Sigma-Aldrich) | / | / | / | 2.5 | / |
| Trimethylolpropane triacrylate (from the company Sigma- | / | / | / | / | 5 |
| Aldrich) | | | | | |
| Isododecane | qs 100 | qs 100 | qs 100 | qs 100 | qs 100 |

*percentage of active material

TABLE 14a

| Compounds | Example 20a | Example 20b |
|---|---|---|
| Copolymer 5 of Example E | 20 | 20 |
| Bis-cetearyl amodimethicone | / | 5 |
| from the company Momentive | | |
| Performance Materials) | | |
| DC Red 7 | 6 | 6 |
| 50/50 isododecane/ethanol | qs 100 | qs 100 |

*percentage of active material

For each of the Examples 20 to 24, 20a and 20b according to the invention, the evaluations are made according to points 4 to 7 of the “Methods and measurements” section above. These evaluations are performed on deposits of the compositions according to the invention and reported in the tables below.

TABLE 15

| | Water | Oil | Adhesion to | |
|---|---|---|---|---|
| Examples | resistance | resistance | the support | Fragmentation |
| Example 20 | +++ | + | ++ | ++ |
| Example 21 | ++ | + | +++ | +++ |
| Example 22 | +++ | + | +++ | +++ |
| Example 23 | +++ | + | ++ | +++ |
| Example 24 | +++ | + | ++ | +++ |

TABLE 15a

| | Adhesion to | | Oil | Water |
|---|---|---|---|---|
| Properties | the support | Fragmentation | resistance | resistance |
| Example 20a | + | + | ++ | +++ |
| Example 20b | +++ | + | ++ | +++ |

### Water Resistance

The deposits made with the compositions of Examples 20 to 24, 20a and 20b according to the invention advantageously have very good water resistance.

Deposits that are resistant to daily chemical attack are thus advantageously obtained with the compositions according to the invention.

### Oil Resistance

The deposits made with the compositions of Examples 20 to 24 according to the invention also advantageously have good oil resistance. The deposits made with the compositions of Examples 20a and 20b according to the invention advantageously also have good oil resistance.

Deposits that are resistant to daily chemical attack are thus advantageously obtained with the compositions according to the invention and notably with compositions 20a and 20b.

### Adhesion to the Support

The compositions of Examples 20, 23 and 24 according to the invention show good adhesion to the support and the compositions of Examples 20b, 21 and 22 according to the invention show very good adhesion to the support.

### Fragmentation

The composition of Example 20 according to the invention shows good cohesion, composition 20b according to the invention shows moderate cohesion and the compositions of Examples 21 to 24 according to the invention show very good cohesion.

### Example 25: Compositions According to the Invention for Application to the Hair in Two Steps

The compositions of Example 25 according to the invention comprising copolymer 2, as prepared in Example B above, are prepared from the contents indicated in the table below. The contents are expressed as weight percentages relative to the total weight of the composition under consideration.

TABLE 16

| Formula | Compounds | Example 25 |
|---|---|---|
| “Base coat” C1 | Copolymer of Example B | 15* |
| | Red iron oxide (CI: 77491) (Sunpuro | 6 |
| | Red Iron Oxide C33-8001 from the | |
| | company Sun) | |
| | Water | qs 100 |
| “Top coat” CR | Titanium butoxide (CAS No.: 5593-70-4) | 5* |
| | (from the company Sigma-Aldrich) | |
| | Ethanol | qs 100 |

*percentage of active material

After dyeing, the color buildup is satisfactory. The color obtained after treatment of the keratin fibers is esthetic, intense and chromatic.

It was also visually observed that the chromatic coloring (notably in the reds) is unchanged between the coloring obtained just after application of the compositions of Example 25 according to the invention, and after five successive shampoo washes.

In addition, it was also observed that the keratin fibers appear individualized after application of the compositions of Example 25 according to the invention, with a respected volume, this being the case even after several shampoo washes. Advantageously, the feel is also very pleasant.

### Example 26: Compositions According to the Invention for Application to the Skin in Two Steps

The compositions of Example 26 according to the invention comprising copolymer 5, as prepared in Example E above, are prepared from the contents indicated in the table below.

The contents are expressed as weight percentages relative to the total weight of the composition under consideration.

TABLE 17

| Formulations | Compounds | Example 26 |
|---|---|---|
| “Base coat” C1 | Copolymer 5 of Example E | 20* |
| | DC Red 7 | 6 |
| | (from the company Sensient) | |
| | 50/50 isododecane/ethanol | qs 100 |
| “Top coat” CR | bis-cetearyl amodimethicone | 5 |
| | (from the company Momentive | |
| | Performance Materials) | |
| | Isododecane | qs 100 |

*percentage of active material

For Example 26 according to the invention, the evaluations are made according to points 4 to 7 of the “Methods and measurements” section above. These evaluations are performed on the deposit of composition 26 according to the invention and reported in the tables below.

TABLE 18

| | Adhesion to | | Oil | Water |
|---|---|---|---|---|
| Properties | the support | Fragmentation | resistance | resistance |
| Base coat alone | + | + | + | +++ |
| Example 26 | +++ | ++ | ++ | +++ |

### Water Resistance

The deposits made with the composition of Example 26 according to the invention advantageously have very good water resistance.

Deposits that are resistant to daily chemical attack are thus advantageously obtained with the compositions according to the invention.

### Oil Resistance

The deposits made with the composition of Example 26 according to the invention also advantageously have good oil resistance.

Deposits that are resistant to daily chemical attack are thus advantageously obtained with the compositions according to the invention.

### Adhesion to the Support

The deposits made with the composition of Example 26 according to the invention show very good adhesion to the support.

### Fragmentation

The deposits made with the composition of Example 26 according to the invention show good cohesion.

1. A composition, termed “C1”, comprising:
i) at least one fatty phase; and
ii) at least one copolymer, termed “CP”, obtained by polymerization of:
0% to 99% by weight, relative to the total weight of the monomers, of at least one monomer (A) chosen from 2-ethylhexyl acrylate, 2-ethylhexyl methacrylate, isobornyl acrylate, isobornyl methacrylate, and mixtures thereof;
1% to 20% by weight, relative to the total weight of the monomers, at least one monomer (B) of formula (I): 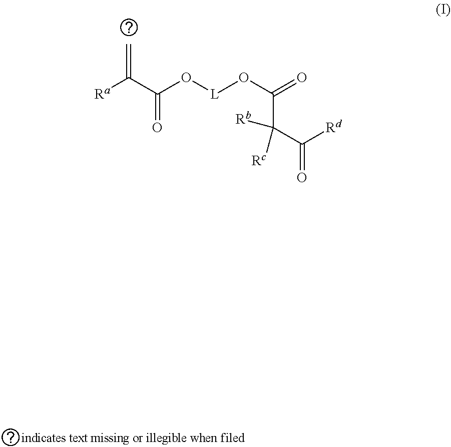
in which:
R^{a} represents a hydrogen atom or a linear or branched (C₁-C₄)alkyl group;
R^{b} and R^{c}, which may be identical or different, represent a hydrogen atom or a linear or branched (C₁-C₄)alkyl group;
R^{d} represents a linear or branched (C₁-C₄)alkyl group;
L represents a linear or branched (C₁-C₆)alkylene or cycloalkylene group; and
0% to 99% by weight of at least one monomer (C) chosen from C₁-C₄ alkyl acrylates, C₁-C₄ alkyl methacrylates, silicone macromonomers and mixtures thereof, relative to the total weight of the monomers,
it being understood that the copolymer termed “CP” is obtained by polymerization of at least one monomer (B) with at least one monomer (A) or at least one monomer (C).

2. (canceled)

3. The composition as claimed in claim 1, in which said copolymer termed “CP” is free of monomer (A).

4. The composition as claimed in claim 1, in which said copolymer CP comprises from 5% to 99% by weight, relative to the total weight of the monomers, of at least one monomer (A) chosen from 2-ethylhexyl acrylate, 2-ethylhexyl methacrylate, isobornyl acrylate, isobornyl methacrylate, and mixtures thereof and from 0% to 45% by weight of at least one monomer (C) chosen from C1-C4 alkyl acrylates, C1-C4 alkyl methacrylates, silicone macromonomers, and mixtures thereof, relative to the total weight of the monomers.

5. The composition as claimed in claim 1, in which said copolymer CP comprises from 50% to 99% by weight, of at least one monomer (A) chosen from 2-ethylhexyl acrylate, 2-ethylhexyl methacrylate, isobornyl acrylate, isobornyl methacrylate, and mixtures thereof, relative to the total weight of the monomers.

6. The composition as claimed in claim 1, in which said copolymer CP comprises at least one monomer (A) chosen from 2-ethylhexyl acrylate, 2-ethylhexyl methacrylate, isobornyl acrylate, isobornyl methacrylate, and mixtures thereof, in an amount of less than 50% relative to the total weight of the monomers, relative to the total weight of the monomers.

7. (canceled)

8. The composition as claimed in claim 1, in which said copolymer CP is free of monomer (C).

9. The composition as claimed in claim 1, in which said copolymer CP comprises as monomer (C) at least one monomer chosen from C₁-C₄ alkyl acrylates and/or C₁-C₄ alkyl methacrylates.

10. The composition as claimed in claim 1, in which said copolymer CP comprises as monomer (C) at least one silicone macromonomer.

11. The composition as claimed in claim 1, in which the copolymer CP comprises from 0% to 45% by weight of at least one monomer (C) chosen from C₁-C₄ alkyl acrylates, C₁-C₄ alkyl methacrylates, silicone macromonomers, and mixtures thereof, relative to the total weight of the monomers.

12. The composition as claimed in claim 1, in which the copolymer CP comprises from 80% to 99% by weight of at least one monomer (C) chosen from C₁-C₄ alkyl acrylates and C₁-C₄ alkyl methacrylates, and is free of monomer (A).

13. The composition as claimed in claim 1, in which the copolymer CP comprises from 80% to 99% by weight of at least one monomer (C) chosen from silicone macromonomers, and mixtures thereof with C₁-C₄ alkyl acrylates or with C₁-C₄ alkyl methacrylates, relative to the total weight of the monomers and is free of monomer (A).

14. The composition as claimed in claim 1, in which the copolymer CP comprises more than 30% by weight of at least one monomer (C) chosen from silicone macromonomers and mixtures thereof with C₁-C₄ alkyl acrylates or with C₁-C₄ alkyl methacrylates, relative to the total weight of the monomers, and at least one monomer (A) in a non-zero amount and less than 50% relative to the total weight of the monomers.

15. The composition as claimed in claim 1, in which the copolymer CP comprises more than 45% and less than 49% by weight of at least one monomer (C) chosen from C₁-C₄ alkyl acrylates, C₁-C₄ alkyl methacrylates, silicone macromonomers, and mixtures thereof, and at least one monomer (A) in an amount of from 50% to 99% relative to the total weight of the monomers.

16. (canceled)

17. The composition as claimed in claim 1, in which the copolymer is obtained by copolymerization of:
isobornyl acrylate and acetoacetoxyethyl methacrylate, in an isobornyl acrylate/acetoacetoxyethyl methacrylate mass ratio of 90/10,
isobornyl acrylate, 2-ethylhexyl acrylate and acetoacetoxyethyl methacrylate, in an isobornyl acrylate/2-ethylhexyl acrylate/acetoacetoxyethyl methacrylate mass ratio of 60/30/10,
isobornyl acrylate, butyl acrylate and acetoacetoxyethyl methacrylate, in an isobornyl acrylate/butyl acrylate/acetoacetoxyethyl methacrylate mass ratio of 60/30/10,
isobornyl acrylate, 2-ethylhexyl acrylate, PDMS methacrylate and acetoacetoxyethyl methacrylate, in an isobornyl acrylate/2-ethylhexyl acrylate/PDMS methacrylate/acetoacetoxyethyl methacrylate mass ratio of 60/25/5/10,
isobutyl acrylate, tert-butyl acrylate and acetoacetoxyethyl methacrylate, in an isobutyl acrylate/tert-butyl acrylate/acetoacetoxyethyl methacrylate mass ratio of 25/65/10,
isobutyl acrylate, isobutyl methacrylate and acetoacetoxyethyl methacrylate, in an isobutyl acrylate/isobutyl methacrylate/acetoacetoxyethyl methacrylate mass ratio of 30/60/10,
isobutyl acrylate, 2-ethylhexyl acrylate and acetoacetoxyethyl methacrylate, in an isobutyl acrylate/2-ethylhexyl acrylate/acetoacetoxyethyl methacrylate mass ratio of 88/2/10,
tert-butyl acrylate, ethyl acrylate, isobornyl acrylate and acetoacetoxyethyl methacrylate, in a tert-butyl acrylate/ethyl acrylate/isobornyl acrylate/acetoacetoxyethyl methacrylate mass ratio of 33/20/39/8,
isobornyl acrylate, 2-ethylhexyl acrylate, PDMS methacrylate and acetoacetoxyethyl methacrylate, in an isobornyl acrylate/2-ethylhexyl acrylate/PDMS methacrylate/acetoacetoxyethyl methacrylate mass ratio of 25/20/45/10;
isobutyl acrylate, PDMS methacrylate and acetoacetoxyethyl methacrylate, in an isobutyl acrylate/PDMS methacrylate/acetoacetoxyethyl methacrylate mass ratio of 65/25/10,
isobornyl acrylate, 2-ethylhexyl acrylate, PDMS methacrylate and acetoacetoxyethyl methacrylate, in an isobornyl acrylate/2-ethylhexyl acrylate/PDMS methacrylate/acetoacetoxyethyl methacrylate mass ratio of 40/12/46/2, or
isobornyl acrylate, isobutyl acrylate and acetoacetoxyethyl methacrylate, in an isobornyl acrylate/isobutyl acrylate/acetoacetoxyethyl methacrylate mass ratio of 52/46/2.

18. The composition as claimed in claim 1, comprising from 1% to 70% by weight of said copolymer, relative to the total weight of the composition.

19. The composition as claimed in claim 1, comprising at least one hydrocarbon-based oil.

20. The composition as claimed in claim 1, comprising from 15% to 98% by weight of hydrocarbon-based oil(s) relative to the total weight of the composition.

21. The composition as claimed in claim 16, comprising at least one nonvolatile oil different from said hydrocarbon-based oil.

22. The composition as claimed in claim 1, comprising at least one volatile hydrocarbon-based oil and optionally one or more nonvolatile oils.

23. The composition as claimed in claim 1, containing at least:
a copolymer obtained by copolymerizing at least isobornyl acrylate, 2-ethylhexyl acrylate and acetoacetoxyethyl methacrylate monomers in isododecane,
a copolymer obtained by copolymerizing at least isobornyl acrylate and acetoacetoxyethyl methacrylate monomers in isododecane, at least one nonvolatile oil different from isododecane,
a copolymer obtained by copolymerizing at least isobornyl acrylate, 2-ethylhexyl acrylate, PDMS methacrylate and acetoacetoxyethyl methacrylate monomers in isododecane,
a copolymer obtained by copolymerizing at least isobornyl acrylate and acetoacetoxyethyl methacrylate monomers in isododecane,
a copolymer obtained by copolymerizing at least isobornyl acrylate, butyl acrylate and acetoacetoxyethyl methacrylate monomers in isododecane,
a copolymer obtained by copolymerizing at least isobornyl acrylate, 2-ethylhexyl acrylate and acetoacetoxyethyl methacrylate monomers in isododecane and at least one pigment,
a copolymer obtained by copolymerizing at least isobornyl acrylate and acetoacetoxyethyl methacrylate monomers in isododecane, at least one nonvolatile oil different from isododecane, and at least one pigment,
a copolymer obtained by copolymerizing at least isobornyl acrylate, 2-ethylhexyl acrylate, PDMS methacrylate and acetoacetoxyethyl methacrylate monomers in isododecane, and at least one pigment,
a copolymer obtained by copolymerizing at least isobornyl acrylate and acetoacetoxyethyl methacrylate monomers in isododecane, and at least one pigment, or
a copolymer obtained by copolymerizing at least isobornyl acrylate, butyl acrylate and acetoacetoxyethyl methacrylate monomers in isododecane and at least one pigment.

24. A composition termed “C2” as claimed in claim 1, comprising at least one crosslinking agent, termed “R”, and optionally at least one cosmetic active agent, termed “CAA”, chosen from a) coloring agents chosen from pigments, direct dyes, and mixtures thereof, b) active agents for caring for keratin materials, c) UV-screening agents, and d) mixtures thereof; or
a composition termed “C3” as claimed in claim 1, comprising at least one cosmetic active agent, termed “CAA”, chosen from a) coloring agents chosen from pigments, direct dyes, and mixtures thereof, b) active agents for caring for keratin materials, c) UV-screening agents, and d) mixtures thereof.

25. (canceled)

26. The composition as claimed in claim 21, in which said crosslinking agent is chosen from i) (poly)amine compounds, ii) (poly)thiolated and/or (poly)hydroxylated compounds, iii) (poly)carbonyl compounds v) metal salts chosen from vₐ) metal alkoxides, v_{b}) (poly)(hydroxy)(C₁-C₆)alkylcarboxylates of transition metals or of post-transition metals, and v_{c}) salts of metals belonging to the group of the rare-earth metals, and vi) mixtures thereof.

27. The composition as claimed in claim 1, characterized in that it comprises at least:
a copolymer obtained by copolymerizing at least isobornyl acrylate, 2-ethylhexyl acrylate and acetoacetoxyethyl methacrylate monomers in isododecane, a crosslinking agent chosen from 3-aminopropyltriethoxysilane (APTES), poly(D-glucosamine), [4-6% (mercaptopropyl)methylsiloxane]dimethylsiloxane copolymer and bis(3-aminopropyl)-terminated poly(dimethylsiloxane) (PDMS-diNH₂),
a copolymer obtained by copolymerizing at least isobornyl acrylate and acetoacetoxyethyl methacrylate monomers in isododecane, a crosslinking agent chosen from poly(D-glucosamine), bis(3-aminopropyl)-terminated poly(dimethylsiloxane) (PDMS-diNH₂) bis-cetearyl amodimethicone, optionally at least one nonvolatile oil different from isododecane,
a copolymer obtained by copolymerizing at least isobornyl acrylate, 2-ethylhexyl acrylate, PDMS methacrylate and acetoacetoxyethyl methacrylate monomers in isododecane, a crosslinking agent chosen from poly(D-glucosamine) and bis-cetearyl amodimethicone,
a copolymer obtained by copolymerizing at least isobornyl acrylate, butyl acrylate and acetoacetoxyethyl methacrylate monomers in isododecane, bis-cetearyl amodimethicone,
a copolymer obtained by copolymerizing at least isobornyl acrylate, 2-ethylhexyl acrylate and acetoacetoxyethyl methacrylate monomers in isododecane, a crosslinking agent chosen from 3-aminopropyltriethoxysilane (APTES), poly(D-glucosamine), [4-6% (mercaptopropyl)methylsiloxane]dimethylsiloxane copolymer and bis(3-aminopropyl)-terminated poly(dimethylsiloxane) (PDMS-diNH2) and at least one pigment,
a copolymer obtained by copolymerizing at least isobornyl acrylate and acetoacetoxyethyl methacrylate monomers in isododecane, a crosslinking agent chosen from poly(D-glucosamine), bis(3-aminopropyl)-terminated poly(dimethylsiloxane) (PDMS-diNH2) bis-cetearyl amodimethicone, optionally at least one nonvolatile oil different from isododecane, and at least one pigment,
a copolymer obtained by copolymerizing at least isobornyl acrylate, 2-ethylhexyl acrylate, PDMS methacrylate and acetoacetoxyethyl methacrylate monomers in isododecane, a crosslinking agent chosen from poly(D-glucosamine) and bis-cetearyl amodimethicone, and at least one pigment, or
a copolymer obtained by copolymerizing at least isobornyl acrylate, butyl acrylate and acetoacetoxyethyl methacrylate monomers in isododecane, bis-cetearyl amodimethicone and at least one pigment.

28. The composition as claimed in claim 1, which is in the form of:
either a colored product for the lips;
or a skincare product, which may be colored;
or a haircare product.

29. A process for keratin materials, which consists in applying to said keratin materials a composition C1 as claimed in claim 1, optionally at least one crosslinking agent R, and optionally at least one cosmetic active agent CAA, said CAA being chosen from a) coloring agents chosen from pigments, direct dyes, and mixtures thereof, b) active agents for caring for keratin materials, c) UV-screening agents, and d) mixtures thereof.

30. A process for keratin materials, comprising at least the steps consisting in:
(a) placing all or part of the surface of said keratin materials in contact with a composition C1 as claimed in claim 1, and optionally containing at least one cosmetic active agent CAA; and
(b) placing all or part of said surface in contact with a composition termed “CR” different from said composition C1 and comprising at least one crosslinking agent termed “R”;
with it being possible for steps (a) and (b) to be performed simultaneously or sequentially, and it being understood that said process may optionally use at least one cosmetic active agent, termed “CAA”, contained in composition C1 and/or in composition CR and/or optionally in a composition which is different from C1 and from CR, termed “CAC”, said CAA being chosen from a) coloring agents chosen from pigments, direct dyes and mixtures thereof, b) active agents for caring for keratin materials, c) UV-screening agents, and d) mixtures thereof.

31. A process for making up the skin comprising a step of applying to said skin at least one composition C1 or C2 or C3 as claimed in claim 1, and at least one dyestuff, with said process comprising at least the two successive steps consisting in sequentially applying to said skin i) at least one composition C1, and ii) a crosslinking agent R or a composition CR containing at least one crosslinking agent R, it being understood that said composition C1 and/or said composition CR contains at least one dyestuff, or said process comprising at least the three successive steps consisting in sequentially applying to the skin i) a composition CAC containing at least one dyestuff, ii) a composition C1, and iii) a crosslinking agent R or a composition CR containing at least one crosslinking agent R, with said composition C1 and/or said composition CR optionally containing at least one dyestuff.

32. (canceled)

33. (canceled)

34. A process for dyeing keratin fibers comprising a step of applying to said keratin fibers a composition C1, C2 or C3 as claimed in claim 1 and at least one coloring agent, with said process comprising at least the two successive steps consisting in sequentially applying to said fibers i) at least one composition C1 as claimed in claim 1, and ii) a crosslinking agent R or a composition CR containing at least one crosslinking agent R, it being understood that said composition C1 and/or said composition CR contains at least one dyestuff, or said process comprising at least the three successive steps consisting in sequentially applying i) a composition CAC containing at least one dyestuff, ii) a composition C1 and iii) a crosslinking agent R or a composition CR containing at least one crosslinking agent R, with said composition C1 and/or said composition CR containing, where appropriate, at least one dyestuff.

35. A kit for keratin materials comprising:
a first compartment containing at least one composition C1 as claimed in claim 1;
a second compartment separate from said first compartment and comprising at least one crosslinking agent R; and
where appropriate, a third compartment separate from the first and second compartments and containing at least one cosmetic active agent, termed “CAA”, which may be identical to or different from those optionally contained in the first and/or second compartments;
it being understood that the first and/or second compartments may also contain one or more cosmetic active agents CAA, which may be identical or different, and in that said active agent CAA is chosen from a) coloring agents chosen from pigments, direct dyes and mixtures thereof, b) active agents for caring for keratin materials, c) UV-screening agents and d) mixtures thereof.
